(19) United States
(12) Patent Application Publication
(10) Pub. No.: US 2024/0174631 A1
(43) Pub. Date: May 30, 2024

(54) COMPOUNDS, COMPOSITIONS, AND METHODS FOR MODULATING FGF ACTIVITY

(71) Applicant: Recovery Therapeutics, Inc., Needham, MA (US)

(72) Inventors: Seth P. FINKLESTEIN, Needham, MA (US); Gregory D. CUNY, Houston, TX (US); Renato SKERLJ, Newton, MA (US); Soumya RAY, Quincy, MA (US); Stephen Douglas BARRETT, Hartland, MI (US); Kirk Lang OLSON, Canton, MI (US); Fred Lawrence CISKE, Dexter, MI (US); Paige Elizabeth HEIPLE, Ann Arbor, MI (US); Melissa Christine HOLT, Westminster, CO (US); James Bernard KRAMER, Sylvania, OH (US)

(21) Appl. No.: 18/276,981

(22) PCT Filed: Feb. 11, 2022
(86) PCT No.: PCT/US2022/016186

§ 371 Date: Aug. 11, 2023

Related U.S. Application Data
(60) Provisional application No. 63/148,865, filed on Feb. 12, 2021.

(51) Int. Cl. C07D 401/04; A61P 9/10; C07D 401/14; C07D 471/04

(52) U.S. Cl. CPC C07D 401/04; A61P 9/10; C07D 401/14; C07D 471/04

(57) ABSTRACT

The invention features compounds which modulate FGF activity, e.g., by enhancing the binding between FGF-2 and its receptors, e.g., FGF-R1. Also featured is a pharmaceutical composition containing one or more of the compounds, a method of treating an injury or a disease e.g., stroke, congenital hypogonadotropic hypogonadism, and viral infection, and a method of increasing spermatogenesis using the pharmaceutical composition.

### STATEMENT REGARDING FEDERALLY SPONSORED RESEARCH

This invention was made with government support under grant number 2R44 NS095381-02 from the National Institutes of Health. The government has certain rights to the invention.

### BACKGROUND OF THE INVENTION

Stroke is a medical condition caused by a lack of blood supply or bleeding into the brain. Stroke is a leading cause of death in the U.S., and affects approximately 800,000 people per year. Survivors of stroke live an average of seven years after stroke, and approximately 40% of survivors have severe mobility issues. There is a lack of effective treatments for stroke and methods for improving the recovery of stroke survivors.

Several growth factors, such as Fibroblast Growth Factors or FGFs, appear to stimulate the process of stroke recovery. In particular, FGF-2, a member of the FGF polypeptide family, supports the survival and outgrowth of a wide variety of neurons in the brain. Previous experimental studies in animals have shown that endogenous FGF-2 and its receptors, e.g., FGF-R1, are up-regulated after stroke, and exogenously administered FGF-2 can enhance spontaneous recovery after stroke, perhaps through increasing neuronal sprouting and new synapse formation in intact brain tissue surrounding the stroke and on the other side of the brain (Kawamata et al., Proc Natl Acad Sci. 94:8179-84, 1997). An additional mechanism may be stimulation of progenitor cell proliferation, migration, and differentiation in brain (Wada et al., Stroke. 34:2722-2728, 2003). However, FGF-2 is a 155-amino acid polypeptide of approximately 18 kDa, which makes the polypeptide challenging to use as a therapy for stroke and other brain injuries and diseases.

There exists a need for novel compounds and therapies to increase FGF-2 signaling activity and to enhance the binding between FGF-2 and its receptors, e.g., FGF-R1. Such compounds and therapies are useful in methods for treatment of stroke and other brain injuries and diseases, such as traumatic brain injury (TBI).

### SUMMARY OF THE INVENTION

The invention provides compounds, pharmaceutical compositions, and methods for treating various diseases, injuries, and disorders, e.g., modulated by FGF activity, and effecting other desirable outcomes. In particular, compounds of the invention may be used in the treatment of stroke, e.g., acute stroke and/or stroke in a recovery phase; congenital hypogonadotropic hypogonadism (e.g., Kallmann Syndrome); cerebral hemorrhage; traumatic brain injury (TBI); spinal cord injury (SCI); peripheral vascular disease (PVD); wounds, i.e., for wound healing; bone or cartilage injury; hearing loss; depression; anxiety; post-traumatic stress disorder (PTSD); substance abuse; peripheral nerve injury; hematopoietic disorders; amyotrophic lateral sclerosis (ALS); Alzheimer's disease; Parkinson's disease; heart disease; non-arteritic ischemic optic neuropathy (NAION); retinal artery occlusion; bronchopulmonary dysplasia, muscular dystrophy, anosmia, aging, memory disturbance, or viral infection.

In a first aspect, the invention features a compound having the structure of formula (I):

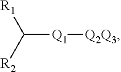

wherein R₁ is H, optionally substituted C₃-C₂₀ cycloalkyl, optionally substituted C₄-C₂₀ cycloalkenyl, optionally substituted C₁-C₁₅ heterocyclyl, or optionally substituted C₆-C₁₆ aryl; R₂ is optionally substituted C₃-C₂₀ cycloalkyl, optionally substituted C₄-C₂₀ cycloalkenyl, optionally substituted C₁-C₁₅ heterocyclyl, or optionally substituted C₆-C₁₆ aryl; Q₁ is optionally substituted 4-to-6 membered heterocyclylene containing at least one nitrogen atom; Q₂ is optionally substituted 5-to-7 membered heterocyclyl containing at least one nitrogen atom; and Q₃ is optionally substituted C₁-C₁₅ heterocyclyl, optionally substituted C₆-C₁₆ aryl, optionally substituted C₃-C₂₀ cycloalkyl, or optionally substituted C₄-C₂₀ cycloalkenyl, wherein Q₃ is fused to Q₂, or a pharmaceutically acceptable salt thereof.

In some embodiments, Q₁ is

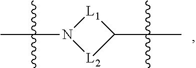

wherein each of L₁ and L₂ is, independently, optionally substituted C₁-C₂ alkylene. For example, Q₁ is

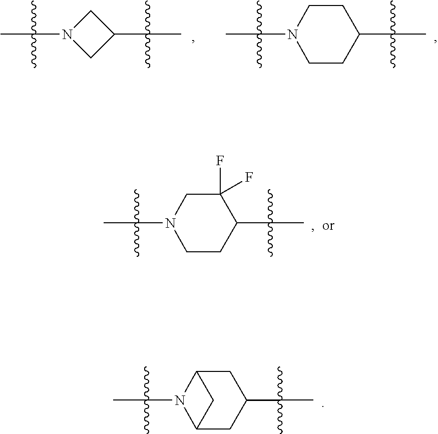 [SMILES: *C1CN(*)C1 ; *C1CCN(*)CC1 ; *C1CCN(*)CC1(F)F ; *C1CC2CC(C1)N2*]

In some embodiments, Q₂ is

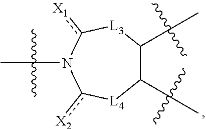

wherein each of L₃ and L₄ is independently absent or optionally substituted methylene; each [symbol] is independently a single or double bond; and each of X₁ and X₂ is independently O when [symbol] is a double bond or H when [symbol] is a single bond. For example, Q₂ is

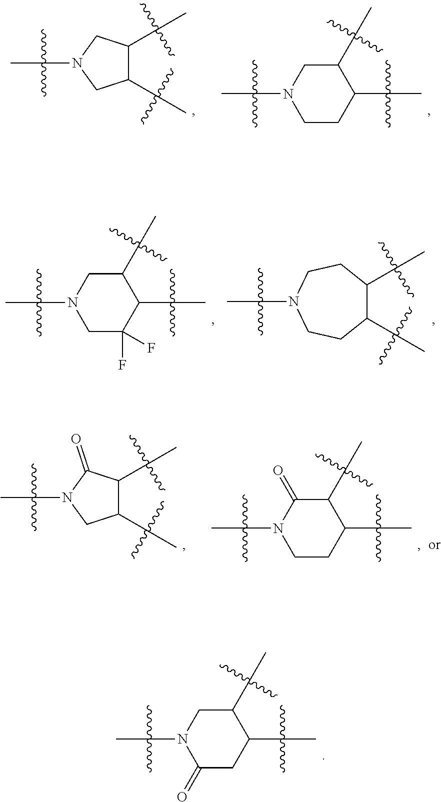

In some embodiments, Q₃ is optionally substituted phenyl or optionally substituted 6-membered aromatic heterocyclyl comprising at least one N atom.

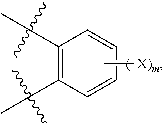

In some embodiments, Q₃ is, wherein m is 0-2, and each X is independently halo; CN; NO₂; optionally substituted C₁-C₆ alkyl; ORₐ, wherein Ra is H or optionally substituted C₁-C₆ alkyl; optionally substituted C₃-C₈ cycloalkyl; or NR_{b}R_{c}, wherein each of R_{b} and R_{c} is independently H or optionally substituted C₁-C₆ alkyl, or R_{b} and R_{c}, together with the N atom to which they are attached, form optionally substituted 3-7 membered heterocyclyl. For example, Q₃ is

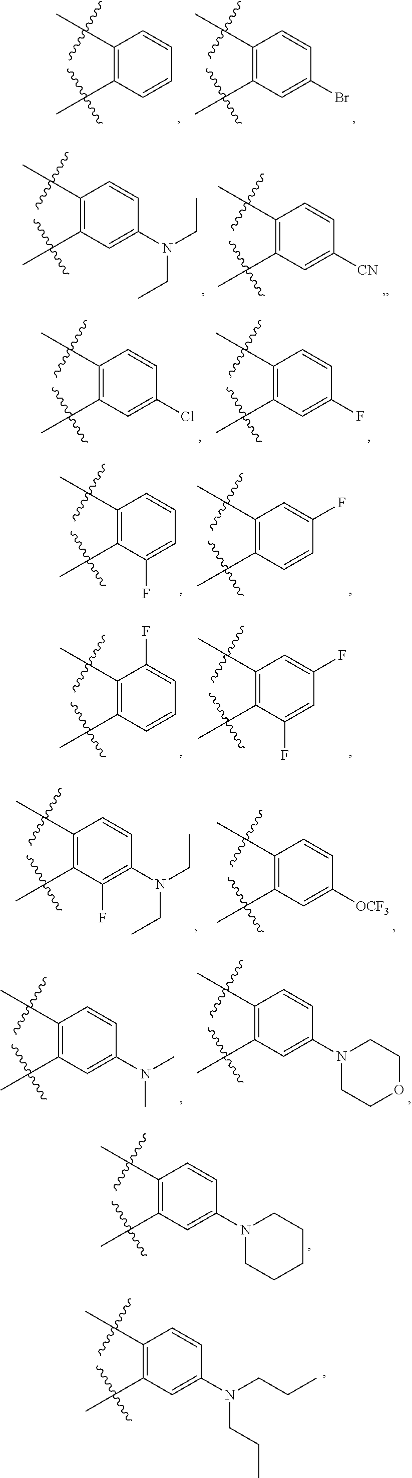

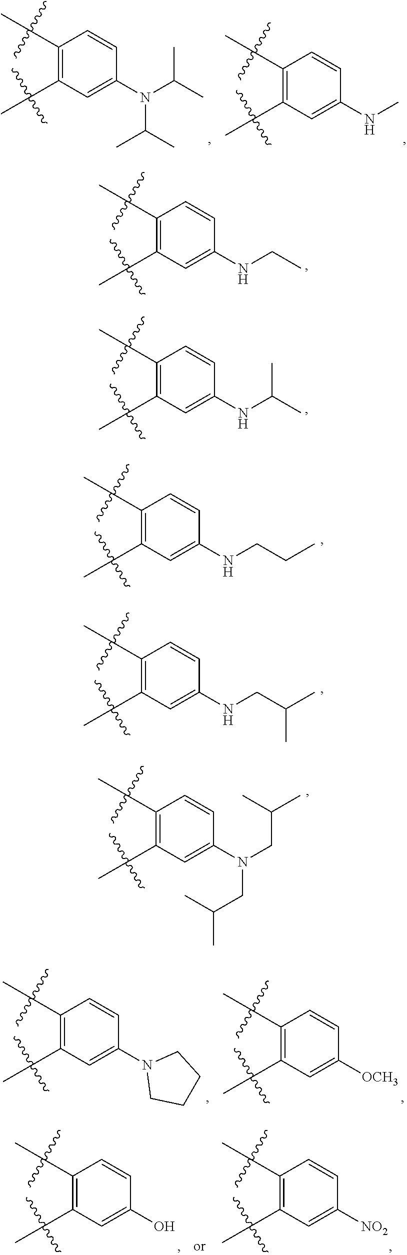

In some embodiments, Q₃ is

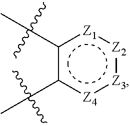

wherein each of Z₁, Z₂, Z₃, and Z₄ is N or CR_{c} and up to two (e.g., one or two) of Z₁, Z₂, Z₃, and Z₄ is N, wherein each R_{c} is independently H; halo, CN; NO₂; optionally substituted C₁-C₆ alkyl; ORₐ, wherein Ra is H or optionally substituted C₁-C₆ alkyl; optionally substituted C₃-C₈ cycloalkyl; or NR_{b}R_{c}, wherein each of R_{b} and R_{c} is independently H or optionally substituted C₁-C₆ alkyl, or R_{b} and R_{c}, together with the N atom to which they are attached, form optionally substituted 3-7 membered heterocyclyl. For example, Q₃ is

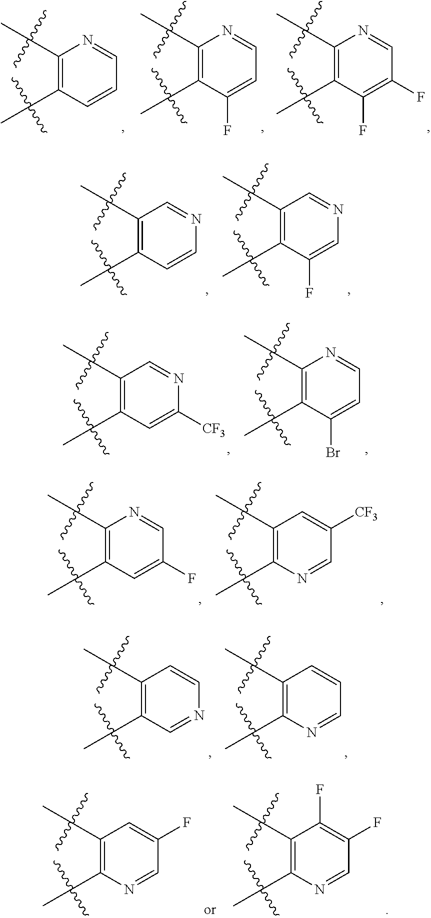

In some embodiments, the compound has the structure of formula (II):

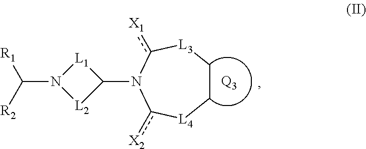

wherein L₁ and L₂ are each independently —C(X₃)₂— or —(C(X₃)₂)₂—, wherein each X₃ is independently H, halo, CN, NO₂, or C₁-C₆ alkyl; or an X₃ in L₁ and an X₃ in L₂ combine to form C₁-C₃ alkylene; each of L₃ and L₄ is independently absent or —C(X₄)₂—, wherein each X₄ is independently H, halo, CN, NO₂, or C₁-C₆ alkyl; each [symbol] is independently a single or double bond; and each of X₁ and X₂ is independently 0 when [symbol] is a double bond or H when [symbol] is a single bond.

In some embodiments, the compound has the structure of formula (IIA):

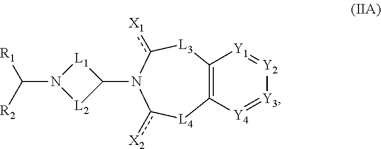

wherein each of Y₁, Y₂, Y₃, and Y₄ is independently N or CR₃, and at least one of Y₁, Y₂, Y₃, and Y₄ is CR₃, wherein each R₃ is independently H, halo, CN, NO₂, optionally substituted C₁-C₆ alkyl, optionally substituted C₂-C₆ alkenyl, optionally substituted C₂-C₆ alkynyl, optionally substituted C₃-C₈ cycloalkyl, optionally substituted C₄-C₈ cycloalkenyl, optionally substituted C₁-C₁₅ heterocyclyl, optionally substituted C₆-C₁₆ aryl, OR₄, SR₄, NR₄R₅, or C(O)NR₄R₅, wherein each of R₄ and R₅ is independently H, optionally substituted C₁-C₆ alkyl, optionally substituted C₂-C₆ alkenyl, optionally substituted C₂-C₆ alkynyl, optionally substituted C₃-C₈ cycloalkyl, optionally substituted C₄-C₈ cycloalkenyl, optionally substituted C₆-C₁₆ aryl, or optionally substituted C₁-C₁₅ heterocyclyl.

In some embodiments, each of Y₁, Y₂, Y₃, and Y₄ is CR₃. In certain preferred embodiments, each of Y₁, Y₂, and Y₃ is CH and Y₄ is CR₃. In other preferred embodiments, each of Y₁, Y₂, and Y₄ is CH and Y₃ is CR₃.

In some embodiments, R₃ is NR₄R₅, e.g., in which each of R₄ and R₅ is H or optionally substituted C₁-C₆ alkyl. For example, R₃ is

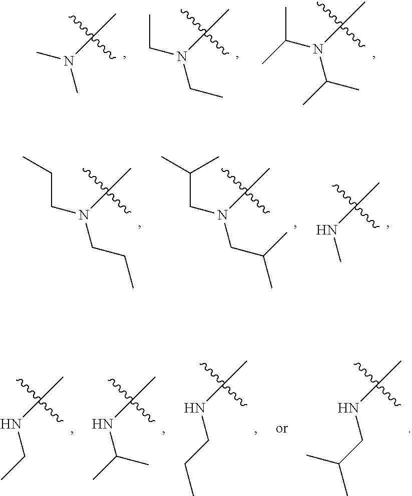

In some embodiments, R₃ is optionally substituted C₁-C₁₅ heterocyclyl. For example, R₃ is

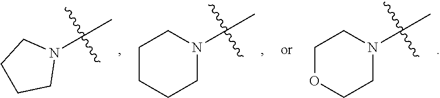 [SMILES: *N1CCCC1 ; *N1CCCCC1 ; *N1CCOCC1]

In some embodiments, R₃ is halo, OCH₃, OCF₃, OH, CN, or NO₂.

In some embodiments, each of Y₁, Y₂, and Y₃ is CR₃ and Y₄ is N, in which each R₃ is, e.g., independently, H or optionally substituted C₁-C₆ alkyl, e.g., H.

In some embodiments, each of Y₁, Y₂, and Y₄ is CR₃ and Y₃ is N, in which each R₃ is, e.g., H or optionally substituted C₁-C₆ alkyl, e.g., all H.

In some embodiments, Q₃ is optionally substituted C₁-C₄ heterocyclyl. For example, Q₃ is optionally substituted thiophene, optionally substituted pyrrole, optionally substituted furan, optionally substituted thiazole, optionally substituted oxazole, optionally substituted isothiazole, optionally substituted isooxazole, optionally substituted diazole, optionally substituted oxadiazole, optionally substituted thiadiazole, or optionally substituted triazole.

In some embodiments of any of the aspects described herein (e.g. the compound of formulas (II) and (IIA)), each of L₁ and L₂ is —(CH₂)₂—.

In some embodiments of any of the aspects described herein (e.g. the compound of formulas (II) and (IIA)), each of L₁ and L₂ is —CH₂—.

In some embodiments of any of the aspects described herein (e.g. the compound of formulas (II) and (IIA)), L₁ is —CH₂CF₂— and L₂ is —(CH₂)₂—.

In some embodiments of any of the aspects described herein (e.g. the compound of formulas (II) and (IIA)), an X₃ in L₁ and an X₃ in L₂ combine to form C₁-C₃ alkylene.

In some embodiments of any of the aspects described herein (e.g. the compound of formulas (II) and (IIA)), X₁ is O and X₂ is H.

In some embodiments of any of the aspects described herein (e.g. the compound of formulas (II) and (IIA)), X₁ is H and X₂ is O.

In some embodiments of any of the aspects described herein (e.g. the compound of formulas (I), (II), and (IIA)), each of R₁ and R₂ is optionally substituted C₆-C₁₆ aryl. In some embodiments, each of R₁ and R₂ is optionally substituted phenyl, preferably phenyl or 4-fluorophenyl. In some embodiments, R₁ is H, and R₂ is optionally substituted C₆-C₁₆ aryl, preferably optionally substituted phenyl, more preferably phenyl or 4-fluorophenyl.

In some embodiments, the compound is a compound of Table 1, or a pharmaceutically acceptable salt thereof:

TABLE 1

| Compound | Structure |
|---|---|
| 1 | 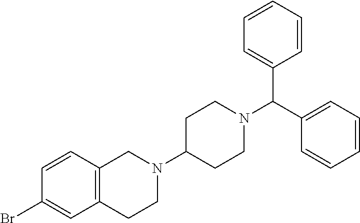 [SMILES: Brc1ccc2c(c1)CCN(C1CCN(C(c3ccccc3)c3ccccc3)CC1)C2] |
| 2 | 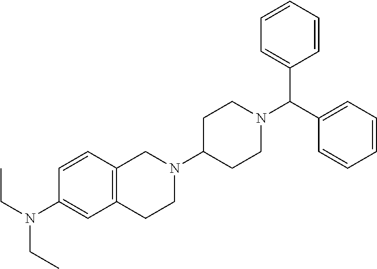 [SMILES: CCN(CC)c1ccc2c(c1)CCN(C1CCN(C(c3ccccc3)c3ccccc3)CC1)C2] |
| 3 | 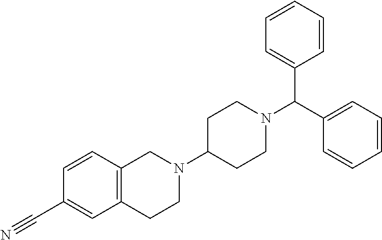 [SMILES: N#Cc1ccc2c(c1)CCN(C1CCN(C(c3ccccc3)c3ccccc3)CC1)C2] |
| 4 | 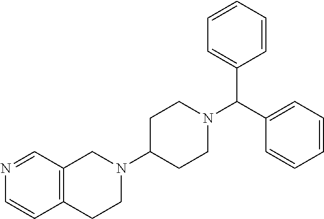 [SMILES: c1ccc(C(c2ccccc2)N2CCC(N3CCc4ccncc4C3)CC2)cc1] |
| 5 | 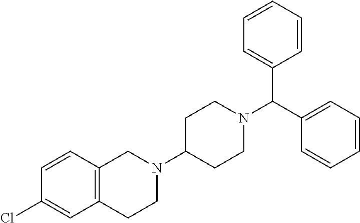 [SMILES: Clc1ccc2c(c1)CCN(C1CCN(C(c3ccccc3)c3ccccc3)CC1)C2] |
| 6 | 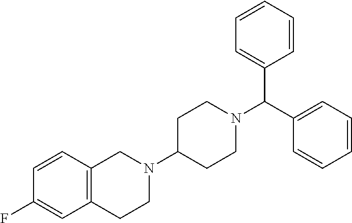 [SMILES: Fc1ccc2c(c1)CCN(C1CCN(C(c3ccccc3)c3ccccc3)CC1)C2] |
| 7 | 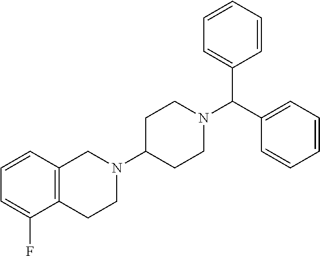 [SMILES: Fc1cccc2c1CCN(C1CCN(C(c3ccccc3)c3ccccc3)CC1)C2] |
| 8 | 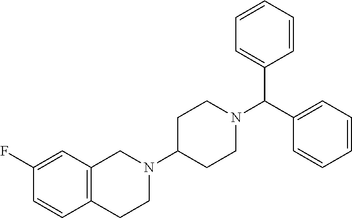 [SMILES: Fc1ccc2c(c1)CN(C1CCN(C(c3ccccc3)c3ccccc3)CC1)CC2] |
| 9 | 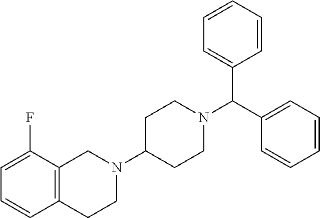 [SMILES: Fc1cccc2c1CN(C1CCN(C(c3ccccc3)c3ccccc3)CC1)CC2] |
| 10 | 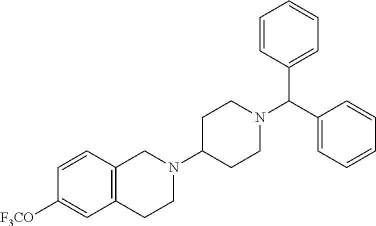 [SMILES: FC(F)(F)Oc1ccc2c(c1)CCN(C1CCN(C(c3ccccc3)c3ccccc3)CC1)C2] |
| 11 | 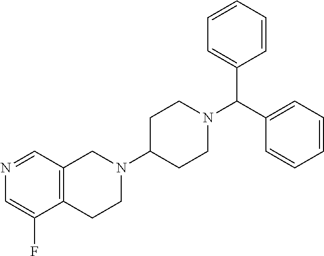 [SMILES: Fc1cncc2c1CCN(C1CCN(C(c3ccccc3)c3ccccc3)CC1)C2] |
| 12 | 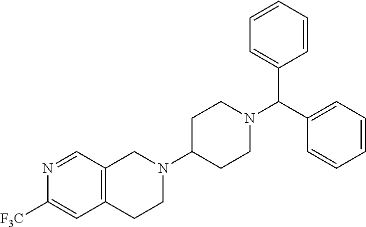 [SMILES: FC(F)(F)c1cc2c(cn1)CN(C1CCN(C(c3ccccc3)c3ccccc3)CC1)CC2] |
| 13 | 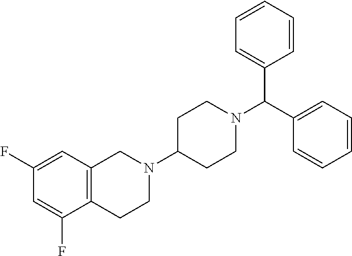 [SMILES: Fc1cc(F)c2c(c1)CN(C1CCN(C(c3ccccc3)c3ccccc3)CC1)CC2] |
| 14 | 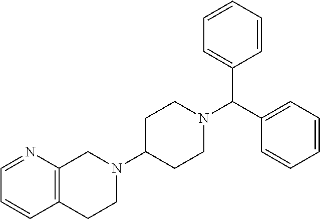 [SMILES: c1ccc(C(c2ccccc2)N2CCC(N3CCc4cccnc4C3)CC2)cc1] |
| 15 | 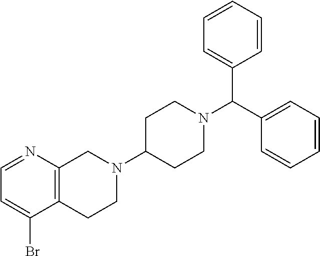 [SMILES: Brc1ccnc2c1CCN(C1CCN(C(c3ccccc3)c3ccccc3)CC1)C2] |
| 16 | 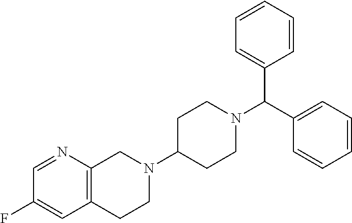 [SMILES: Fc1cnc2c(c1)CCN(C1CCN(C(c3ccccc3)c3ccccc3)CC1)C2] |
| 17 | 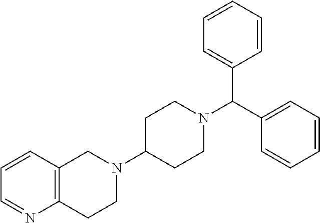 [SMILES: c1ccc(C(c2ccccc2)N2CCC(N3CCc4ncccc4C3)CC2)cc1] |
| 18 | 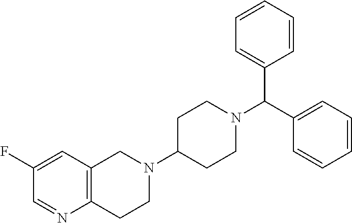 [SMILES: Fc1cnc2c(c1)CN(C1CCN(C(c3ccccc3)c3ccccc3)CC1)CC2] |
| 19 | 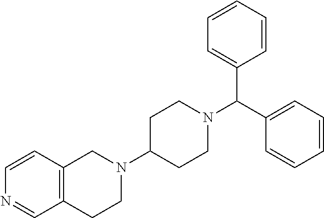 [SMILES: c1ccc(C(c2ccccc2)N2CCC(N3CCc4cnccc4C3)CC2)cc1] |
| 20 | 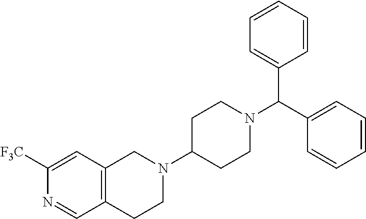 [SMILES: FC(F)(F)c1cc2c(cn1)CCN(C1CCN(C(c3ccccc3)c3ccccc3)CC1)C2] |
| 21 | 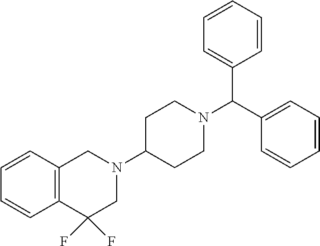 [SMILES: FC1(F)CN(C2CCN(C(c3ccccc3)c3ccccc3)CC2)Cc2ccccc21] |
| 22 | 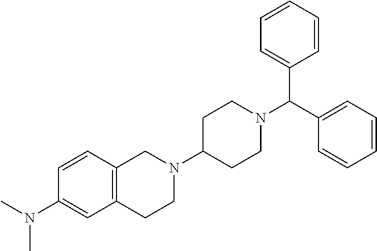 [SMILES: CN(C)c1ccc2c(c1)CCN(C1CCN(C(c3ccccc3)c3ccccc3)CC1)C2] |
| 23 | 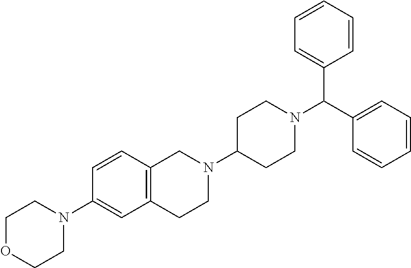 [SMILES: c1ccc(C(c2ccccc2)N2CCC(N3CCc4cc(N5CCOCC5)ccc4C3)CC2)cc1] |
| 24 | 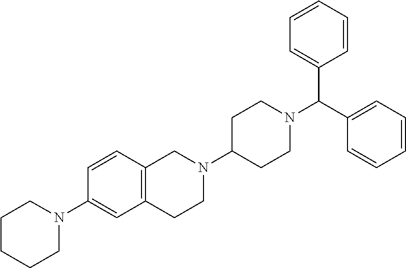 [SMILES: c1ccc(C(c2ccccc2)N2CCC(N3CCc4cc(N5CCCCC5)ccc4C3)CC2)cc1] |
| 25 | 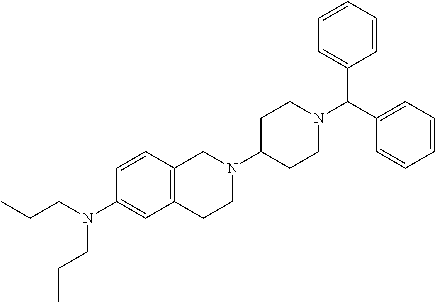 [SMILES: CCCN(CCC)c1ccc2c(c1)CCN(C1CCN(C(c3ccccc3)c3ccccc3)CC1)C2] |
| 26 | 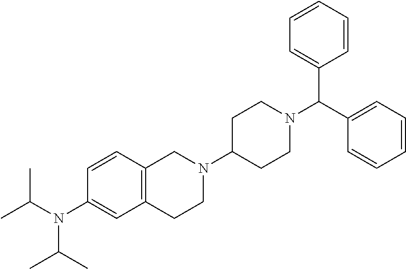 [SMILES: CC(C)N(c1ccc2c(c1)CCN(C1CCN(C(c3ccccc3)c3ccccc3)CC1)C2)C(C)C] |
| 27 | 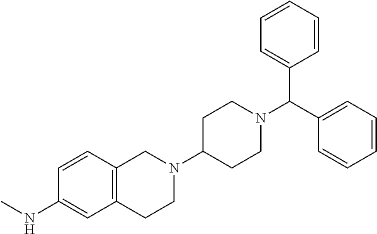 [SMILES: CNc1ccc2c(c1)CCN(C1CCN(C(c3ccccc3)c3ccccc3)CC1)C2] |
| 28 | 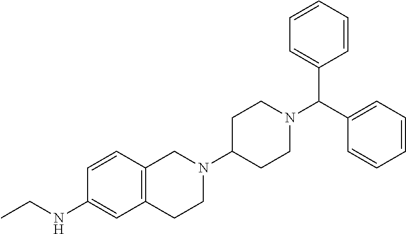 [SMILES: CCNc1ccc2c(c1)CCN(C1CCN(C(c3ccccc3)c3ccccc3)CC1)C2] |
| 29 | 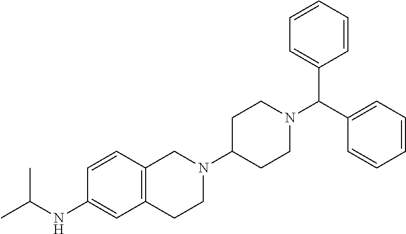 [SMILES: CC(C)Nc1ccc2c(c1)CCN(C1CCN(C(c3ccccc3)c3ccccc3)CC1)C2] |
| 30 | 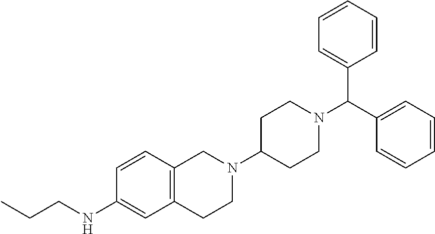 [SMILES: CCCNc1ccc2c(c1)CCN(C1CCN(C(c3ccccc3)c3ccccc3)CC1)C2] |
| 31 | 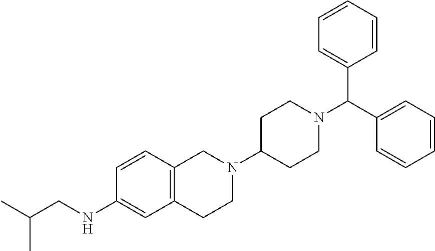 [SMILES: CC(C)CNc1ccc2c(c1)CCN(C1CCN(C(c3ccccc3)c3ccccc3)CC1)C2] |
| 32 | 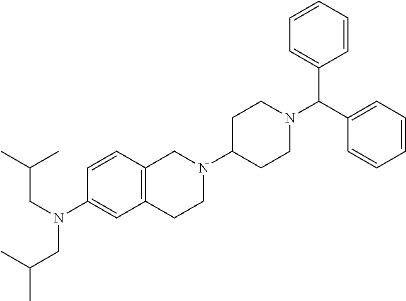 [SMILES: CC(C)CN(CC(C)C)c1ccc2c(c1)CCN(C1CCN(C(c3ccccc3)c3ccccc3)CC1)C2] |
| 33 | 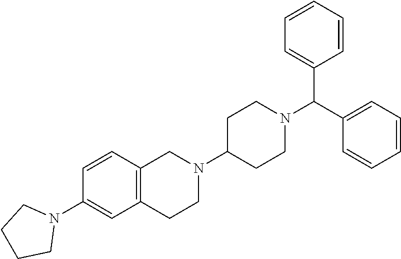 [SMILES: c1ccc(C(c2ccccc2)N2CCC(N3CCc4cc(N5CCCC5)ccc4C3)CC2)cc1] |
| 34 | 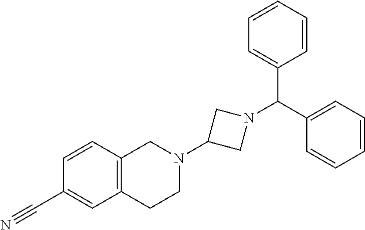 [SMILES: N#Cc1ccc2c(c1)CCN(C1CN(C(c3ccccc3)c3ccccc3)C1)C2] |
| 35 | 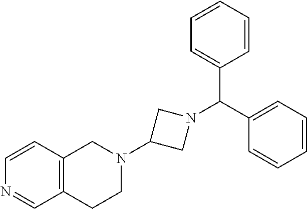 [SMILES: c1ccc(C(c2ccccc2)N2CC(N3CCc4cnccc4C3)C2)cc1] |
| 36 | 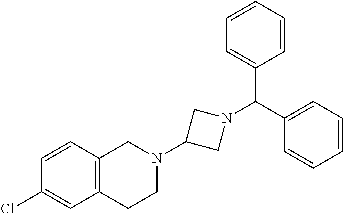 [SMILES: Clc1ccc2c(c1)CCN(C1CN(C(c3ccccc3)c3ccccc3)C1)C2] |
| 37 | 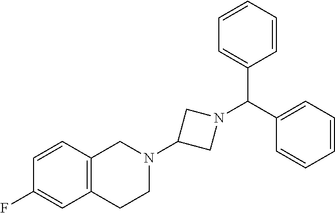 [SMILES: Fc1ccc2c(c1)CCN(C1CN(C(c3ccccc3)c3ccccc3)C1)C2] |
| 38 | 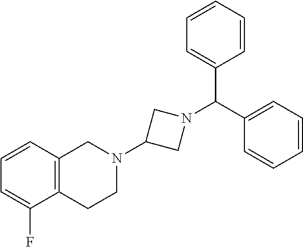 [SMILES: Fc1cccc2c1CCN(C1CN(C(c3ccccc3)c3ccccc3)C1)C2] |
| 39 | 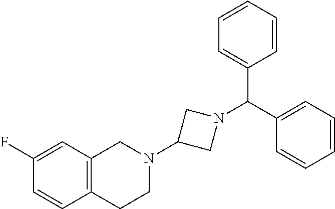 [SMILES: Fc1ccc2c(c1)CN(C1CN(C(c3ccccc3)c3ccccc3)C1)CC2] |
| 40 | 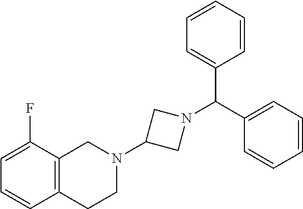 [SMILES: Fc1cccc2c1CN(C1CN(C(c3ccccc3)c3ccccc3)C1)CC2] |
| 41 | 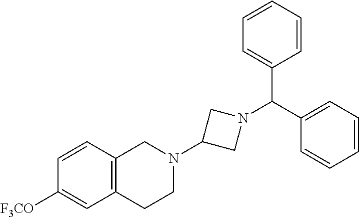 [SMILES: FC(F)(F)Oc1ccc2c(c1)CCN(C1CN(C(c3ccccc3)c3ccccc3)C1)C2] |
| 42 | 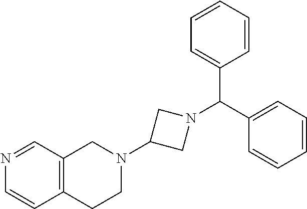 [SMILES: c1ccc(C(c2ccccc2)N2CC(N3CCc4ccncc4C3)C2)cc1] |
| 43 | 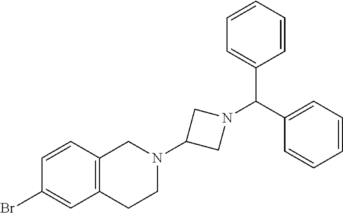 [SMILES: Brc1ccc2c(c1)CCN(C1CN(C(c3ccccc3)c3ccccc3)C1)C2] |
| 44 | 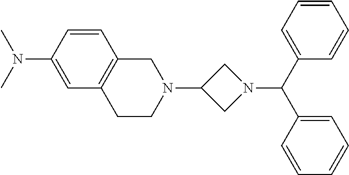 [SMILES: CN(C)c1ccc2c(c1)CCN(C1CN(C(c3ccccc3)c3ccccc3)C1)C2] |
| 45 | 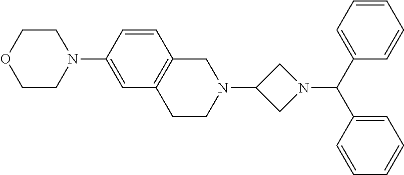 [SMILES: c1ccc(C(c2ccccc2)N2CC(N3CCc4cc(N5CCOCC5)ccc4C3)C2)cc1] |
| 46 | 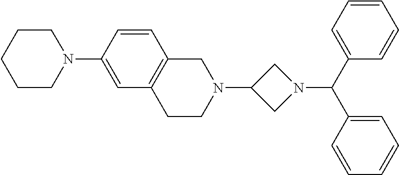 [SMILES: c1ccc(C(c2ccccc2)N2CC(N3CCc4cc(N5CCCCC5)ccc4C3)C2)cc1] |
| 47 | 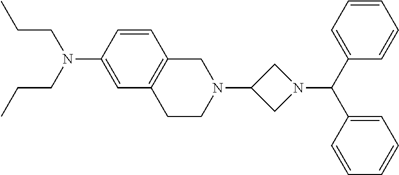 [SMILES: CCCN(CCC)c1ccc2c(c1)CCN(C1CN(C(c3ccccc3)c3ccccc3)C1)C2] |
| 48 | 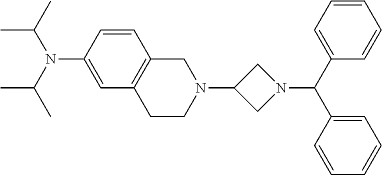 [SMILES: CC(C)N(c1ccc2c(c1)CCN(C1CN(C(c3ccccc3)c3ccccc3)C1)C2)C(C)C] |
| 49 | 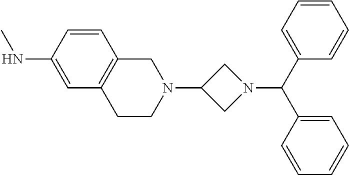 [SMILES: CNc1ccc2c(c1)CCN(C1CN(C(c3ccccc3)c3ccccc3)C1)C2] |
| 50 | 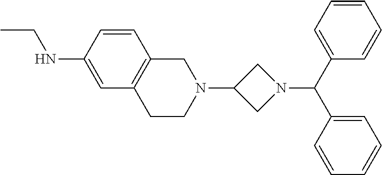 [SMILES: CCNc1ccc2c(c1)CCN(C1CN(C(c3ccccc3)c3ccccc3)C1)C2] |
| 51 | 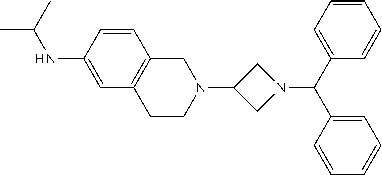 [SMILES: CC(C)Nc1ccc2c(c1)CCN(C1CN(C(c3ccccc3)c3ccccc3)C1)C2] |
| 52 | 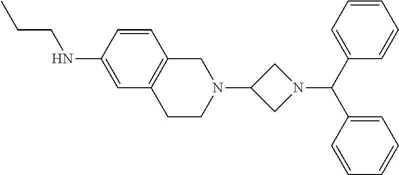 [SMILES: CCCNc1ccc2c(c1)CCN(C1CN(C(c3ccccc3)c3ccccc3)C1)C2] |
| 53 | 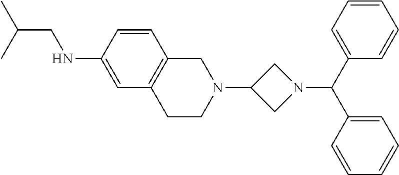 [SMILES: CC(C)CNc1ccc2c(c1)CCN(C1CN(C(c3ccccc3)c3ccccc3)C1)C2] |
| 54 | 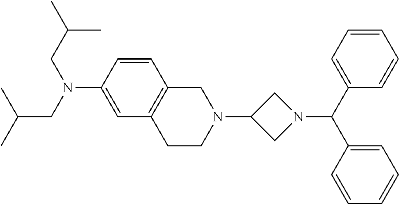 [SMILES: CC(C)CN(CC(C)C)c1ccc2c(c1)CCN(C1CN(C(c3ccccc3)c3ccccc3)C1)C2] |
| 55 | 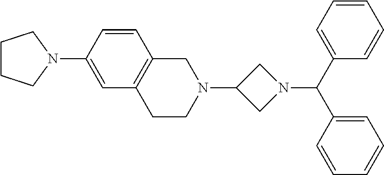 [SMILES: c1ccc(C(c2ccccc2)N2CC(N3CCc4cc(N5CCCC5)ccc4C3)C2)cc1] |
| 56 | 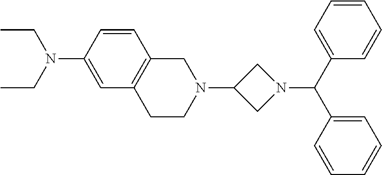 [SMILES: CCN(CC)c1ccc2c(c1)CCN(C1CN(C(c3ccccc3)c3ccccc3)C1)C2] |
| 57 | 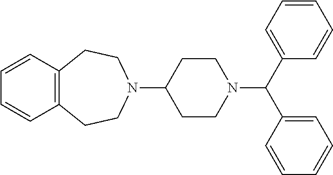 [SMILES: c1ccc(C(c2ccccc2)N2CCC(N3CCc4ccccc4CC3)CC2)cc1] |
| 58 | 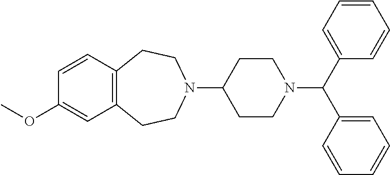 [SMILES: COc1ccc2c(c1)CCN(C1CCN(C(c3ccccc3)c3ccccc3)CC1)CC2] |
| 59 | 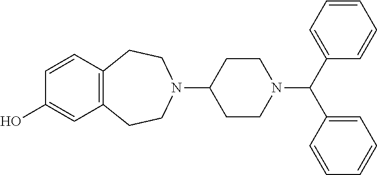 [SMILES: Oc1ccc2c(c1)CCN(C1CCN(C(c3ccccc3)c3ccccc3)CC1)CC2] |
| 60 | 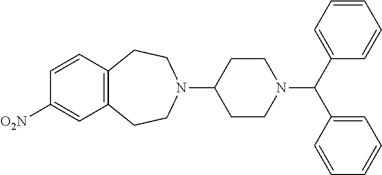 [SMILES: O=[N+]([O-])c1ccc2c(c1)CCN(C1CCN(C(c3ccccc3)c3ccccc3)CC1)CC2] |
| 61 | 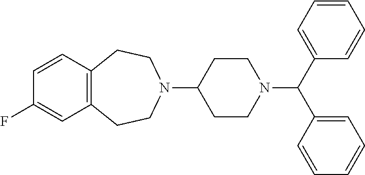 [SMILES: Fc1ccc2c(c1)CCN(C1CCN(C(c3ccccc3)c3ccccc3)CC1)CC2] |
| 62 | 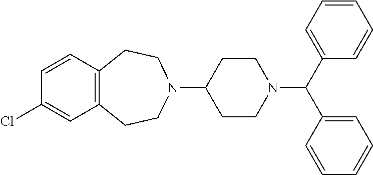 [SMILES: Clc1ccc2c(c1)CCN(C1CCN(C(c3ccccc3)c3ccccc3)CC1)CC2] |
| 63 | 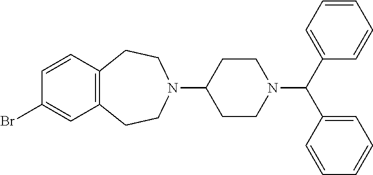 [SMILES: Brc1ccc2c(c1)CCN(C1CCN(C(c3ccccc3)c3ccccc3)CC1)CC2] |
| 64 | 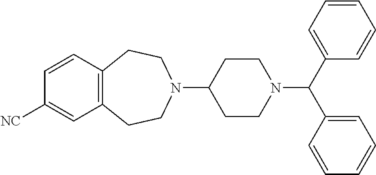 [SMILES: N#Cc1ccc2c(c1)CCN(C1CCN(C(c3ccccc3)c3ccccc3)CC1)CC2] |
| 65 | 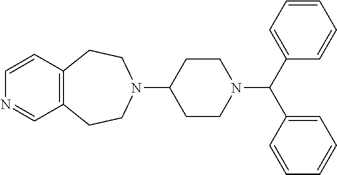 [SMILES: c1ccc(C(c2ccccc2)N2CCC(N3CCc4ccncc4CC3)CC2)cc1] |
| 66 | 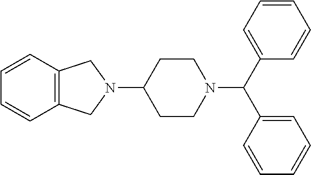 [SMILES: c1ccc(C(c2ccccc2)N2CCC(N3Cc4ccccc4C3)CC2)cc1] |
| 67 | 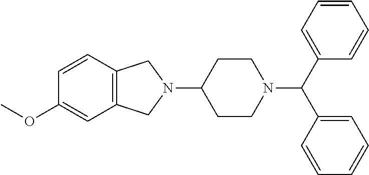 [SMILES: COc1ccc2c(c1)CN(C1CCN(C(c3ccccc3)c3ccccc3)CC1)C2] |
| 68 | 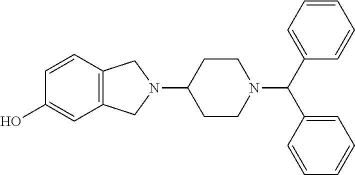 [SMILES: Oc1ccc2c(c1)CN(C1CCN(C(c3ccccc3)c3ccccc3)CC1)C2] |
| 69 | 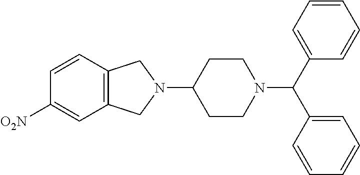 [SMILES: O=[N+]([O-])c1ccc2c(c1)CN(C1CCN(C(c3ccccc3)c3ccccc3)CC1)C2] |
| 70 | 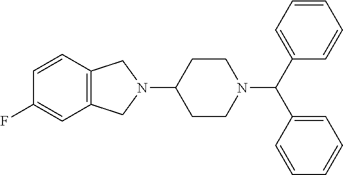 [SMILES: Fc1ccc2c(c1)CN(C1CCN(C(c3ccccc3)c3ccccc3)CC1)C2] |
| 71 | 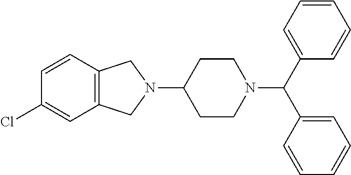 [SMILES: Clc1ccc2c(c1)CN(C1CCN(C(c3ccccc3)c3ccccc3)CC1)C2] |
| 72 | 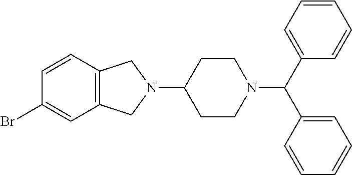 [SMILES: Brc1ccc2c(c1)CN(C1CCN(C(c3ccccc3)c3ccccc3)CC1)C2] |
| 73 | 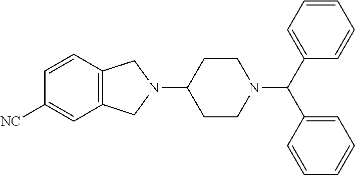 [SMILES: N#Cc1ccc2c(c1)CN(C1CCN(C(c3ccccc3)c3ccccc3)CC1)C2] |
| 74 | 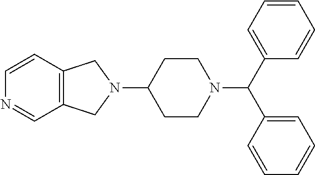 [SMILES: c1ccc(C(c2ccccc2)N2CCC(N3Cc4ccncc4C3)CC2)cc1] |
| 75 | 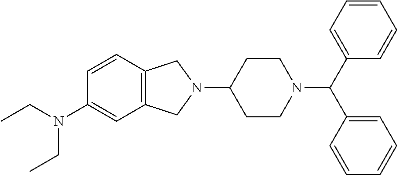 [SMILES: CCN(CC)c1ccc2c(c1)CN(C1CCN(C(c3ccccc3)c3ccccc3)CC1)C2] |
| 76 | 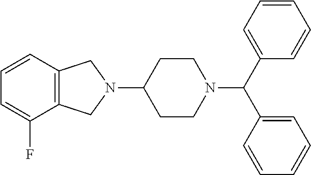 [SMILES: Fc1cccc2c1CN(C1CCN(C(c3ccccc3)c3ccccc3)CC1)C2] |
| 77 | 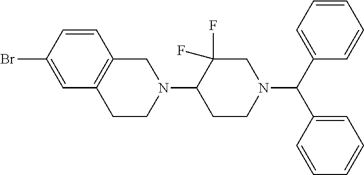 [SMILES: FC1(F)CN(C(c2ccccc2)c2ccccc2)CCC1N1CCc2cc(Br)ccc2C1] |
| 78 | 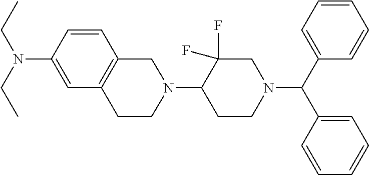 [SMILES: CCN(CC)c1ccc2c(c1)CCN(C1CCN(C(c3ccccc3)c3ccccc3)CC1(F)F)C2] |
| 79 | 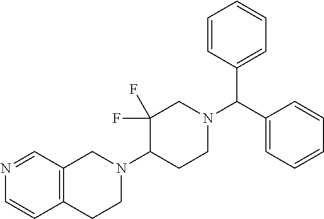 [SMILES: FC1(F)CN(C(c2ccccc2)c2ccccc2)CCC1N1CCc2ccncc2C1] |
| 80 | 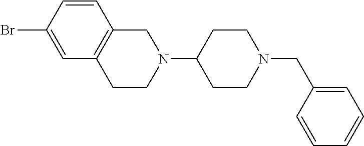 [SMILES: Brc1ccc2c(c1)CCN(C1CCN(Cc3ccccc3)CC1)C2] |
| 81 | 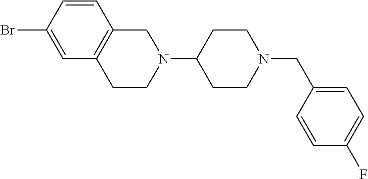 [SMILES: Fc1ccc(CN2CCC(N3CCc4cc(Br)ccc4C3)CC2)cc1] |
| 82 | 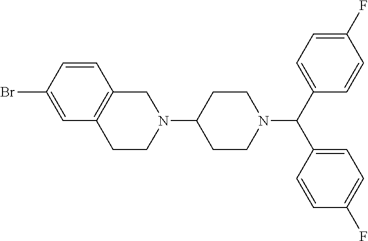 [SMILES: Fc1ccc(C(c2ccc(F)cc2)N2CCC(N3CCc4cc(Br)ccc4C3)CC2)cc1] |
| 83 | 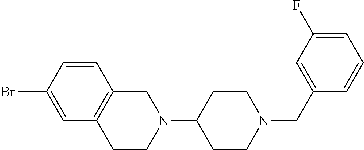 [SMILES: Fc1cccc(CN2CCC(N3CCc4cc(Br)ccc4C3)CC2)c1] |
| 84 | 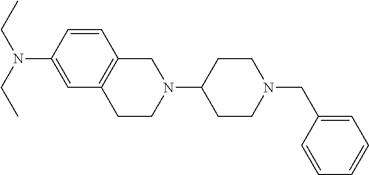 [SMILES: CCN(CC)c1ccc2c(c1)CCN(C1CCN(Cc3ccccc3)CC1)C2] |
| 85 | 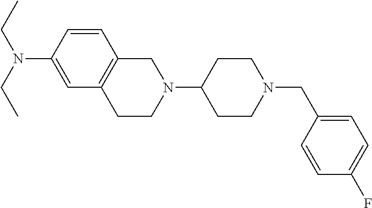 [SMILES: CCN(CC)c1ccc2c(c1)CCN(C1CCN(Cc3ccc(F)cc3)CC1)C2] |
| 86 | 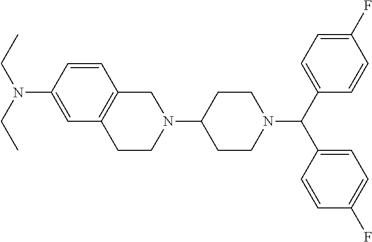 [SMILES: CCN(CC)c1ccc2c(c1)CCN(C1CCN(C(c3ccc(F)cc3)c3ccc(F)cc3)CC1)C2] |
| 87 | 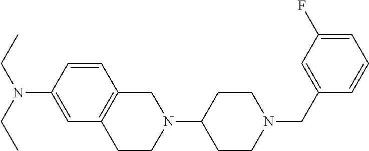 [SMILES: CCN(CC)c1ccc2c(c1)CCN(C1CCN(Cc3cccc(F)c3)CC1)C2] |
| 88 | 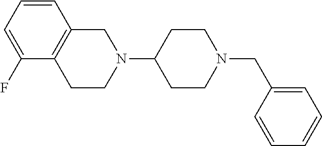 [SMILES: Fc1cccc2c1CCN(C1CCN(Cc3ccccc3)CC1)C2] |
| 89 | 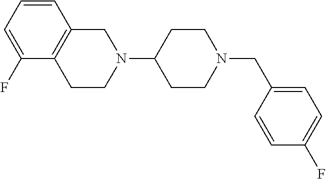 [SMILES: Fc1ccc(CN2CCC(N3CCc4c(F)cccc4C3)CC2)cc1] |
| 90 | 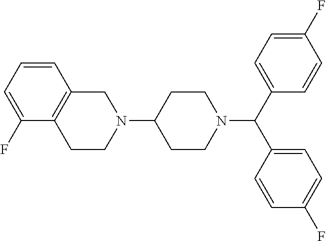 [SMILES: Fc1ccc(C(c2ccc(F)cc2)N2CCC(N3CCc4c(F)cccc4C3)CC2)cc1] |
| 91 | 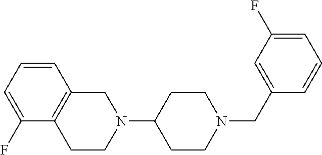 [SMILES: Fc1cccc(CN2CCC(N3CCc4c(F)cccc4C3)CC2)c1] |
| 92 | 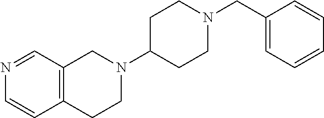 [SMILES: c1ccc(CN2CCC(N3CCc4ccncc4C3)CC2)cc1] |
| 93 | 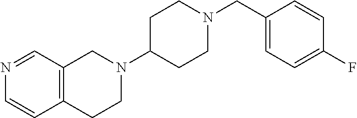 [SMILES: Fc1ccc(CN2CCC(N3CCc4ccncc4C3)CC2)cc1] |
| 94 | 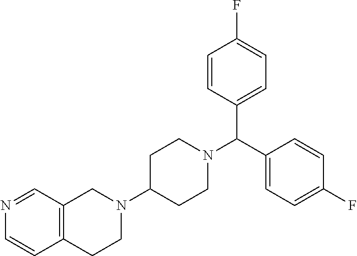 [SMILES: Fc1ccc(C(c2ccc(F)cc2)N2CCC(N3CCc4ccncc4C3)CC2)cc1] |
| 95 | 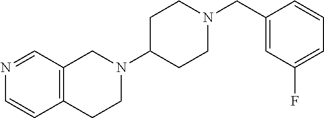 [SMILES: Fc1cccc(CN2CCC(N3CCc4ccncc4C3)CC2)c1] |
| 96 | 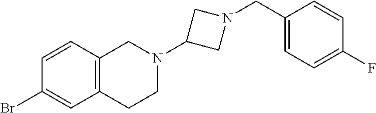 [SMILES: Fc1ccc(CN2CC(N3CCc4cc(Br)ccc4C3)C2)cc1] |
| 97 | 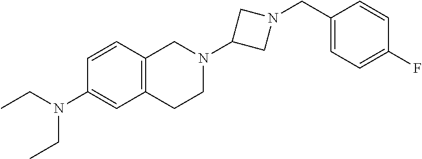 [SMILES: CCN(CC)c1ccc2c(c1)CCN(C1CN(Cc3ccc(F)cc3)C1)C2] |
| 98 | 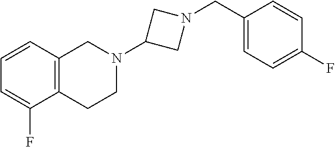 [SMILES: Fc1ccc(CN2CC(N3CCc4c(F)cccc4C3)C2)cc1] |
| 99 | 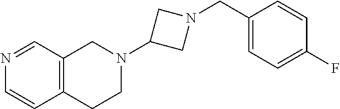 [SMILES: Fc1ccc(CN2CC(N3CCc4ccncc4C3)C2)cc1] |
| 100 | 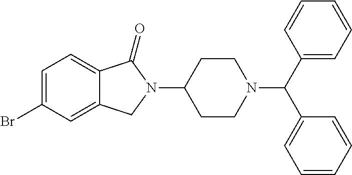 [SMILES: O=C1c2ccc(Br)cc2CN1C1CCN(C(c2ccccc2)c2ccccc2)CC1] |
| 101 | 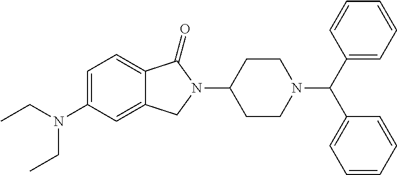 [SMILES: CCN(CC)c1ccc2c(c1)CN(C1CCN(C(c3ccccc3)c3ccccc3)CC1)C2=O] |
| 102 | 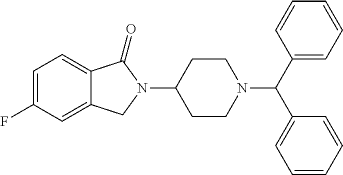 [SMILES: O=C1c2ccc(F)cc2CN1C1CCN(C(c2ccccc2)c2ccccc2)CC1] |
| 103 | 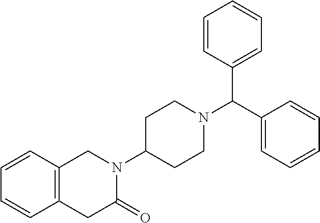 [SMILES: O=C1Cc2ccccc2CN1C1CCN(C(c2ccccc2)c2ccccc2)CC1] |
| 104 | 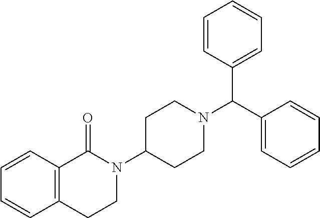 [SMILES: O=C1c2ccccc2CCN1C1CCN(C(c2ccccc2)c2ccccc2)CC1] |
| 105 | 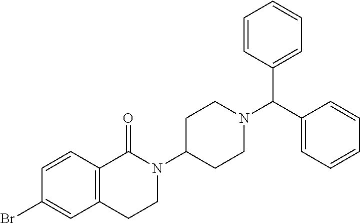 [SMILES: O=C1c2ccc(Br)cc2CCN1C1CCN(C(c2ccccc2)c2ccccc2)CC1] |
| 106 | 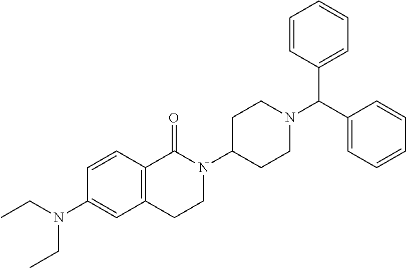 [SMILES: CCN(CC)c1ccc2c(c1)CCN(C1CCN(C(c3ccccc3)c3ccccc3)CC1)C2=O] |

In some embodiments, the compound is Compound 2:

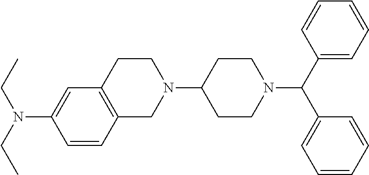 [SMILES: CCN(CC)c1ccc2c(c1)CCN(C1CCN(C(c3ccccc3)c3ccccc3)CC1)C2]

or a pharmaceutically acceptable salt thereof.

In some embodiments, the compound is Compound 4:

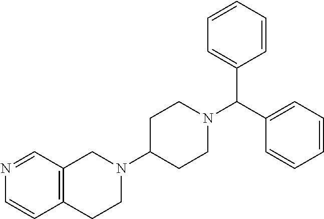 [SMILES: c1ccc(C(c2ccccc2)N2CCC(N3CCc4ccncc4C3)CC2)cc1]

or a pharmaceutically acceptable salt thereof.

In some embodiments, the compound is Compound 7:

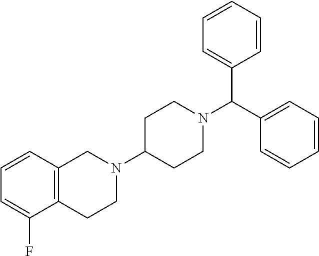 [SMILES: Fc1cccc2c1CCN(C1CCN(C(c3ccccc3)c3ccccc3)CC1)C2]

or a pharmaceutically acceptable salt thereof.

In some embodiments, the compound is Compound 43:

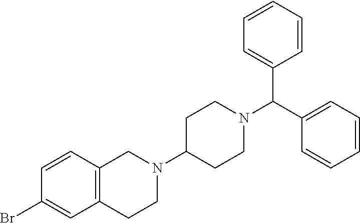 [SMILES: Brc1ccc2c(c1)CCN(C1CCN(C(c3ccccc3)c3ccccc3)CC1)C2]

or a pharmaceutically acceptable salt thereof.

In some embodiments, the compound is Compound 85:

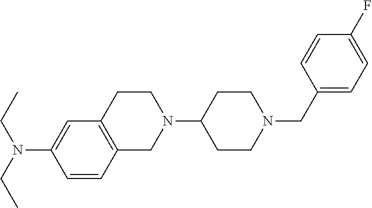 [SMILES: CCN(CC)c1ccc2c(c1)CCN(C1CCN(Cc3ccc(F)cc3)CC1)C2]

or a pharmaceutically acceptable salt thereof.

In another aspect, the invention features a pharmaceutical composition including a compound of the invention (e.g., a compound of any one of formulas (I), (II), and (IIA) or any one of the compounds of Table 1), or a pharmaceutically acceptable salt thereof, and a pharmaceutically acceptable excipient.

In another aspect, the invention features a method of treating a subject having a disease or injury. The method includes administering to the subject a therapeutically effective amount of a compound of the invention (e.g., a compound of any one of formulas (I), (II), and (IIA) or any one of the compounds of Table 1), or a pharmaceutically acceptable salt thereof, or a pharmaceutical composition of the invention.

In some embodiments, the disease or injury is stroke, e.g., acute stroke and/or stroke in a recovery phase; congenital hypogonadotropic hypogonadism (e.g., Kallmann Syndrome); cerebral hemorrhage; traumatic brain injury (TBI); spinal cord injury (SCI); peripheral vascular disease (PVD); wounds, i.e., for wound healing; bone or cartilage injury; hearing loss; depression; anxiety; post-traumatic stress disorder (PTSD); substance abuse; peripheral nerve injury; hematopoietic disorders; amyotrophic lateral sclerosis (ALS); Alzheimer's disease; Parkinson's disease; heart disease; non-arteritic ischemic optic neuropathy (NAION); retinal artery occlusion; bronchopulmonary dysplasia, muscular dystrophy, anosmia, aging, memory disturbance, or viral infection. In certain embodiments, the disease or injury is stroke, e.g., acute stroke and/or stroke in a recovery phase. In other embodiments, the disease or injury is congenital hypogonadotropic hypogonadism, e.g., Kallmann Syndrome. In other embodiments, the disease or injury is viral infection.

In another aspect, the invention features a method of increasing spermatogenesis in a subject. The method includes administering to the subject an effective amount of a compound of the invention (e.g., a compound of any one of formulas (I), (II), and (IIA) or any one of the compounds of Table 1), or a pharmaceutical acceptable salt thereof, or a pharmaceutical composition of the invention.

### Definitions

To facilitate the understanding of this invention, a number of terms are defined below. Terms defined herein have meanings as commonly understood by a person of ordinary skill in the areas relevant to the invention. Terms such as “a”, “an,” and “the” are not intended to refer to only a singular entity, but include the general class of which a specific example may be used for illustration. The terminology herein is used to describe specific embodiments of the invention, but their usage does not limit the invention, except as outlined in the claims.

As used herein, the term “about” refers to a value that is within 10% above or below the value being described.

As used herein, any values provided in a range of values include both the upper and lower bounds, and any values contained within the upper and lower bounds.

As used herein, the term “pharmaceutically acceptable salt” represents those salts of the compounds described that are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Handbook of Pharmaceutical Salts: Properties, Selection, and Use, (Eds. P. H. Stahl and C. G. Wermuth), Wiley-VCH, 2008. These salts may be acid addition salts involving inorganic or organic acids. The salts can be prepared in situ during the final isolation and purification of the compounds described herein or separately by reacting the free base group with a suitable acid. Methods for preparation of the appropriate salts are well-established in the art. Representative acid addition salts include acetate, adipate, alginate, ascorbate, aspartate, benzenesulfonate, benzoate, bisulfate, borate, bromide, butyrate, camphorate, camphorsulfonate, chloride, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like.

As used herein, the term “therapeutically effective amount” refers to an amount sufficient to effect beneficial or desired results, such as clinical results, and, as such, a “therapeutically effective amount” depends upon the context in which it is being applied. For example, in the context of administering a compound of formula (I), e.g., any one of Compounds 1-106, to treat or enhance a subject's recovery from a stroke or TBI, a therapeutically effective amount of a compound is, for example, an amount sufficient to alleviate or reverse the effect of the stroke or TBI. For example, the subject may regain lost motor functions due to the stroke or TBI.

As used herein, and as well understood in the art, “to treat” a condition or “treatment” of various diseases and disorders is an approach for obtaining beneficial or desired results, such as clinical results. Beneficial or desired results can include, but are not limited to, alleviation of one or more symptoms or conditions; diminishment of extent of disease, disorder, or condition; stabilizing (i.e., not worsening) state of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

The term “subject,” as used herein, can be a human, non-human primate, or other mammal, such as but not limited to dog, cat, horse, cow, pig, goat, monkey, rat, mouse, and sheep.

As used herein, the term “pharmaceutical composition” refers to an active compound, formulated together with one or more pharmaceutically acceptable excipients. In some embodiments, a compound of the invention, e.g., any one of Compounds 1-106, is present in unit dose amount appropriate for administration in a therapeutic regimen that shows a statistically significant probability of achieving a predetermined therapeutic effect when administered to a relevant population. In certain embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

The term “pharmaceutically acceptable excipient,” as used herein, refers to any inactive ingredient (for example, a vehicle capable of suspending or dissolving the active compound) having the properties of being nontoxic and non-inflammatory in a subject. Typical excipients include, for example: antiadherents, antioxidants, binders, coatings, compression aids, disintegrants, dyes, emollients, emulsifiers, diluents, film formers or coatings, flavors, fragrances, glidants, lubricants, preservatives, printing inks, sorbents, suspending or dispersing agents, sweeteners, or waters of hydration. Excipients include, but are not limited to: butylated optionally substituted hydroxytoluene (e.g., BHT), calcium carbonate, calcium phosphate dibasic, calcium stearate, croscarmellose, crosslinked polyvinyl pyrrolidone, citric acid, crospovidone, cysteine, ethylcellulose, gelatin, optionally substituted hydroxypropyl cellulose, optionally substituted hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch, stearic acid, stearic acid, sucrose, talc, titanium dioxide, vitamin A, vitamin E, vitamin C, and xylitol. Those of ordinary skill in the art are familiar with a variety of agents and materials useful as excipients.

The term “alkyl,” as used herein, refers to a branched or straight-chain monovalent saturated aliphatic radical containing only C and H when unsubstituted. The monovalency of an alkyl group does not include the optional substituents on the alkyl group. For example, if an alkyl group is attached to a compound, monovalency of the alkyl group refers to its attachment to the compound and does not include any additional substituents that may be present on the alkyl group. In some embodiments, the alkyl group may contain, e.g., 1-20, 1-18, 1-16, 1-14, 1-12, 1-10, 1-8, 1-6, 1-4, or 1-2 carbon atoms (e.g., C₁-C₂₀, C₁-C₁₈, C₁-C₁₆, C₁-C₁₄, C₁-C₁₂, C₁-C₁₀, C₁-C₈, C₁-C₆, C₁-C₄, or C₁-C₂). Examples include, but are not limited to, methyl, ethyl, isobutyl, sec-butyl, and tert-butyl.

The term “alkylene,” as used herein, refers to a divalent radical obtained by removing a hydrogen atom from a carbon atom of an alkyl group. The divalency of an alkylene group does not include the optional substituents on the alkylene group. Examples of alkylene groups include, but are not limited to, methylene, ethylene, and n-propylene.

The term “alkenyl,” as used herein, refers to a branched or straight-chain monovalent unsaturated aliphatic radical containing at least one carbon-carbon double bond and no carbon-carbon triple bonds, and only C and H when unsubstituted. Monovalency of an alkenyl group does not include the optional substituents on the alkenyl group. For example, if an alkenyl group is attached to a compound, monovalency of the alkenyl group refers to its attachment to the compound and does not include any additional substituents that may be present on the alkenyl group. In some embodiments, the alkenyl group may contain, e.g., 2-20, 2-18, 2-16, 2-14, 2-12, 2-10, 2-8, 2-6, or 2-4 carbon atoms (e.g., C₂-C₂₀, C₂-C₁₈, C₂-C₁₆, C₂-C₁₄, C₂-C₁₂, C₂-C₁₀, C₂-C₈, C₂-C₆, or C₂-C₄). Examples include, but are not limited to, ethenyl, 1-propenyl, 2-propenyl, 1-methylethenyl, 1-butenyl, 2-butenyl, 3-butenyl, and the like.

The term “alkynyl,” as used herein, refers to a branched or straight-chain monovalent unsaturated aliphatic radical containing at least one carbon-carbon triple bond and only C and H when unsubstituted. Monovalency of an alkynyl group does not include the optional substituents on the alkynyl group. For example, if an alkynyl group is attached to a compound, monovalency of the alkynyl group refers to its attachment to the compound and does not include any additional substituents that may be present on the alkynyl group. In some embodiments, the alkynyl group may contain, e.g., 2-20, 2-18, 2-16, 2-14, 2-12, 2-10, 2-8, 2-6, or 2-4 carbon atoms (e.g., C₂-C₂₀, C₂-C₁₈, C₂-C₁₆, C₂-C₁₄, C₂-C₁₂, C₂-C₁₀, C₂-C₈, C₂-C₆, or C₂-C₄). Examples include, but are not limited to, ethynyl, 1-propynyl, and 3-butynyl.

The term “aryl,” as used herein, refers to any monocyclic or fused ring bicyclic or multicyclic system containing only carbon atoms in the ring(s), which has the characteristics of aromaticity in terms of electron distribution throughout the ring system, e.g., phenyl, naphthyl, or phenanthryl. An aryl group may have, e.g., six to sixteen carbons (e.g., six carbons, ten carbons, thirteen carbons, fourteen carbons, or sixteen carbons).

The term “cycloalkyl,” as used herein, represents a monovalent, saturated cyclic group containing only C and H when unsubstituted. A cycloalkyl may have, e.g., three to twenty carbons (e.g., a C₃-C₇, C₃-C₈, C₃-C₉, C₃-C₁₀, C₃-C₁₁, C₃-C₁₂, C₃-C₁₄, C₃-C₁₆, C₃-C₁₈, or C₃-C₂₀ cycloalkyl). Examples of cycloalkyls include, but are not limited to, cyclopropyl, cyclobutyl, cyclopentyl, cyclohexyl, and cycloheptyl. The term “cycloalkyl” also includes cyclic groups having a bridged multicyclic structure in which one or more carbons bridges two non-adjacent members of a monocyclic ring, e.g., bicyclo[2.2.1]heptyl and adamantyl. The term “cycloalkyl” also includes bicyclic, tricyclic, and tetracyclic fused ring structures, e.g., decalin and spiro-cyclic compounds.

The term “cycloalkenyl,” as used herein, represents a monovalent, unsaturated carbocyclic group that includes at least one carbon-carbon double bond, and only C and H when unsubstituted, and is not fully aromatic. A cycloalkenyl may have, e.g., four to twenty carbons (e.g., a C₄-C₇, C₄-C₈, C₄-C₉, C₄-C₁₀, C₄-C₁₁, C₄-C₁₂, C₄-C₁₃, C₄-C₁₄, C₄-C₁₆, C₄-C₁₈, or C₄-C₂₀ cycloalkenyl). Exemplary cycloalkenyl groups include, but are not limited to, cyclopentenyl, cyclohexenyl, and cycloheptenyl. The term “cycloalkenyl” also includes cyclic groups having a bridged multicyclic structure in which one or more carbons bridges two non-adjacent members of a monocyclic ring, e.g., bicyclo[2.2.2]oct-2-ene. The term “cycloalkenyl” also includes fused bicyclic and multicyclic nonaromatic, carbocyclic ring systems containing one or more double bonds, e.g., fluorene.

The term “halo,” as used herein, refers to a fluorine (fluoro), chlorine (chloro), bromine (bromo), or iodine (iodo) radical.

The term “heterocyclyl,” as used herein, represents a monocyclic or fused ring bicyclic or multicyclic system having at least one heteroatom as a ring atom. For example, a heterocyclyl ring may have, e.g., one to fifteen carbons ring atoms (e.g., a C₁-C₂, C₁-C₃, C₁-C₄, C₁-C₅, C₁-C₆, C₁-C₇, C₁-C₈, C₁-C₉, C₁-C₁₀, C₁-C₁₁, C₁-C₁₂, C₁-C₁₃, C₁-C₁₄, or C₁-C₁₅ heterocyclyl) and one or more (e.g., one, two, three, four, or five) ring heteroatoms independently selected from the group consisting of nitrogen, oxygen, and sulfur. Heterocyclyl groups may or may not include a ring that is aromatic. In preferred embodiments of the invention, a heterocyclyl group is a 3- to 8-membered ring, a 3- to 6-membered ring, a 4- to 6-membered ring, most preferably a 5-membered ring or a 6-membered ring. Exemplary 5-membered heterocyclyl groups may have zero to two double bonds, and exemplary 6-membered heterocyclyl groups may have zero to three double bonds. Exemplary 5-membered groups include, for example, optionally substituted pyrrole, optionally substituted pyrazole, optionally substituted isoxazole, optionally substituted pyrrolidine, optionally substituted imidazole, optionally substituted thiazole, optionally substituted thiophene, optionally substituted thiolane, optionally substituted furan, optionally substituted tetrahydrofuran, optionally substituted diazole, optionally substituted triazole, optionally substituted tetrazole, optionally substituted oxazole, optionally substituted 1,3,4-oxadiazole, optionally substituted 1,3,4-thiadiazole, optionally substituted 1,2,3,4-oxatriazole, and optionally substituted 1,2,3,4-thiatriazole. Exemplary 6-membered heterocyclyl groups include, for example, optionally substituted pyridine, optionally substituted piperidine, optionally substituted piperazine, optionally substituted pyrimidine, optionally substituted pyrazine, optionally substituted pyridazine, optionally substituted triazine, optionally substituted 2H-pyran, optionally substituted 4H-pyran, and optionally substituted tetrahydropyran. Exemplary 7-membered heterocyclyl groups include optionally substituted azepine, optionally substituted 1,4-diazepine, optionally substituted thiepine, and optionally substituted 1,4-thiazepine.

The term “heterocyclylene,” as used herein, refers to a divalent radical obtained by removing a hydrogen from a ring atom from a heterocyclyl group. The divalency of a heterocyclylene group does not include the optional substituents on the heterocyclylene group.

The term “oxo,” as used herein, refers to a divalent oxygen atom represented by the structure═O.

The phrase “optionally substituted X,” as used herein, is intended to be equivalent to “X, wherein X is optionally substituted” (e.g., “alkyl, wherein said alkyl is optionally substituted”). It is not intended to mean that the feature “X” (e.g. alkyl) per se is optional. The term “optionally substituted,” as used herein, refers to having 0, 1, or more substituents (e.g., 0-25, 0-20, 0-10, or 0-5 substituents).

Alkyl, alkylene, alkenyl, alkynyl, cycloalkyl, cycloalkenyl, aryl, heterocyclyl, and heterocyclylene groups may be substituted with cycloalkyl; cycloalkenyl; aryl; heterocyclyl; halo; OR^{a}, wherein Ra is H, alkyl, alkenyl, alkynyl, cycloalkyl, cycloalkenyl, aryl, or heterocyclyl; SR^{a}, wherein Ra is as defined herein; CN; NO₂; N₃; NR^{b}R^{c}; wherein each of R^{b} and RC is, independently, H, alkyl, alkenyl, alkynyl, cycloalkyl, cycloalkenyl, aryl, or heterocyclyl; SO₂Rd, wherein R^{d} is H, alkyl or aryl; SO₂NR^{e}R^{f}, wherein each of Re and R^{f} is, independently, H, alkyl, or aryl; SOR9, wherein R₉ is H, alkyl, or aryl; or SiR^{h}Rⁱ, wherein R^{h} and Rⁱ is, independently, H or alkyl. Aryl, cycloalkyl, cycloalkenyl, heteroaryl, and heterocyclyl groups may also be substituted with alkyl, alkenyl, or alkynyl. Alkyl, alkylene, alkenyl, alkynyl, cycloalkyl, cycloalkenyl, heterocyclyl, and heterocyclylene groups may also be substituted with oxo or ═NR^{j}, wherein Rⁱ is H or alkyl. In some embodiments, a substituent is further substituted as described herein. For example, a C₁ alkyl group, i.e., methyl, may be substituted with oxo to form a formyl group and further substituted with —OH or —NH₂ to form a carboxyl group or an amido group.

### DESCRIPTION OF THE DRAWINGS

FIG. 1 is a graph showing the thermal stability assay (TSA) data of purified FGF-2.FGFR1 complex with and without the addition of Compound 2 (dotted line: without Compound 2; solid line: 10 μM Compound 2).

FIG. 2 is a graph showing the phosphorylation of FGFR1 in the presence of increasing concentrations of Compound 2 in a cell-based system.

FIG. 3 is a graph showing the behavioral score of rats in a forelimb placing test pre-middle cerebral artery occlusion (MCAO) and post-MCAO (treated with Compound 2 or vehicle).

FIG. 4 is a graph showing the behavioral score of rats in a hindlimb placing test pre-MCAO and post-MCAO (treated with Compound 2 or vehicle).

FIG. 5 is a graph showing the right swing % of rats in a body swing test pre-MCAO and post-MCAO (treated with Compound 2 or vehicle).

FIG. 6 is a graph showing the body weight of rats pre-MCAO and post-MCAO (treated with Compound 2 or vehicle).

### DETAILED DESCRIPTION OF THE INVENTION

The invention features compounds, compositions, and methods for treating various diseases, disorders, and other medical conditions, for example, stroke, e.g., acute stroke and/or stroke in a recovery phase; congenital hypogonadotropic hypogonadism (e.g., Kallmann Syndrome); cerebral hemorrhage; traumatic brain injury (TBI); spinal cord injury (SCI); peripheral vascular disease (PVD); wounds, i.e., for wound healing; bone or cartilage injury; hearing loss; depression; anxiety; post-traumatic stress disorder (PTSD); substance abuse; peripheral nerve injury; hematopoietic disorders; amyotrophic lateral sclerosis (ALS); Alzheimer's disease; Parkinson's disease; heart disease; non-arteritic ischemic optic neuropathy (NAION); retinal artery occlusion; bronchopulmonary dysplasia, muscular dystrophy, anosmia, aging, memory disturbance, or viral infection, by administering a compound of formula (I) described herein, e.g., any one of Compounds 1-106, to the subject. Without wishing to be bound by theory, the compounds are believed to modulate FGF activity, e.g., by enhancing the binding between FGF-2 and its receptors, e.g., FGF-R₁. Preferably, methods of the invention are directed to enhancing a subject's recovery from brain injuries and diseases, such as cerebrovascular diseases, e.g., stroke (such as stroke recovery) and TBI.

### Compounds

The compounds provided herein include compounds of formula (I):

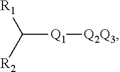

or pharmaceutically acceptable salts thereof, wherein R₁ is H, optionally substituted C₃-C₂₀ cycloalkyl, optionally substituted C₄-C₂₀ cycloalkenyl, optionally substituted C₁-C₁₅ heterocyclyl, or optionally substituted C₆-C₁₆ aryl; R₂ is optionally substituted C₃-C₂₀ cycloalkyl, optionally substituted C₄-C₂₀ cycloalkenyl, optionally substituted C₁-C₁₅ heterocyclyl, or optionally substituted C₆-C₁₆ aryl; Q₁ is optionally substituted 4-to-6 membered heterocyclylene containing at least one nitrogen atom; Q₂ is optionally substituted 5-to-7 membered heterocyclyl containing at least one nitrogen atom; and Q₃ is optionally substituted C₁-C₁₅ heterocyclyl, optionally substituted C₆-C₁₆ aryl, optionally substituted C₃-C₂₀ cycloalkyl, or optionally substituted C₄-C₂₀ cycloalkenyl, wherein Q₃ is fused to Q₂.

In some embodiments, the compounds have the structure of formula (II):

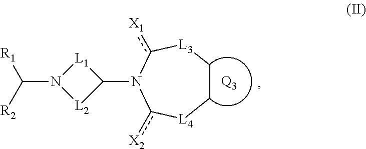

or pharmaceutically acceptable salts thereof, wherein L₁ and L₂ are each independently —C(X₃)₂— or —(C(X₃)₂)₂—, wherein each X₃ is independently H, halo, CN, NO₂, or C₁-C₆ alkyl; or an X₃ in L₁ and an X₃ in L₂ combine to form 01-O₃ alkylene; each of L₃ and L₄ is independently absent or C(X₄)₂—, wherein each X₄ is independently H, halo, CN, NO₂, or C₁-C₆ alkyl; each [symbol] is independently a single or double bond; and each of X₁ and X₂ is independently O when [symbol] is a double bond or H when [symbol] is a single bond.

Exemplary compounds of this invention are shown in Table 1 above.

### Pharmaceutical Compositions

A pharmaceutical composition of the invention contains one or more compounds of formula (I), e.g., Compounds 1-106, as the therapeutic compound. In addition to a therapeutically effective amount of the compound, the pharmaceutical compositions also contain a pharmaceutically acceptable excipient, which can be formulated by methods known to those skilled in the art. In some embodiments, pharmaceutical compositions of the invention contain one or more compounds of formula (I), e.g., any one of Compounds 1-106, and one or more exogenous ligands, e.g., exogenous FGF-2. The compounds of formula (I), e.g., any one of Compounds 1-106, may also be administered with or without other therapeutics for a particular condition.

The compounds of formula (I), e.g., any one of Compounds 1-106, may be used in the form of free base, or in the form of salts, solvates, and as prodrugs. All forms are within the scope of the invention.

Exemplary routes of administration of the pharmaceutical compositions (or the compounds of the composition) include oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, and topical administration.

Formulations for Oral Administration

The pharmaceutical compositions of the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Pharmaceutical compositions for oral administration may also be presented as chewable tablets, as hard gelatin capsules where the active ingredient is mixed with an inert solid diluent (e.g., potato starch, lactose, microcrystalline cellulose, calcium carbonate, calcium phosphate or kaolin), or as soft gelatin capsules where the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration versus time profile. In one example, controlled release is obtained by appropriate selection of various formulation parameters and ingredients, including, e.g., various types of controlled release compositions and coatings. Examples include single or multiple unit tablet or capsule compositions, oil solutions, suspensions, emulsions, microcapsules, microspheres, nanoparticles, patches, and liposomes. In some embodiments, compositions include biodegradable, pH, and/or temperature-sensitive polymer coatings.

Dissolution or diffusion-controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils, e.g., cottonseed oil, sesame oil, coconut oil, or peanut oil, as well as elixirs and similar pharmaceutical vehicles.

Formulations for Parenteral Administration

The pharmaceutical compositions of the invention can be administered in a pharmaceutically acceptable parenteral (e.g., intravenous, intramuscular, subcutaneous or the like) formulation as described herein. The pharmaceutical composition may also be administered parenterally in dosage forms or formulations containing conventional, non-toxic pharmaceutically acceptable carriers and adjuvants. In particular, formulations suitable for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. For example, to prepare such a composition, the compounds of the invention may be dissolved or suspended in a parenterally acceptable liquid vehicle. Among acceptable vehicles and solvents that may be employed are water; water adjusted to a suitable pH by addition of an appropriate amount of hydrochloric acid, sodium hydroxide, or a suitable buffer; 1,3-butanediol; Ringer's solution; and isotonic sodium chloride solution. The aqueous formulation may also contain one or more preservatives, for example, methyl, ethyl, or n-propyl p-hydroxybenzoate. Additional information regarding parenteral formulations can be found, for example, in the United States Pharmacopeia-National Formulary (USP-NF), herein incorporated by reference in its entirety.

The parenteral formulation can be any of the five general types of preparations identified by the USP-NF as suitable for parenteral administration:
(1) “Drug Injection:” a liquid preparation that is a drug substance (e.g., a compound of the invention), or a solution thereof;
(2) “Drug for Injection:” the drug substance (e.g., a compound of the invention) as a dry solid that will be combined with the appropriate sterile vehicle for parenteral administration as a drug injection;
(3) “Drug Injectable Emulsion:” a liquid preparation of the drug substance (e.g., a compound of the invention) that is dissolved or dispersed in a suitable emulsion medium;
(4) “Drug Injectable Suspension:” a liquid preparation of the drug substance (e.g., a compound of the invention) suspended in a suitable liquid medium; and
(5) “Drug for Injectable Suspension:” the drug substance (e.g., a compound of the invention) as a dry solid that will be combined with the appropriate sterile vehicle for parenteral administration as a drug injectable suspension.

Exemplary formulations for parenteral administration include solutions of the compound prepared in water suitably mixed with a surfactant, e.g., hydroxypropylcellulose. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington: The Science and Practice of Pharmacy, 23^{rd} Ed., Adejare, Ed., Academic Press (2020) and in The United States Pharmacopeia and National Formulary (USP 43 NF38), published in 2019.

Formulations for parenteral administration may, for example, contain sterile water, saline, polyalkylene glycols (e.g., polyethylene glycol), oils of vegetable origin, or hydrogenated naphthalenes. Biocompatible, biodegradable lactide polymer, lactide/glycolide copolymer, or polyoxyethylene-polyoxypropylene copolymers may be used to control the release of the compounds. Other potentially useful parenteral delivery systems for compounds include ethylene-vinyl acetate copolymer particles, osmotic pumps, implantable infusion systems, and liposomes. Formulations for inhalation may contain, for example, lactose, or may be aqueous solutions containing, for example, polyoxyethylene-9-lauryl ether, glycocholate and deoxycholate, or may be oily solutions for administration in the form of nasal drops, or as a gel.

The parenteral formulation can be formulated for prompt release or for sustained/extended release of the compound. Exemplary formulations for parenteral release of the compound include: aqueous solutions, powders for reconstitution, cosolvent solutions, oil/water emulsions, suspensions, oil-based solutions, liposomes, microspheres, and polymeric gels.

### Methods of Treatment

The compounds of formula (I), e.g., Compounds 1-106, are, in general, suitable for any therapeutic use, e.g., where modulation of FGF activity is desired. In some embodiments, compounds of formula (I), e.g., Compounds 1-106, may be used to treat any disease or disorder that may benefit from increased activity of FGF, for example, stroke, e.g., acute stroke and/or stroke in a recovery phase; congenital hypogonadotropic hypogonadism (e.g., Kallmann Syndrome); cerebral hemorrhage; traumatic brain injury (TBI); spinal cord injury (SCI); peripheral vascular disease (PVD); wounds, i.e., for wound healing; bone or cartilage injury; hearing loss; depression; anxiety; post-traumatic stress disorder (PTSD); substance abuse; peripheral nerve injury; hematopoietic disorders; amyotrophic lateral sclerosis (ALS); Alzheimer's disease; Parkinson's disease; heart disease; non-arteritic ischemic optic neuropathy (NAION); retinal artery occlusion; bronchopulmonary dysplasia, muscular dystrophy, anosmia, aging, memory disturbance, or viral infection.

Increased activity of FGF, e.g., FGF-2, has beneficial effects in cardiovascular, cerebrovascular, and peripheral vascular disease, including enhancement of functional recovery after stroke (Wada et al. Stroke 2003; 34:2724; Kawamata et al. Proc. Natl. Acad. Sci. USA 1997; 94:8179;) and TBI (Dietrich et al. Journal of Neurotrauma 1996; 13:309; McDermott et al. Journal of Neurotrauma 1997; 14:191). In some embodiments, the compounds of formula (I), e.g., Compounds 1-106, may be used to treat or enhance a subject's recovery from brain injuries and diseases, preferably cerebrovascular diseases, e.g., stroke and TBI, and conditions associated therewith (e.g., anosmia associated with TBI).

In particular, the compounds, pharmaceutical compositions, and methods of the invention may be used to enhance the recovery of subjects who had suffered a brain injury or disease, e.g., stroke or TBI. In some embodiments, the stroke may be an acute stroke. In some embodiments, the stroke may be an acute ischemic stroke. In some embodiments, the compounds of formula (I), e.g., Compounds 1-106, may be used to treat acute stroke by administering the compounds of formula (I), e.g., Compounds 1-106, to a stroke subject within the first day after the stroke. In other embodiments, the compounds of formula (I), e.g., Compounds 1-106, may be used to treat and/or enhance functional recovery after stroke, i.e., stroke in a recovery phase, by administering the compounds of formula (I), e.g., Compounds 1-106, to a stroke subject more than one day (e.g., days to years) after the stroke.

FGF may be used in the treatment of neurological diseases because of its neuroprotective properties and effects on neuronal proliferation (see, e.g., Katsouri et al. Neurobiol. Aging. 2015; 36(2): 821-31; Kiyota et al. Proc. Natl. Acad. Sci. 2011; 108(49): E1339-48; Ma et al. Curr. Pharm. Des. 2007; 13(15): 1607-16; and Woodbury et al. J. Neuroimmune Pharmacol. 2014; 9(2): 92-101). In some embodiments, the compounds of formula (I), e.g., Compounds 1-106, may be used to treat or enhance recovery from neurological diseases, e.g., Alzheimer's disease, Parkinson's disease, and ALS. In yet other embodiments, the compounds of formula (I), e.g., Compounds 1-106, may be used to treat or enhance recovery from diseases, disorders, or medical symptoms related to memory disturbance.

FGF has been shown to be neuroprotective and therapeutic for hearing loss (see, e.g., D'Sa et al. Eur J Neurosci. 2007; 26:666-80; Zhang et al. Lin Chuang Er Bi Yan Hou Ke Za Zhi. 2002; 16:603-4; Zhai et al. Acta Otolaryngol. 2004; 124:124-9; Wimmer et al. Otol Neurotol. 2004; 25:33-40; Sekiya et al. Neurosurgery. 2003; 52:900-7; Smith et al. Hear Res. 2002; 169:1-12; Zhai et al. Zhonghua Er Bi Yan Hou Ke Za Zhi. 199; 32:354-6). Accordingly, the compounds of formula (I), e.g., Compounds 21-106, may be used to treat or prevent hearing loss.

FGF has been shown to modulate affective and addictive disorders (Turner et al. Neuron 2012; 76:160; Turner et al. Brain Res. 2008; 1224:63-68). In some preferred embodiments, the compounds of formula (I), e.g., Compounds 1-106, may be used to treat or enhance recovery from diseases, disorders, or medical symptoms related to PTSD, anxiety, or depression. In other preferred embodiments, the compounds of formula (I), e.g., Compounds 1-106, may be used to treat or enhance recovery from diseases, disorders, or medical symptoms related to substance abuse.

FGF has been shown to induce proliferation of progenitor and stem cells (Wada et al. Stroke 2003; 34:2724) and enhance axon regeneration (Haenzi et al. Neural Plasticity. 2017: 2740768). In some embodiments, the compounds of formula (I), e.g., Compounds 1-106, may be used to induce stem cell proliferation and differentiation, e.g., in the brain. The compounds of formula (I), e.g., Compounds 1-106, may also be used to induce stem cell proliferation and differentiation, preferably stem cell proliferation and differentiation in the brain. Similarly, in some embodiments, the compounds of formula (I), e.g., Compounds 1-106, may be used to treat or enhance recovery from peripheral nerve injury or lesion and heart disease. In some embodiments, the compounds of formula (I), e.g., Compounds 1-106, may be used to treat or enhance recovery from cerebral hemorrhage or spinal cord injury.

FGF has been shown to induce bone and cartilage formation and repair (Aspenberg et al. Acta Orthop Scand. 1989; 60:473-6; Chuma et al. Osteoarthritis Cartilage. 2004; 12:834-42). In some embodiments, the compounds of formula (I), e.g., Compounds 1-106, may be used to treat or enhance recovery from diseases and disorders related to bone and cartilage formation or to aid bone and cartilage formation. In some embodiments, the compounds of formula (I), e.g., Compounds 1-106, may be used to induce wound healing.

FGF-2 has been shown to promote in vivo muscle regeneration in murine muscular dystrophy (Lefaucheur et al. Neuroscience Letters. 1995; 202: 121-124). In some embodiments, the compounds of formula (I), e.g., Compounds 1-106, may be used to treat muscular dystrophy in a subject.

FGF has also been shown to promote hematopoiesis (Zhao et al. Blood. 2012; 120:1831). In some embodiments, the compounds of formula (I), e.g., Compounds 1-106, may be used to induce hematopoiesis. Hematopoiesis includes, but is not limited to, hematopoiesis in the brain and the bone marrow. The compounds of formula (I), e.g., Compounds 1-106, may also be used to induce hematopoiesis, e.g., hematopoiesis in the brain and the bone marrow.

Mutations in FGFR1 that cause loss or reduction of function have been implicated in several conditions including hypogonadotropic hypogonadism or conditions (e.g., Kallmann syndrome, anosmia, and normosmic idiopathic hypogonadotropic hypogonadism; see, e.g., Valdes-Socin et al. Front. Endocrinol. 2014; 5: 109 and Miraoui et al., Mol. Cell. Endocrinol. 2011; 346(1-2): 37-43). Such mutations result in reduced tyrosine kinase activity, cell surface expression, and/or reduced affinity for FGF (Pitteloud et al. Proc. Natl. Acad. Sci. USA 2006; 103:6281-67286; Raivio et al. J Clin. Endocrinol. Metab. 2009, 94:4380-4390). Increasing signaling via FGFR1 may therefore treat hypogonadotropic hypogonadism (e.g., Kallmann syndrome, and normosmic idiopathic hypogonadotropic hypogonadism) and conditions associated therewith (e.g., anosmia). The compounds of formula (I), e.g., Compounds 1-106, may also be used to increase signaling activity of FGFR1 and enhance the binding between FGFR1 and its ligands, thereby treating hypogonadotropic hypogonadism (e.g., Kallmann syndrome, and normosmic idiopathic hypogonadotropic hypogonadism) and conditions associated therewith (e.g., anosmia).

FGF affords protective effects on ischemia induced retinal injury (Unoki et al. Invest Ophthalomol. Vis. Sci. 1994; 35:907-915). In some embodiments, the compounds of formula (I), e.g., Compounds 1-106, may be used to treat or enhance recovery from an ocular arterial occlusive disorder, e.g., non-arteritic anterior ischemic optic neuropathy (NAION) or retinal artery occlusion.

The impairment of alveolar formation is the prominent feature of bronchopulmonary dysplasia, and FGF signaling is critical for alveologenesis (Bourbon et al., Pediatr. Res. 2005; 57: 38-46). In some embodiments, the compounds of formula (I), e.g., Compounds 1-106, may also be used to enhance FGF signaling, thereby treating bronchopulmonary dysplasia.

The aging process has been associated with cellular senescence and a decline in somatic stem cell numbers and self-renewal within multiple tissues (Coutu et al. Aging. 2011; 3:920-933). FGFs and FGFRs are key regulators of both senescence and self-renewal in a variety of stem cell types. In some embodiments, the compounds of formula (I), e.g., Compounds 1-106, may be used to modulate FGF signaling, thereby counteracting the effects of aging.

FGF has been shown to be crucial for the development of the vertebrate olfactory epithelium (OE) and the maintenance of OE neurogenesis during prenatal development (Kawauchi et al. Development. 2006; 132(23): 5211-23) and has also been shown to effect recovery of neural anosmia in mice by facilitating olfactory neuron regeneration (Nota et al. JAMA Otolaryngol. Head Neck Surg. 2013; 139: 398). In some embodiments, the compounds of formula (I), e.g., Compounds 1-106, may be used for treating anosmia (e.g., anosmia associated with impaired olfactory neuron development or regeneration, olfactory neuron degeneration, or death of olfactory neurons).

FGF has been shown to inhibit viral replication (van Asten et al. J. Virol. 2018; 92: e00260-18). In some embodiments, the compounds of formula (I), e.g., Compounds 1-106, may be used to treat a viral infection.

FGF signaling has been shown to increase spermatogenesis (Cotton et al. J. Cell. Sci. 20016; 119: 75-84; Saucedo et al. J Cell Physiol. 2018; 233(12): 9640-9651. In some embodiments, the compounds of formula (I), e.g., Compounds 1-106, may be used to increase spermatogenesis in a subject.

The dosage of the pharmaceutical compositions of the invention depends on factors including the route of administration, the disease to be treated, and physical characteristics, e.g., age, weight, and general health, of the subject. Typically, the amount of a compound of formula (I), e.g., any one of Compounds 1-106, contained within a single dose may be an amount that effectively treats the disease without inducing significant toxicity. A pharmaceutical composition of the invention may include a dosage of a compound of formula (I), e.g., any one of Compounds 1-106, ranging from 0.001 to 500 mg/kg/day and, in a more specific embodiment, about 0.1 to about 100 mg/kg/day and, in a more specific embodiment, about 0.3 to about 30 mg/kg/day. The dosage may be adapted by the clinician in accordance with conventional factors such as the extent of the disease and different parameters of the subject. Typically, a pharmaceutical composition of the invention can be administered in an amount from about 0.001 mg up to about 500 mg/kg/day (e.g., 0.05, 0.01, 0.1, 0.2, 0.3, 0.5, 0.7, 0.8, 1 mg, 2 mg, 3 mg, 4 mg, 5 mg, 10 mg, 15 mg, 20 mg, 30 mg, 50 mg, 100 mg, 250 mg, or 500 mg) of a compound of formula (I), e.g., any one of Compounds 1-106.

Pharmaceutical compositions of the invention that contain a compound of formula (I), e.g., any one of Compounds 1-106, may be administered to a subject in need thereof, e.g., subjects who had suffered a brain injury or disease, e.g., a stroke or TBI, one or more times (e.g., 1-10 times or more) daily, weekly, monthly, biannually, annually, or as medically necessary. Preferably, the compounds of formula (I), e.g., Compounds 1-106, may be administered on at least two consecutive days, e.g., on at least 3 consecutive days. Dosing on multiple days may be particularly beneficial in stroke recovery. Preferably, a subject may be administered a therapeutically effective amount of a compound of formula (I), e.g., any one of Compounds 1-106, or a pharmaceutical composition of the invention within the first month (e.g., within 30, 25, 20, 15, 10, 5, or 1 day) after onset of disease or injury, e.g., stroke or TBI. The timing between administrations may decrease as the medical condition improves or increase as the health of the subject declines.

### EXAMPLES

### Example 1. Compound Preparation

The general procedures used to synthesize the compounds are described in reaction Schemes 1-14 and are illustrated in the examples below. The following examples are put forth so as to provide those of ordinary skill in the art with a complete disclosure and description of how to make and use the present invention and are not intended to limit the scope of the invention, nor are they intended to represent that the experiments below were performed or that they are all of the experiments that may be performed. It is to be understood that exemplary description written in the present tense were not necessarily performed, but rather that the descriptions can be performed to generate data and the like of a nature described therein. Synthesized compounds were analyzed and characterized by use of the following equipment: Liquid chromatography-mass spectra (LC/MS) were obtained using an Agilent LC/MSD G1946D or an Agilent 1100 Series LC/MSD Trap G1311A or G2435A. Quantifications were obtained on a Cary 50 Bio UV-visible spectrophotometer. ¹H, ¹³C, and ¹⁹F nuclear magnetic resonance (NMR) spectra were obtained using a Varian INOVA NMR spectrometer at 400, 100, and 376 MHz, respectively. High-performance liquid chromatography (HPLC) analytical separations were performed on an Agilent 1100 or Agilent 1200 HPLC analytical system and followed by an Agilent Technologies G1315B Diode Array Detector set at or near the UVmax @ 210 nm. HPLC preparatory separations were performed on a Gilson preparative HPLC system or an Agilent 1100 preparative HPLC system and followed by an Agilent Technologies G1315B Diode Array Detector set at or near the UVmax @ 210 nm. Analytical chiral HPLC separations were performed on an Agilent 1100 analytical system and followed by an Agilent Technologies G1315B Diode Array Detector set at or near the UVmax @ 210 nm. The separations were accomplished with a Gemini 3 μm or 5 μm C_{18 50×2.5} mm or 250×4.6 mm solid-phase column eluting with acetic acid-methanol-water gradient or ammonium acetate-acetonitrile-water gradient. Flash chromatography was performed using CombiFlash NextGen 300+ using RediSep Silica columns. All final compounds gave satisfactory purity (>95%) by HPLC and by ¹H NMR spectroscopy. Thin-layer chromatography (TLC) analyses are performed on Uniplate 250 μm silica gel plates (Analtech, Inc. Catalog no. 02521) and were typically developed for visualization by UV/Vis, using 50 vol % concentrated sulfuric acid in water spray, iodine stain, or Hanessian's stain.

### Abbreviations

In describing the invention, chemical elements are identified in accordance with the Periodic Table of Elements. Abbreviations and symbols utilized herein are in accordance with the common usage of such abbreviations and symbols by those skilled in the chemical arts. The following abbreviations are used herein:
ACN acetonitrile
AcOEt ethyl acetate
AcOH acetic acid
APCI atmospheric pressure chemical ionization
Boc tert-butyloxycarbonyl
DCE 1,2-dichloroethane
DCM dichloromethane
DIPEA diisopropylamine
DMAP 4-dimethylamino pyridine
DMSO-d6 deuterated dimethylsulfoxide
DMSO dimethylsulfoxide
EtOH ethanol
Et₂NH diethylamine
g gram(s)
Hep heptane
Hex hexane
h hours
H₂O water
HOAC acetic acid
HPLC high pressure liquid chromatography
I₂ Iodine
i-PrOH isopropanol
MeOH methanol
MgSO₄ magnesium sulfate
min minutes
mg milligram(s)
mmol millimolar
mol mole
N₂ Nitrogen
NaCl sodium chloride
NaHCO₃ sodium bicarbonate
Na₂SO₄ sodium sulfate
NaO^{t}Bu sodium tert-butoxide
NaBH(OAc)₃ sodium triacetoxyborohydride
NMR Nuclear Magnetic Resonance spectroscopy
Pd₂(dba)₃ Tris(dibenzylideneacetone)dipalladium (0)
R_{f} retention factor
50 RT room temperature
Rt retention time
RuPhos 2-Dicyclohexylphosphino-2′,6′-diisopropoxybiphenyl
TEA triethylamine
TFA trifluoracetic acid
THE tetrahydrofuran

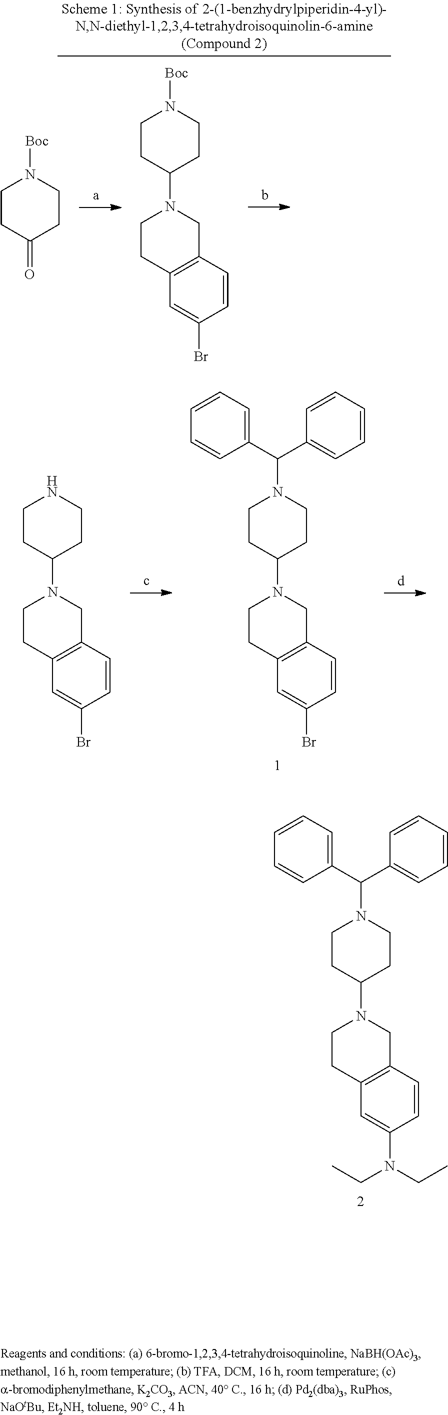

### Step a: Preparation of tert-butyl 4-(6-bromo-3,4-dihydroisoquinolin-2(1H)-yl)piperidine-1-carboxylate

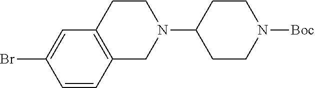 [SMILES: CC(C)(C)OC(=O)N1CCC(N2CCc3cc(Br)ccc3C2)CC1]

To a mixture of 1-Boc-4-piperidinone (Chem Impex, 2.82 g, 14.1 mmol) and 6-bromo-1,2,3,4-tetrahydroisoquinoline (Matrix Scientific, 2.50 g, 11.8 mmol) in methanol (30 mL) was added sodium triacetoxyborohydride (Arctom Chemicals, 6.20 g, 29.5 mmol). The reaction mixture was stirred overnight at room temperature under N₂ atmosphere. The reaction mixture was subsequently partitioned between ethyl acetate (200 mL) and H₂O (200 mL). The phases were separated, and the organic phase was partitioned a second time with H₂O (100 mL), followed by brine (150 mL). The organic layer was separated and dried over anhydrous sodium sulfate, filtered, and concentrated under reduced pressure to give a crude yellow solid. The yellow solid was triturated with ethyl acetate, filtered and set aside. The filtrate was purified by flash silica column chromatography on a CombiFlash NextGen 300+ purification system. Elution through a 40 g RediSep Gold Rf flash silica cartridge with 10-100% ethyl acetate in hexanes afforded the title compound as a yellow oil (2.16 g, 46% yield); Rf 0.18 with 1:1 v/v hexanes-ethyl acetate (12 stain); ¹H-NMR (400 MHz; CDCl₃) δ 7.23 (m, 2H), 6.89 (d, 1H, J=7.8 Hz), 4.19 (bs, 2H), 3.77 (s, 2H), 2.89 (m, 4H), 2.76-2.70 (m, 4H), 1.8-1.9 (m, 1H), 1.82 (m, 1H), 1.82 (m, 1H), 1.45 (s, 9H); MS (APCI⁺) m/z 396.15 (M+1).

### Step b: Preparation of 6-bromo-2-(piperidin-4-yl)-1,2,3,4-tetrahydroisoquinoline

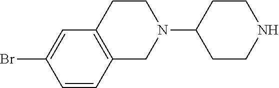 [SMILES: Brc1ccc2c(c1)CCN(C1CCNCC1)C2]

To a solution of tert-butyl 4-(6-bromo-3,4-dihydroisoquinolin-2(1H)-yl)piperidine-1-carboxylate (1.94 g, 4.91 mmol) in dichloromethane (50 mL) was added TFA (5.59 g, 49.1 mmol). The reaction mixture was stirred overnight at room temperature under N₂ atmosphere. The mixture was subsequently concentrated under reduced pressure and chased with toluene (2×100 mL). The crude residue was purified by purified by flash silica column chromatography on a CombiFlash NextGen 300+ purification system. Elution through a 40 g RediSep Gold Rf flash silica cartridge with 20% MeOH in DCM afforded the title compound as a white solid (1.67 g, quant. yield); Rf 0.05 with 1:1 v/v hexanes-ethyl acetate (1₂ stain); MS (APCI⁺) m/z 295.10, 297.10 (M+1).

### Step c: Preparation of 2-(1-benzhydrylpiperidin-4-yl)-6-bromo-1,2,3,4-tetrahydroisoquinoline (Compound 1)

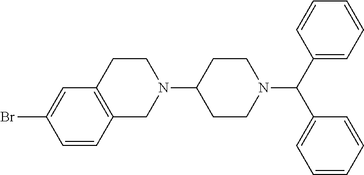 [SMILES: Brc1ccc2c(c1)CCN(C1CCN(C(c3ccccc3)c3ccccc3)CC1)C2]

To a solution of 6-bromo-2-(piperidin-4-yl)-1,2,3,4-tetrahydroisoquinoline (1.44 g, 4.91 mmol) in ACN (50 mL) was added K₂CO₃ (VWR, 1.42 g, 10.3 mmol), followed by addition of α-bromodiphenylmethane (TCl, 1.27 g, 5.16 mmol). The reaction mixture was stirred overnight at 40° C. under N₂ atmosphere. Once the reaction had reached completion it was cooled to room temperature. Next, a white solid, which remained in the crude reaction mixture, was filtered over a fritted funnel and rinsed with excess ACN (100 mL) and ethyl acetate (100 mL). The filtered solid was set aside and the filtrate was partitioned with H₂O (150 mL). The phases were separated, and the organic phase was partitioned with brine (100 mL). The organic layer was separated and concentrated under reduced pressure to afford the crude product. The crude solid was recrystallized in ethyl acetate/heptane (30/70 mixture, 30 mL) to afford the title compound as a white solid (0.940 g, 41%); R_{f} 0.73 with 1:1 v/v hexanes-ethyl acetate (12 stain); ¹H-NMR (400 MHz; CDCl₃) δ 7.39 (br d, 4H, J=7.4 Hz), 7.26 (br t, 6H, J=7.4 Hz), 7.1-7.2 (m, 2H), 6.99 (br d, 1H, J=8.2 Hz), 4.26 (s, 1H), 3.29 (s, 1H), 2.82 (br d, 2H, J=10.5 Hz), 2.60-2.80 (m, 4H), 2.2-2.40 (m, 2H), 1.82 (br t, 2H, J=11.3 Hz), 1.72 (br d, 2H, J=10.9 Hz), 1.52-1.65 (m, 2H); MS (APCI⁺) m/z 461.1, 463.1 (M+1). HPLC UV purity, Rt=12.495 min, 95.18%.

### Step d: Preparation of 2-(1-benzhydrylpiperidin-4-yl)-N,N-diethyl-1,2,3,4-tetrahydroisoquinolin-6-amine (Compound 2)

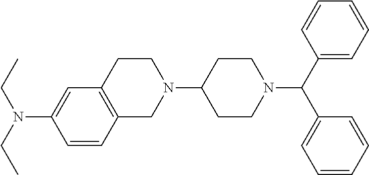 [SMILES: CCN(CC)c1ccc2c(c1)CCN(C1CCN(C(c3ccccc3)c3ccccc3)CC1)C2]

To a solution of Compound 1 (0.940 g, 2.0 mmol) in anhydrous toluene (50 mL) was added a pre-dissolved solution containing Pd₂(dba)₃ (Strem, 0.185 g, 0.203 mmol), and RuPhos (CombiBlocks, 0.189 g, 0.406 mmol) in toluene (10 mL). Next, diethylamine (Alfa Aesar, 3 mL, 30.5 mmol) was added, followed by the addition of NaO^{t}Bu (AK Scientific, 0.292 g, 3.05 mmol). The reaction mixture was stirred overnight at 90° C. under N₂ atmosphere and was subsequently partitioned between ethyl acetate (200 mL) and H₂O (200 mL). The phases were separated, and the organic phase was partitioned a second time with H₂O (150 mL), followed by brine (150 mL). The organic layer was separated and dried over anhydrous sodium sulfate, filtered and concentrated under reduced pressure to give a crude orange solid.

The crude solid was purified by flash silica column chromatography on a CombiFlash NextGen 300+ purification system. Elution through a 40 g RediSep Gold Rf flash silica cartridge with 5-100% ethyl acetate in hexanes afforded the title Compound as a yellow oil that solidifies upon standing (0.195 g, 21% yield); Rf 0.35 with 1:1 v/v hexanes-ethyl acetate (12 stain); ¹H-NMR (400 MHz; CDCl₃) δ 7.39 (br d, 4H, J=7.4 Hz), 7.26 (br t, 4H, J=7.4 Hz), 7.1-7.2 (m, 2H), 6.78 (br d, 1H, J=8.6 Hz), 6.43 (br d, 1H, J=8.6 Hz), 6.32 (br s, 1H), 4.26 (s, 1H), 3.30 (s, 1H), 3.2-3.3 (m, 4H), 2.81 (br d, 2H, J=10.9 Hz), 2.65 (s, 4H), 2.30 (m, 1H), 1.7-1.9 (m, 4H), 1.4-1.6 (m, 2H), 1.01 (br t, 6H, J=6.8 Hz); MS (APCI⁺) m/z 454.30 (M+1); HPLC UV purity, Rt=10.450 min, 94.29%; melting point=90.2-92.7° C.

### Scheme 2: General Method for the Synthesis of 2-(1-Benzhydrylpiperidin-4-yl)-1,2,3,4-Tetrahydroisoquinoline Derivatives (Compounds 3 and 5-20) and 2-(1-Benzhydrylpiperidin-4-yl)1,2,3,4-tetrahydro-2,7-naphthyridine (Compound 4)

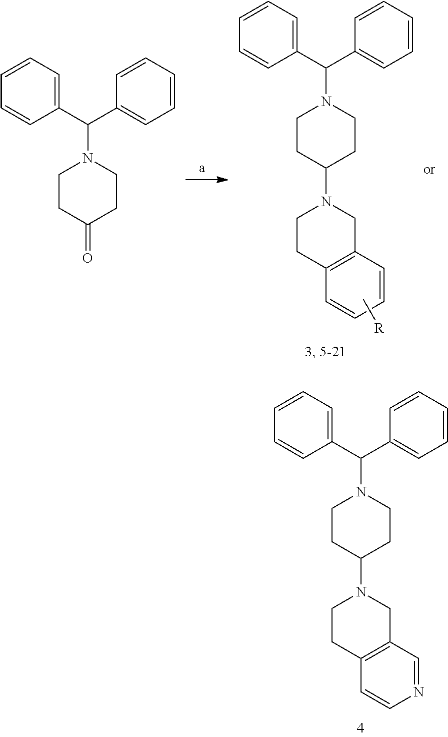

Reagents and conditions: (a) a-s, NaBH(OAc)₃, (THF/methanol/1,2-DCE), (TEA/acetic ccid/both), 4-72 h, rt to 75° C. a=6-carbonitrile-1,2,3,4-tetrahydroisoquinoline; b=1,2,3,4,-tetrahydro-2,7-naphthyridine hydrochloride; c=6-chloro-1,2,3,4-tetrahydroisoquinoline; d=6-fluoro-1,2,3,4,-tetrahydroisoquinoline; e=5-fluoro-1,2,3,4-tetrahydroisoquinoline; f=7-fluoro-1,2,3,4-tetrahydroisoquinoline; g=8-fluoro-1,2,3,4-tetraydroisoquinoline; h=6-(trifluoromethoxy)-1,2,3,4-tetrahydroisoquinoline hydrochloride; i=5-fluoro-1,2,3,4-tetrahydro-2,7-naphthyridine; j=6-(trifluoromethyl)-1,2,3,4-tetrahydro-2,7-naphthyridine; k=5,7-difluoro-1,2,3,4-tetrahydroisoquinoline; I=5,6,7,8-tetrahydro-1,7-naphthyridine; m=4-bromo-5,6,7,8-tetrahydro-1,7-naphthyridine; n=3-fluoro-5,6,7,8-tetrahydro-1,7-naphthyridine; o=5,6,7,8-Tetrahydro-1,6-naphthyridine; p=3-fluoro-5,6,7,8-tetrahydro-1,6-naphthyridine; q=1,2,3,4-tetrahydro-2,6-naphthyridine; r=7-(trifluoromethyl)-1,2,3,4-tetrahydro-2,6-naphthyridine; s=4,4-difluoro-1,2,3,4-tetrahydroisoquinoline.

### Preparation of 2-(1-benzhydrylpiperidin-4-yl)-1,2,3,4-tetrahydroisoquinoline-6-carbonitrile (Compound 3)

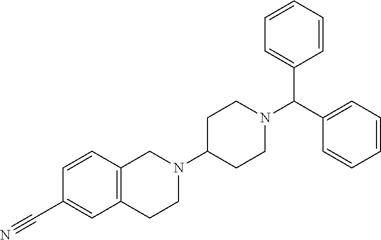 [SMILES: N#Cc1ccc2c(c1)CCN(C1CCN(C(c3ccccc3)c3ccccc3)CC1)C2]

To a mixture of 1-benzhydrylpiperidin-4-one (BLDpharm, 0.300 g, 0.99 mmol) and 6-carbonitrile-1,2,3,4-tetrahydroisoquinoline (AmBeed, 0.231 g, 1.19 mmol) in THE (20 mL) was added sodium triacetoxyborohydride (Arctom Chemicals, 0.315 g, 7.85 mmol). The reaction mixture was stirred overnight at 35° C. under N₂ atmosphere. Once the reaction reached completion, the reaction mixture was partitioned between ethyl acetate (200 mL) and H₂O (200 mL). The phases were separated, and the organic phase was partitioned a second time with H₂O (100 mL), followed by brine (150 mL). The organic layer was separated and dried over anhydrous sodium sulfate, filtered, and concentrated under reduced pressure to give the crude product. The crude product was purified by flash silica column chromatography on a CombiFlash NextGen 300+ purification system. Elution through a 120 g RediSep Gold Rf flash silica cartridge with 0-50% ethyl acetate in hexanes afforded the title compound as an off-white solid (0.023 g, 6% yield); Rf 0.14 with 20:80 v/v hexanes-ethyl acetate (UV); ¹H-NMR (400 MHz; CDCl₃) δ 7.38-7.42 (m, 4H), 7.27-7.30 (m, 5H), 7.19-7.21 (m, 2H), 7.12 (d, 1H, J=8.2 Hz), 4.28 (s, 1H), 3.83 (s, 2 h), 3.68 (s, 1H), 3.00 (br d, 2H, J=10.9 Hz), 2.8-2.9 (m, 4H), 2.4-2.5 (m, 1H), 1.81-1.9 (m, 5H), 1.50-1.74 (m, 3H); MS (APCI⁺) m/z 408.20 (M+1); HPLC UV purity, Rt=9.844 min, 97.69%; melting point=132-134° C.

### Preparation of 2-(1-benzhydrylpiperidin-4-yl)-1,2,3,4-tetrahydro-2,7-naphthyridine (Compound 4)

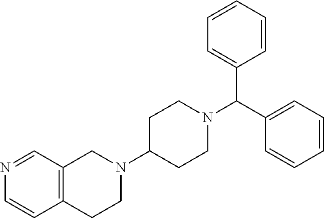 [SMILES: c1ccc(C(c2ccccc2)N2CCC(N3CCc4ccncc4C3)CC2)cc1]

To a mixture of 1-benzhydrylpiperidin-4-one (BLDpharm, 0.301 g, 0.99 mmol) and 1,2,3,4-tetrahydro-2,7-naphthyridine hydrochloride (Combi-Blocks, 0.203 g, 1.19 mmol) in THE (20 mL) was added sodium triacetoxyborohydride (Arctom Chemicals, 0.315 g, 1.49 mmol). The reaction mixture was stirred overnight at 35° C. under N₂ atmosphere. Once the reaction reached completion, the reaction mixture was partitioned between ethyl acetate (200 mL) and H₂O (200 mL). The phases were separated, and the organic phase was partitioned a second time with H₂O (100 mL), followed by brine (150 mL). The organic layer was separated and dried over anhydrous sodium sulfate, filtered, and concentrated under reduced pressure to give the crude product. The crude product was purified by flash silica column chromatography on a CombiFlash NextGen 300+ purification system. Elution through a 4 g RediSep Gold Rf flash silica cartridge with 0-9% methanol in dichloromethane afforded the title compound as an orange oil (0.045 g, 12% yield); Rf 0.50 with 10:90 v/v methanol-dichloromethane (UV); ¹H-NMR (400 MHz; CDCl₃) δ 8.2-8.4 (m, 2H), 7.4-7.5 (m, 4H), 7.2-7.3 (m, 3H), 7.1-7.2 (m, 2H), 7.05 (d, 1H, J=5.1 Hz), 4.31 (s, 1H), 3.81 (s, 2H), 3.03 (br d, 2H, J=9.8 Hz), 2.8-2.9 (m, 4H), 2.5-2.6 (m, 1H), 1.65-1.9 (m, 5H); MS (APCI⁺) m/z 384.24 (M+1); HPLC UV purity, Rt=7.118 min, 89.53%; melting point=110-112° C.

### Alternate Preparation of Compound 4

To a mixture of 1-benzhydrylpiperidin-4-one (1.26 g, 4.7 mmol) and 1,2,3,4-tetrahydro-2,7-naphthyridine hydrochloride (Combi-Blocks, 1.0 g, 5.8 mmol) in 1,2-dichloroethane (25 mL) was added triethylamine (0.5 mL). The reaction mixture was stirred at room temperature for 30 minutes. Acetic acid (0.5 mL) was then added and the solution was stirred for another 30 minutes. Next, sodium triacetoxyborohydride (Arctom Chemicals, 0.890 g, 7.05 mmol) was added and the reaction mixture was stirred overnight at room temperature under N₂ atmosphere. The reaction mixture was quenched with addition of 40 mL of saturated sodium bicarbonate solution. The reaction mixture was portioned in a separatory funnel and the organic layer was extracted with dichloromethane (40 mL) and ethyl acetate (3×30 mL). The combined organic layers were dried over anhydrous sodium sulfate, filtered, and concentrated under reduced pressure to give the crude product. The crude product was purified by flash silica column chromatography on a CombiFlash NextGen 300+ purification system. Elution through a 40 g RediSep Gold Rf flash silica cartridge with 20-100% ethyl acetate in heptane/TEA (0.5%) mixture afforded the title compound as an off-white foamy solid (0.910 g, 51% yield); R_{f} 0.50 with 10:90 v/v methanol-dichloromethane (UV); ¹H-NMR (400 MHz; DMSO-d₆) δ 8.23 (s, 1H), 8.20 (d, 1H, J=5.0 Hz) 7.38 (d, 4H, J=7.8 Hz), 7.25 (t, 4H, J=7.6 Hz), 7.1-7.2 (m, 2H), 7.05 (d, 1H, J=5.0 Hz), 4.25 (s, 1H), 3.81 (s, 2H), 2.81 (br d, 2H, J=11.0 Hz), 2.71 (br s, 4H), 2.3-2.4 (m, 1H), 1.81 (br t, 2H, J=11.2 Hz), 1.71 (br d, 2H, J=11.5 Hz), 1.5-1.6 (m, 2H); MS (APCI⁺) m/z 384.20 (M+1); HPLC UV purity, Rt=8.383 min, 98.2%; melting point=110-112° C.

### Preparation of 2-(1-benzhydrylpiperidin-4-yl)-6-chloro-1,2,3,4-tetrahydroisoquinoline (Compound 5)

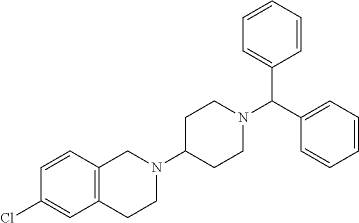 [SMILES: Clc1ccc2c(c1)CCN(C1CCN(C(c3ccccc3)c3ccccc3)CC1)C2]

To a mixture of 1-benzhydrylpiperidin-4-one (BLDpharm, 0.301 g, 0.99 mmol) and 6-chloro-1,2,3,4-tetrahydroisoquinoline (Combi-Blocks, 0.201 g, 1.19 mmol) in THE (20 mL) was added sodium triacetoxyborohydride (Arctom Chemicals, 0.315 g, 1.49 mmol). The reaction mixture was stirred overnight at 35° C. under N₂ atmosphere. Once the reaction reached completion, the reaction mixture was partitioned between ethyl acetate (200 mL) and H₂O (200 mL). The phases were separated, and the organic phase was partitioned a second time with H₂O (100 mL), followed by brine (150 mL). The organic layer was separated and dried over anhydrous sodium sulfate, filtered and concentrated under reduced pressure to give the crude product. The crude product was purified by flash silica column chromatography on a CombiFlash NextGen 300+ purification system. Elution through a 24 g RediSep Gold Rf flash silica cartridge with 0-40% ethyl acetate in hexanes afforded the title compound as an off-white solid (0.061 g, 15% yield); Rf 0.21 with 30:70 v/v hexanes-ethyl acetate (UV); ¹H-NMR (400 MHz; CDCl₃) δ 7.40 (br d, 4H, J=7.4 Hz), 7.2-7.3 (m, 5H), 7.1-7.2 (m, 2H), 7.08 (s, 1H), 7.06 (d, 1H, J=7.4 Hz), 6.94 (d, 1H, J=8.6 Hz), 4.26 (s, 1H), 3.74 (s, 2 h), 2.98 (br d, 2H, J=11.3 Hz), 2.8-2.9 (m, 4H), 2.4-2.5 (m, 1H), 1.65-1.9 (m, 5H); MS (APCI⁺) m/z 417.20 (M+1); HPLC UV purity, Rt=10.653 min, 97.69%; melting point=158.5-160.0° C.

### Preparation of 2-(1-benzhydrylpiperidin-4-yl)-6-fluoro-1,2,3,4-tetrahydroisoquinoline (Compound 6)

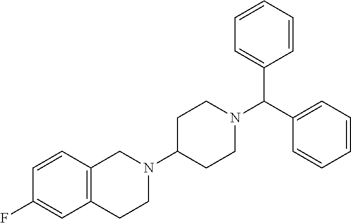 [SMILES: Fc1ccc2c(c1)CCN(C1CCN(C(c3ccccc3)c3ccccc3)CC1)C2]

To a mixture of 1-benzhydrylpiperidin-4-one (BLDpharm, 0.080 g, 0.30 mmol) and 6-fluoro-1,2,3,4-tetrahydroisoquinoline (Enamine, 0.054 g, 0.36 mmol) in THE (1 mL) was added sodium triacetoxyborohydride (Arctom Chemicals, 0.95 g, 0.45 mmol). The reaction mixture was stirred overnight at 35° C. under N₂ atmosphere. Once the reaction reached completion, the reaction mixture was partitioned between ethyl acetate (50 mL) and H₂O (40 mL). The phases were separated, and the organic phase was partitioned a second time with H₂O (40 mL), followed by brine (50 mL). The organic layer was separated and dried over anhydrous sodium sulfate, filtered, and concentrated under reduced pressure to give the crude product. The crude product was purified by flash silica column chromatography on a CombiFlash NextGen 300+ purification system. Elution through a 24 g RediSep Gold Rf flash silica cartridge with 0-50% ethyl acetate in hexanes afforded the title compound as an off-white solid (0.079 g, 66% yield); Rf 0.57 with 50:50 v/v hexanes-ethyl acetate (UV); ¹H-NMR (400 MHz; CDCl₃) δ 7.40 (br d, 4H, J=7.4 Hz), 7.2-7.3 (m, 5H), 7.1-7.2 (m, 2H), 6.96 (dd, 1H, J=5.5, 8.2 Hz), 6.7-6.8 (m, 2H), 4.26 (s, 1H), 3.75 (s, 2 h), 2.98 (br d, 2H, J=11.3 Hz), 2.86 (br dd, 4H, J=4.7, 12.5 Hz), 2.4-2.5 (m, 1H), 1.7-1.9 (m, 5H); ¹⁹F-NMR (400 MHz; CDCl₃) −117.8 ppm; MS (APCI⁺) m/z 401.20 (M+1); HPLC UV purity, Rt=10.103 min, 94.99%; melting point=119.0-121.0° C.

### Preparation of 2-(1-benzhydrylpiperidin-4-yl)-5-fluoro-1,2,3,4-tetrahydroisoquinoline (Compound 7)

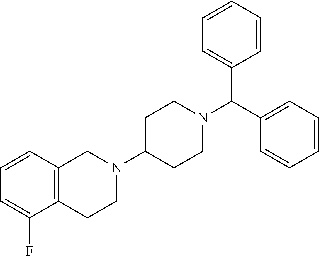 [SMILES: Fc1cccc2c1CCN(C1CCN(C(c3ccccc3)c3ccccc3)CC1)C2]

To a mixture of 1-benzhydrylpiperidin-4-one (BLDpharm, 0.081 g, 0.305 mmol) and 5-fluoro-1,2,3,4-tetrahydroisoquinoline (Enamine, 0.0554 g, 0.366 mmol) in THE (1 mL) was added sodium triacetoxyborohydride (Arctom Chemicals, 0.97 g, 0.45 mmol). The reaction mixture was stirred for two hours at 35° C. under N₂ atmosphere. Once the reaction reached completion, the reaction mixture was partitioned between ethyl acetate (30 mL) and H₂O (40 mL). The phases were separated, and the organic phase was partitioned a second time with H₂O (40 mL), followed by brine (50 mL). The organic layer was separated and dried over anhydrous sodium sulfate, filtered, and concentrated under reduced pressure to give the crude product. The crude product was purified by flash silica column chromatography on a CombiFlash NextGen 300+ purification system. Elution through a 24 g RediSep Gold Rf flash silica cartridge with 0-30% ethyl acetate in hexanes afforded the title compound as an off-white solid (0.071 g, 58% yield); Rf 0.67 with 50:50 v/v hexanes-ethyl acetate (UV); ¹H-NMR (400 MHz; CDCl₃) δ 7.41 (br d, 4H, J=7.4 Hz), 7.2-7.3 (m, 5H), 7.1-7.2 (m, 2H), 7.07 (dt, 1H, J=5.7, 7.9 Hz), 6.8-6.9 (m, 2H), 4.26 (s, 1H), 3.78 (s, 2 h), 2.98 (br d, 2H, J=11.7 Hz), 2.85 (s, 4H), 2.49 (br t, 1H, J=11.1 Hz), 1.8-1.9 (m, 5H); ¹⁹F-NMR (400 MHz; CDCl₃) −119.59 ppm; MS (APCI⁺) m/z 401.20 (M+1); HPLC UV purity, Rt=10.083 min, 94.76%; melting point=114.0-115.4° C.

### Alternate Preparation of Compound 7

To a mixture of 1-benzhydrylpiperidin-4-one (0.533 g, 1.77 mmol) and 5-fluoro-1,2,3,4-tetrahydroisoquinoline (Enamine, 0.418 g, 2.77 mmol) in 1,2-dichloroethane (30 mL) was added triethylamine (1.5 mL). The reaction mixture was stirred at room temperature for 30 minutes. Acetic acid (0.3 mL) was then added and the solution was stirred for another 30 minutes. Next, sodium triacetoxyborohydride (Arctom Chemicals, 0.732 g, 3.45 mmol) was added and the reaction mixture was stirred for 72 hours at room temperature under N₂ atmosphere. The reaction mixture was quenched with 40 mL of saturated sodium bicarbonate solution. The reaction mixture was portioned in a separatory funnel and the organic layer was extracted with dichloromethane (40 mL) and ethyl acetate (3×30 mL). The combined organic layers were dried over anhydrous sodium sulfate, filtered, and concentrated under reduced pressure to give the crude product. The crude product was purified by flash silica column chromatography on a CombiFlash NextGen 300+ purification system. Elution through a 24 g RediSep Rf flash silica cartridge with 0-100% ethyl acetate in heptane mixture afforded the title compound as a white solid (0.560 g, 79% yield); Rf 0.67 with 50:50 v/v hexanes-ethyl acetate (UV); ¹H-NMR (400 MHz; DMSO-d₆) δ 7.42 (d, 4H, J=7.8 Hz), 7.28 (t, 4H, J=7.6 Hz), 7.1-7.2 (m, 2H), 7.12 (df, 1H, J=6.4, 7.8 Hz), 6.9-7.0 (m, 2H), 4.29 (s, 1H), 3.69 (s, 2 h), 2.84 (br d, 2H, J=11.5 Hz), 2.76 (t, 2H, J=5.5 Hz), 2.40 (tt, 1H, J=3.8, 11.1 Hz), 1.84 (br t, 2H, J=11.0 Hz), 1.75 (br d, 2H, J=11.0 Hz), 1.5-1.58 (m, 2H); ¹⁹F-NMR (400 MHz; CDCl₃) −119.59 ppm; MS (APCI⁺) m/z 401.30 (M+1); HPLC UV purity, Rt=10.174 min, 95.64%; melting point=114.0-115.4° C.

### Preparation of 2-(1-benzhydrylpiperidin-4-yl)-7-fluoro-1,2,3,4-tetrahydroisoquinoline (Compound 8)

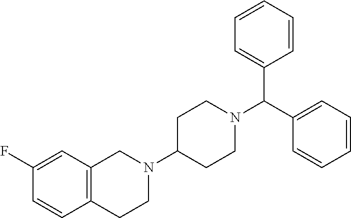 [SMILES: Fc1ccc2c(c1)CN(C1CCN(C(c3ccccc3)c3ccccc3)CC1)CC2]

To a mixture of 1-benzhydrylpiperidin-4-one (0.068 g, 0.26 mmol) and 7-fluoro-1,2,3,4-tetrahydroisoquinoline (Enamine, 0.047 g, 0.31 mmol) in THF (1 mL) was added sodium triacetoxyborohydride (Arctom Chemicals, 0.081 g, 0.38 mmol). The reaction mixture was stirred for two hours at 35° C. under N₂ atmosphere. Once the reaction reached completion, the reaction mixture was partitioned between ethyl acetate (40 mL) and H₂O (40 mL). The phases were separated, and the organic phase was partitioned a second time with H₂O (40 mL), followed by brine (50 mL). The organic layer was separated and dried over anhydrous sodium sulfate, filtered, and concentrated under reduced pressure to give the crude product. The crude product was purified by flash silica column chromatography on a CombiFlash NextGen 300+ purification system. Elution through a 24 g RediSep Gold Rf flash silica cartridge with 0-50% ethyl acetate in hexanes afforded the title compound as an off-white solid (0.061 g, 59% yield); Rf 0.60 with 50:50 v/v hexanes-ethyl acetate (UV); ¹H-NMR (400 MHz; CDCl₃) δ 7.40 (br d, 4H, J=7.0 Hz), 7.2-7.3 (m, 5H), 7.1-7.2 (m, 2H), 7.03 (dd, 1H, J=5.7, 8.4 Hz), 6.82 (dt, 1H, J=2.7, 8.6 Hz), 6.72 (dd, 1H, J=2.7, 9.4 Hz), 4.26 (s, 1H), 3.78 (s, 2 h), 2.98 (br d, 2H, J=11.3 Hz), 2.85 (s, 4H), 2.49 (m, 1H), 1.7-1.9 (m, 5H); ¹⁹F-NMR (400 MHz; CDCl₃) −117.86 ppm; MS (APCI⁺) m/z 401.20 (M+1); HPLC UV purity, Rt=10.136 min, 95.54%; melting point=103.5-105° C.

### Preparation of 2-(1-benzhydrylpiperidin-4-yl)-8-fluoro-1,2,3,4-tetrahydroisoquinoline (Compound 9)

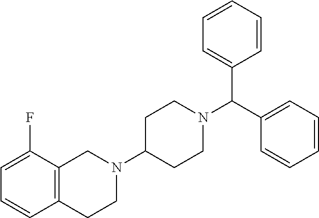 [SMILES: Fc1cccc2c1CN(C1CCN(C(c3ccccc3)c3ccccc3)CC1)CC2]

To a mixture of 1-benzhydrylpiperidin-4-one (BLDpharm, 0.059 g, 0.22 mmol) and 8-fluoro-1,2,3,4-tetrahydroisoquinoline (Enamine, 0.048 g, 0.26 mmol) in THE (20 mL) was added sodium triacetoxyborohydride (Arctom Chemicals, 0.070 g, 0.33 mmol). The reaction mixture was stirred overnight at room temperature under N₂ atmosphere. Once the reaction reached completion, the reaction mixture was partitioned between ethyl acetate (50 mL) and saturated sodium bicarbonate solution (40 mL). The phases were separated, and the organic phase was partitioned a second time with NaHCO₃ (40 mL), followed by brine (50 mL). The organic layer was separated and dried over anhydrous sodium sulfate, filtered, and concentrated under reduced pressure to give the crude product. The crude product was purified by flash silica column chromatography on a CombiFlash NextGen 300+ purification system.

Elution through a 24 g RediSep Gold Rf flash silica cartridge with 0-40% ethyl acetate in hexanes afforded the title compound as a colorless oil (0.022 g, 25% yield); R_{f} 0.15 with 80:20 v/v hexanes-ethyl acetate (UV); MS (APCI⁺) m/z 401.20 (M+1); HPLC UV purity, Rt=10.159 min, 83.82%.

### Preparation of 2-(1-benzhydrylpiperidin-4-yl)-6-(trifluoromethoxy)-1,2,3,4-tetrahydroisoquinoline (Compound 10)

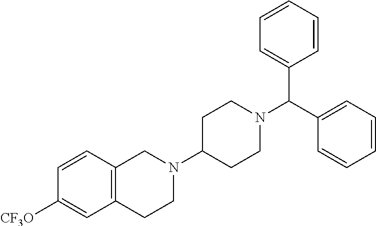

To a mixture of 1-benzhydrylpiperidin-4-one (BLDpharm, 1.2 molar equivalents) and 6-(trifluoromethoxy)-1,2,3,4-tetrahydroisoquinoline hydrochloride (Enamine, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 2-(1-benzhydrylpiperidin-4-yl)-5-fluoro-1,2,3,4-tetrahydro-2,7-naphthyridine (Compound 11)

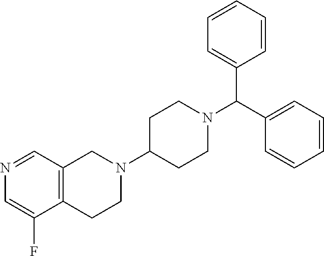 [SMILES: Fc1cncc2c1CCN(C1CCN(C(c3ccccc3)c3ccccc3)CC1)C2]

To a mixture of 1-benzhydrylpiperidin-4-one (BLDpharm, 1.2 molar equivalents) and 5-fluoro-1,2,3,4-tetrahydro-2,7-naphthyridine (Anichem, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 2-(1-benzhydrylpiperidin-4-yl)-6-(trifluoromethyl)-1,2,3,4-tetrahydro-2,7-naphthyridine (Compound 12)

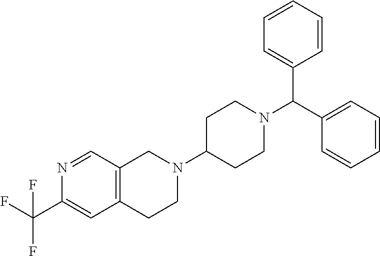 [SMILES: FC(F)(F)c1cc2c(cn1)CN(C1CCN(C(c3ccccc3)c3ccccc3)CC1)CC2]

To a mixture of 1-benzhydrylpiperidin-4-one (BLDpharm, 1.2 molar equivalents) and 6-(trifluoromethyl)-1,2,3,4-tetrahydro-2,7-naphthyridine (Anichem, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with addition of 40 mL of saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 2-(1-benzhydrylpiperidin-4-yl)-5,7-difluoro-1,2,3,4-tetrahydroisoquinoline (Compound 13)

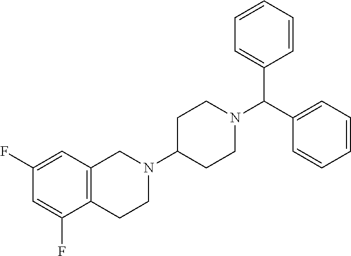 [SMILES: Fc1cc(F)c2c(c1)CN(C1CCN(C(c3ccccc3)c3ccccc3)CC1)CC2]

To a mixture of 1-benzhydrylpiperidin-4-one (BLDpharm, 1.2 molar equivalents) and 5,7-difluoro-1,2,3,4-tetrahydroisoquinoline (Enamine, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 7-(1-benzhydrylpiperidin-4-yl)-5,6,7,8-tetrahydro-1,7-naphthyridine (Compound 14)

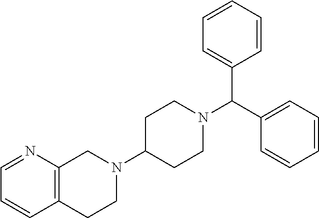 [SMILES: c1ccc(C(c2ccccc2)N2CCC(N3CCc4cccnc4C3)CC2)cc1]

To a mixture of 1-benzhydrylpiperidin-4-one (BLDpharm, 1.2 molar equivalents) and 5,6,7,8-tetrahydro-1,7-naphthyridine (Enamine, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 7-(1-benzhydrylpiperidin-4-yl)-4-bromo-5,6,7,8-tetrahydro-1,7-naphthyridine (Compound 15)

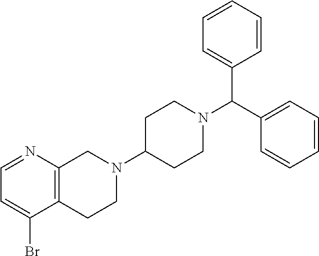 [SMILES: Brc1ccnc2c1CCN(C1CCN(C(c3ccccc3)c3ccccc3)CC1)C2]

To a mixture of 1-benzhydrylpiperidin-4-one (BLDpharm, 1.2 molar equivalents) and 4-bromo-5,6,7,8-tetrahydro-1,7-naphthyridine (Enamine, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 7-(1-benzhydrylpiperidin-4-yl)-3-fluoro-5,6,7,8-tetrahydro-1,7-naphthyridine (Compound 16)

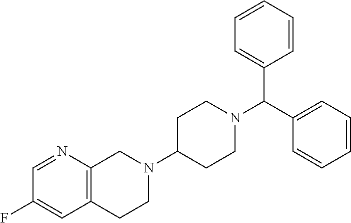 [SMILES: Fc1cnc2c(c1)CCN(C1CCN(C(c3ccccc3)c3ccccc3)CC1)C2]

To a mixture of 1-benzhydrylpiperidin-4-one (BLDpharm, 1.2 molar equivalents) and 3-fluoro-5,6,7,8-tetrahydro-1,7-naphthyridine (Ambeed, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 6-(1-benzhydrylpiperidin-4-yl)-5,6,7,8-tetrahydro-1,6-naphthyridine (Compound 17)

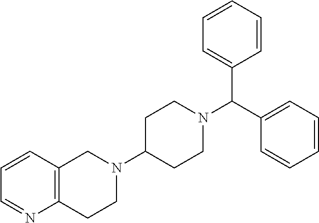 [SMILES: c1ccc(C(c2ccccc2)N2CCC(N3CCc4ncccc4C3)CC2)cc1]

To a mixture of 1-benzhydrylpiperidin-4-one (BLDpharm, 1.2 molar equivalents) and 5,6,7,8-tetrahydro-1,6-naphthyridine (Matrix Scientific, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 6-(1-benzhydrylpiperidin-4-yl)-3-fluoro-5,6,7,8-tetrahydro-1,6-naphthyridine (Compound 18)

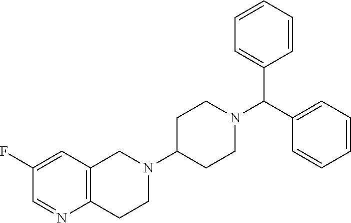 [SMILES: Fc1cnc2c(c1)CN(C1CCN(C(c3ccccc3)c3ccccc3)CC1)CC2]

To a mixture of 1-benzhydrylpiperidin-4-one (BLDpharm, 1.2 molar equivalents) and 3-fluoro-5,6,7,8-tetrahydro-1,6-naphthyridine (Anichem, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 2-(1-benzhydrylpiperidin-4-yl)-1,2,3,4-tetrahydro-2,6-naphthyridine (Compound 19)

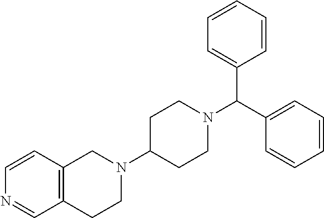 [SMILES: c1ccc(C(c2ccccc2)N2CCC(N3CCc4cnccc4C3)CC2)cc1]

To a mixture of 1-benzhydrylpiperidin-4-one (BLDpharm, 1.2 molar equivalents) and 1,2,3,4-tetrahydro-2,6-naphthyridine (Aldrich, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 2-(1-benzhydrylpiperidin-4-yl)-7-(trifluoromethyl)-1,2,3,4-tetrahydro-2,6-naphthyridine (Compound 20)

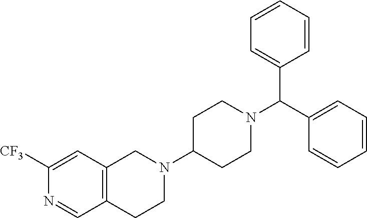 [SMILES: FC(F)(F)c1cc2c(cn1)CCN(C1CCN(C(c3ccccc3)c3ccccc3)CC1)C2]

To a mixture of 1-benzhydrylpiperidin-4-one (BLDpharm, 1.2 molar equivalents) and 7-(trifluoromethyl)-1,2,3,4-tetrahydro-2,6-naphthyridine (Matrix Scientific, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 2-(1-benzhydrylpiperidin-4-yl)-4,4-difluoro-1,2,3,4-tetrahydroisoquinoline (Compound 21)

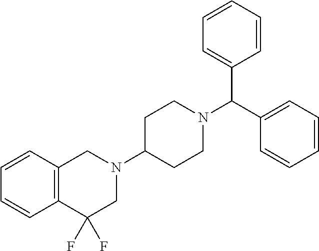 [SMILES: FC1(F)CN(C2CCN(C(c3ccccc3)c3ccccc3)CC2)Cc2ccccc21]

To a mixture of 1-benzhydrylpiperidin-4-one (BLDpharm, 1.2 molar equivalents) and 4,4-difluoro-1,2,3,4-tetrahydroisoquinoline (Enamine, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Scheme 3: General Method for the Synthesis of 2-(1-Benzhydrylpiperidin-4-yl)-1,2,3,4-tetrahydroisoquinolin-6-amine Derivatives (Compounds 22-33) from 2-(1-Benzhydrylpiperidin-4-yl)-6-bromo-1,2,3,4-tetrahydroisoquinoline (Compound 3)

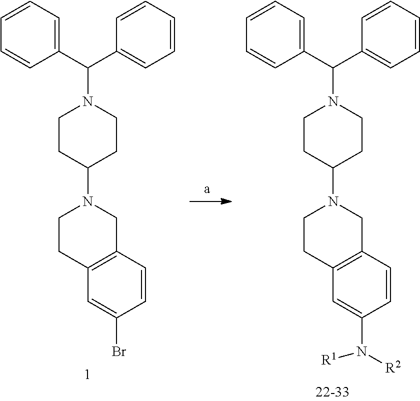

Reagents and conditions: (a) Pd₂(dba)₃, a-l, RuPhos, NaO^{t}Bu, toluene, 90° C., 16 h. a=dimethylamine; b=morpholine; c=piperidine; d=di-n-propylamine; e=diisopropylamine; f=methylamine; g=ethylamine; h=isopropylamine; i=1-propylamine; j=isobutylamine; k=diisobutylamine; I=pyrrolidine.

### Preparation of 2-(1-benzhydrylpiperidin-4-yl)-N,N-dimethyl-1,2,3,4-tetrahydroisoquinolin-6-amine (Compound 22)

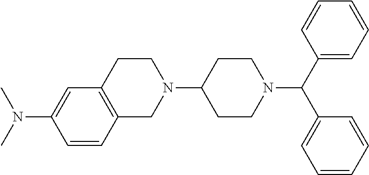 [SMILES: CN(C)c1ccc2c(c1)CCN(C1CCN(C(c3ccccc3)c3ccccc3)CC1)C2]

To a solution of Compound 1 (1.0 molar equivalents) in anhydrous toluene is added a pre-dissolved solution containing Pd₂(dba)₃ (Strem, 0.10 molar equivalents), and RuPhos (CombiBlocks, 0.20 molar equivalents) in toluene. Next, dimethylamine (Alfa Aesar, 15 molar equivalents) is added, followed by NaO^{t}Bu (AK Scientific, 1.5 molar equivalents). The reaction mixture is stirred overnight at 90° C. under N₂ atmosphere. nOnce the reaction reaches completion it is cooled to RT and the reaction mixture is partitioned between ethyl acetate and H₂O. The phases are separated, and the organic phase is partitioned a second time with H₂O, followed by brine. The organic layer is separated and dried over anhydrous sodium sulfate, filtered and concentrated under reduced pressure to afford the crude product. The crude product is purified by flash silica column chromatography to afford the title compound.

### Preparation of 4-(2-(1-benzhydrylpiperidin-4-yl)-1,2,3,4-tetrahydroisoquinolin-6-yl)morpholine (Compound 23)

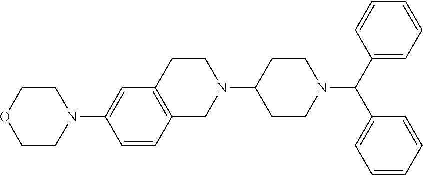 [SMILES: c1ccc(C(c2ccccc2)N2CCC(N3CCc4cc(N5CCOCC5)ccc4C3)CC2)cc1]

To a solution of Compound 1 (1.0 molar equivalents) in anhydrous toluene is added a pre-dissolved solution containing Pd₂(dba)₃ (Strem, 0.10 molar equivalents), and RuPhos (CombiBlocks, 0.20 molar equivalents) in toluene. Next, morpholine (Alfa Aesar, 15 molar equivalents) is added, followed by NaO^{t}Bu (AK Scientific, 1.5 molar equivalents). The reaction mixture is stirred overnight at 90° C. under N₂ atmosphere. Once the reaction reaches completion it is cooled to RT and the reaction mixture is partitioned between ethyl acetate and H₂O. The phases are separated, and the organic phase is partitioned a second time with H₂O, followed by brine. The organic layer is separated and dried over anhydrous sodium sulfate, filtered and concentrated under reduced pressure to afford the crude product.

The crude product is purified by flash silica column chromatography to afford the title compound.

### Preparation of 2-(1-benzhydrylpiperidin-4-yl)-6-(piperidin-1-yl)-1,2,3,4-tetrahydroisoquinoline (Compound 24)

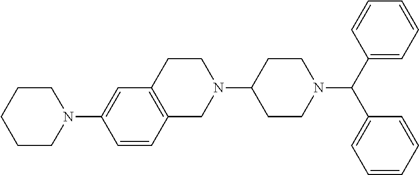 [SMILES: c1ccc(C(c2ccccc2)N2CCC(N3CCc4cc(N5CCCCC5)ccc4C3)CC2)cc1]

To a solution of Compound 1 (1.0 molar equivalents) in anhydrous toluene is added a pre-dissolved solution containing Pd₂(dba)₃ (Strem, 0.10 molar equivalents), and RuPhos (CombiBlocks, 0.20 molar equivalents) in toluene. Next, piperidine (Alfa Aesar, 15 molar equivalents) is added, followed by NaO^{t}Bu (AK Scientific, 1.5 molar equivalents). The reaction mixture is stirred overnight at 90° C. under N₂ atmosphere. Once the reaction reaches completion it is cooled to RT and the reaction mixture is partitioned between ethyl acetate and H₂O. The phases are separated, and the organic phase is partitioned a second time with H₂O, followed by brine. The organic layer is separated and dried over anhydrous sodium sulfate, filtered and concentrated under reduced pressure to afford the crude product. The crude product is purified by flash silica column chromatography to afford the title compound.

### Preparation of 2-(1-benzhydrylpiperidin-4-yl)-N,N-dipropyl-1,2,3,4-tetrahydroisoquinolin-6-amine (Compound 25)

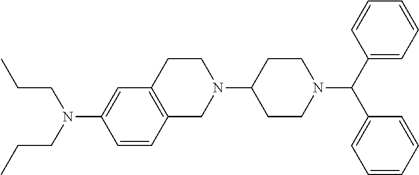 [SMILES: CCCN(CCC)c1ccc2c(c1)CCN(C1CCN(C(c3ccccc3)c3ccccc3)CC1)C2]

To a solution of Compound 1 (1.0 molar equivalents) in anhydrous toluene is added a pre-dissolved solution containing Pd₂(dba)₃ (Strem, 0.10 molar equivalents), and RuPhos (CombiBlocks, 0.20 molar equivalents) in toluene. Next, di-n-propylamine (Alfa Aesar, 15 molar equivalents) is added, followed by NaO^{t}Bu (AK Scientific, 1.5 molar equivalents). The reaction mixture is stirred overnight at 90° C. under N₂ atmosphere. Once the reaction reaches completion it is cooled to RT and the reaction mixture is partitioned between ethyl acetate and H₂O. The phases are separated, and the organic phase is partitioned a second time with H₂O, followed by brine. The organic layer is separated and dried over anhydrous sodium sulfate, filtered and concentrated under reduced pressure to afford the crude product. The crude product is purified by flash silica column chromatography to afford the title compound.

### Preparation of 2-(1-benzhydrylpiperidin-4-yl)-N,N-diisopropyl-1,2,3,4-tetrahydroisoquinolin-6-amine (Compound 26)

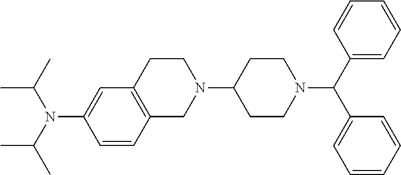 [SMILES: CC(C)N(c1ccc2c(c1)CCN(C1CCN(C(c3ccccc3)c3ccccc3)CC1)C2)C(C)C]

To a solution of Compound 1 (1.0 molar equivalents) in anhydrous toluene is added a pre-dissolved solution containing Pd₂(dba)₃ (Strem, 0.10 molar equivalents), and RuPhos (CombiBlocks, 0.20 molar equivalents) in toluene. Next, diisopropylamine (Alfa Aesar, 15 molar equivalents) is added, followed by NaO^{t}Bu (AK Scientific, 1.5 molar equivalents). The reaction mixture is stirred overnight at 90° C. under N₂ atmosphere. Once the reaction reaches completion it is cooled to RT and the reaction mixture is partitioned between ethyl acetate and H₂O. The phases are separated, and the organic phase is partitioned a second time with H₂O, followed by brine. The organic layer is separated and dried over anhydrous sodium sulfate, filtered and concentrated under reduced pressure to afford the crude product. The crude product is purified by flash silica column chromatography to afford the title compound.

### Preparation of 2-(1-benzhydrylpiperidin-4-yl)-N-methyl-1,2,3,4-tetrahydroisoquinolin-6-amine (Compound 27)

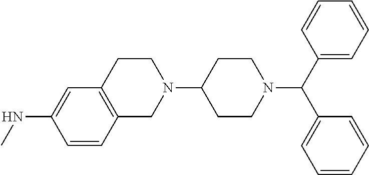 [SMILES: CNc1ccc2c(c1)CCN(C1CCN(C(c3ccccc3)c3ccccc3)CC1)C2]

To a solution of Compound 1 (1.0 molar equivalents) in anhydrous toluene is added a pre-dissolved solution containing Pd₂(dba)₃ (Strem, 0.10 molar equivalents), and RuPhos (CombiBlocks, 0.20 molar equivalents) in toluene. Next, methylamine (Alfa Aesar, 15 molar equivalents) is added, followed by NaO^{t}Bu (AK Scientific, 1.5 molar equivalents). The reaction mixture is stirred overnight at 90° C. under N₂ atmosphere. Once the reaction reaches completion it is cooled to RT and the reaction mixture is partitioned between ethyl acetate and H₂O. The phases are separated, and the organic phase is partitioned a second time with H₂O, followed by brine. The organic layer is separated and dried over anhydrous sodium sulfate, filtered and concentrated under reduced pressure to afford the crude product. The crude product is purified by flash silica column chromatography to afford the title compound.

### Preparation of 2-(1-benzhydrylpiperidin-4-yl)-N-ethyl-1,2,3,4-tetrahydroisoquinolin-6-amine (Compound 28)

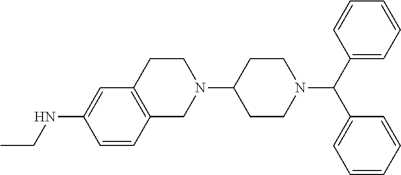 [SMILES: CCNc1ccc2c(c1)CCN(C1CCN(C(c3ccccc3)c3ccccc3)CC1)C2]

To a solution of Compound 1 (1.0 molar equivalents) in anhydrous toluene is added a pre-dissolved solution containing Pd₂(dba)₃ (Strem, 0.10 molar equivalents), and RuPhos (CombiBlocks, 0.20 molar equivalents) in toluene. Next, ethylamine (Alfa Aesar, 15 molar equivalents) is added, followed by NaO^{t}Bu (AK Scientific, 1.5 molar equivalents). The reaction mixture is stirred overnight at 90° C. under N₂ atmosphere. Once the reaction reaches completion it is cooled to RT and the reaction mixture is partitioned between ethyl acetate and H₂O. The phases are separated, and the organic phase is partitioned a second time with H₂O, followed by brine. The organic layer is separated and dried over anhydrous sodium sulfate, filtered and concentrated under reduced pressure to afford the crude product. The crude product is purified by flash silica column chromatography to afford the title compound.

### Preparation of 2-(1-benzhydrylpiperidin-4-yl)-N-isopropyl-1,2,3,4-tetrahydroisoquinolin-6-amine (Compound 29)

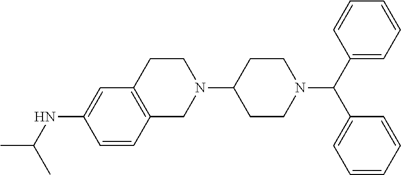 [SMILES: CC(C)Nc1ccc2c(c1)CCN(C1CCN(C(c3ccccc3)c3ccccc3)CC1)C2]

To a solution of Compound 1 (1.0 molar equivalents) in anhydrous toluene is added a pre-dissolved solution containing Pd₂(dba)₃ (Strem, 0.10 molar equivalents), and RuPhos (CombiBlocks, 0.20 molar equivalents) in toluene. Next, isopropylamine (Alfa Aesar, 15 molar equivalents) is added, followed by NaO^{t}Bu (AK Scientific, 1.5 molar equivalents). The reaction mixture is stirred overnight at 90° C. under N₂ atmosphere. Once the reaction reaches completion it is cooled to RT and the reaction mixture is partitioned between ethyl acetate and H₂O. The phases are separated, and the organic phase is partitioned a second time with H₂O, followed by brine. The organic layer is separated and dried over anhydrous sodium sulfate, filtered and concentrated under reduced pressure to afford the crude product. The crude product is purified by flash silica column chromatography to afford the title compound.

### Preparation of 2-(1-benzhydrylpiperidin-4-yl)-N-propyl-1,2,3,4-tetrahydroisoquinolin-6-amine (Compound 30)

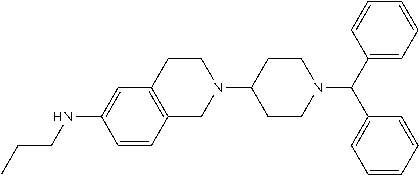 [SMILES: CCCNc1ccc2c(c1)CCN(C1CCN(C(c3ccccc3)c3ccccc3)CC1)C2]

To a solution of Compound 1 (1.0 molar equivalents) in anhydrous toluene is added a pre-dissolved solution containing Pd₂(dba)₃ (Strem, 0.10 molar equivalents), and RuPhos (CombiBlocks, 0.20 molar equivalents) in toluene. Next, 1-propylamine (Alfa Aesar, 15 molar equivalents) is added, followed by NaO^{t}Bu (AK Scientific, 1.5 molar equivalents). The reaction mixture is stirred overnight at 90° C. under N₂ atmosphere. Once the reaction reaches completion it is cooled to RT and the reaction mixture is partitioned between ethyl acetate and H₂O. The phases are separated, and the organic phase is partitioned a second time with H₂O, followed by brine. The organic layer is separated and dried over anhydrous sodium sulfate, filtered and concentrated under reduced pressure to afford the crude product. The crude product is purified by flash silica column chromatography to afford the title compound.

### Preparation of 2-(1-benzhydrylpiperidin-4-yl)-N-isobutyl-1,2,3,4-tetrahydroisoquinolin-6-amine (Compound 31)

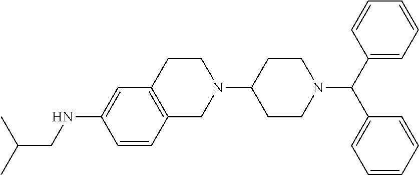 [SMILES: CC(C)CNc1ccc2c(c1)CCN(C1CCN(C(c3ccccc3)c3ccccc3)CC1)C2]

To a solution of Compound 1 (1.0 molar equivalents) in anhydrous toluene is added a pre-dissolved solution containing Pd₂(dba)₃ (Strem, 0.10 molar equivalents), and RuPhos (CombiBlocks, 0.20 molar equivalents) in toluene. Next, isobutylamine (Alfa Aesar, 15 molar equivalents) is added, followed by NaO^{t}Bu (AK Scientific, 1.5 molar equivalents). The reaction mixture is stirred overnight at 90° C. under N₂ atmosphere. Once the reaction reaches completion it is cooled to RT and the reaction mixture is partitioned between ethyl acetate and H₂O. The phases are separated, and the organic phase is partitioned a second time with H₂O, followed by brine. The organic layer is separated and dried over anhydrous sodium sulfate, filtered and concentrated under reduced pressure to afford the crude product. The crude product is purified by flash silica column chromatography to afford the title compound.

### Preparation of 2-(1-benzhydrylpiperidin-4-yl)-N,N-diisobutyl-1,2,3,4-tetrahydroisoquinolin-6-amine (Compound 32)

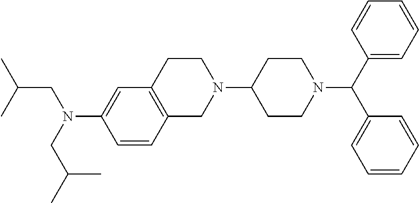 [SMILES: CC(C)CN(CC(C)C)c1ccc2c(c1)CCN(C1CCN(C(c3ccccc3)c3ccccc3)CC1)C2]

To a solution of Compound 1 (1.0 molar equivalents) in anhydrous toluene is added a pre-dissolved solution containing Pd₂(dba)₃ (Strem, 0.10 molar equivalents), and RuPhos (CombiBlocks, 0.20 molar equivalents) in toluene. Next, diisobutylamine (Alfa Aesar, 15 molar equivalents) is added, followed by NaO^{t}Bu (AK Scientific, 1.5 molar equivalents). The reaction mixture is stirred overnight at 90° C. under N₂ atmosphere. Once the reaction reaches completion it is cooled to RT and the reaction mixture is partitioned between ethyl acetate and H₂O. The phases are separated, and the organic phase is partitioned a second time with H₂O, followed by brine. The organic layer is separated and dried over anhydrous sodium sulfate, filtered and concentrated under reduced pressure to afford the crude product. The crude product is purified by flash silica column chromatography to afford the title compound.

### Preparation of 2-(1-benzhydrylpiperidin-4-yl)-6-(pyrrolidin-1-yl)-1,2,3,4-tetrahydroisoquinoline (Compound 33)

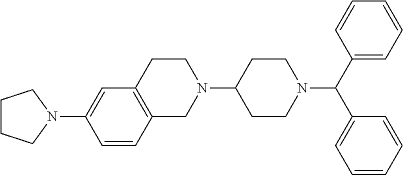 [SMILES: c1ccc(C(c2ccccc2)N2CCC(N3CCc4cc(N5CCCC5)ccc4C3)CC2)cc1]

To a solution of Compound 1 (1.0 molar equivalents) in anhydrous toluene is added a pre-dissolved solution containing Pd₂(dba)₃ (Strem, 0.10 molar equivalents), and RuPhos (CombiBlocks, 0.20 molar equivalents) in toluene. Next, pyrrolidine (Alfa Aesar, 15 molar equivalents) is added, followed by NaO^{t}Bu (AK Scientific, 1.5 molar equivalents). The reaction mixture is stirred overnight at 90° C. under N₂ atmosphere. Once the reaction reaches completion it is cooled to RT and the reaction mixture is partitioned between ethyl acetate and H₂O. The phases are separated, and the organic phase is partitioned a second time with H₂O, followed by brine. The organic layer is separated and dried over anhydrous sodium sulfate, filtered and concentrated under reduced pressure to afford the crude product. The crude product is purified by flash silica column chromatography to afford the title compound.

### Scheme 4: General Method for the Synthesis of 2-(1-Benzhydrylazetidin-3-yl)-1,2,3,4-Tetrahydroisoquinoline Derivatives (Compounds 34, 36-43) and 2-(1-Benzhydrylazetidin-3-yl)-1,2,3,4-tetrahydro-2,6-naphthyridine (Compound 35)

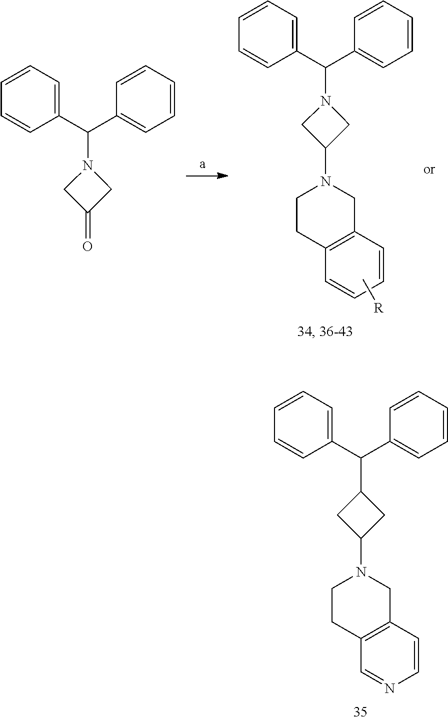

Reagents and conditions: (a) a-j, NaBH(OAc)₃, (THF/methanol/1,2-DCE), (TEA/acetic acid/both), 4-72 h, rt to 75° C. a=1,2,3,4-tetrahydroisoquinoline-6-carbonitrile hydrochloride; b=1,2,3,4-tetrahydro-2,6-naphthyridine hydrochloride; c=6-chloro-1,2,3,4-tetrahydroisoquinoline; d=6-fluoro-1,2,3,4-tetrahydroisoquinoline; e=5-fluoro-1,2,3,4-tetrahydroisoquinoline; f=7-fluoro-1,2,3,4-tetrahydroisoquinoline; g=8-fluoro-1,2,3,4-tetrahydroisoquinoline; h=6-(trifluoromethoxy)-1,2,3,4-tetrahydroisoquinoline hydrochloride; i=1,2,3,4-tetrahydro-2,7-naphthyridine; j=6-bromo-1,2,3,4-tetrahydroisoquinoline.

### Preparation of 2-(1-benzhydrylazetidin-3-yl)-1,2,3,4-tetrahydroisoquinoline-6-carbonitrile (Compound 34)

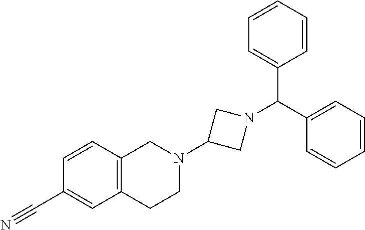 [SMILES: N#Cc1ccc2c(c1)CCN(C1CN(C(c3ccccc3)c3ccccc3)C1)C2]

To a mixture of 1-(1,1-diphenylmethyl)azetidin-3-one (Combi-Blocks, 1.2 molar equivalents) and 1,2,3,4-tetrahydroisoquinoline-6-carbonitrile hydrochloride (Aldrich, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 2-(1-benzhydrylazetidin-3-yl)-1,2,3,4-tetrahydro-2,6-naphthyridine (Compound 35)

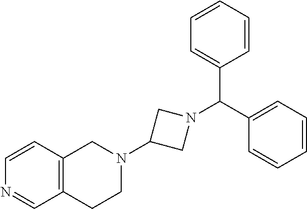 [SMILES: c1ccc(C(c2ccccc2)N2CC(N3CCc4cnccc4C3)C2)cc1]

To a mixture of 1-(1,1-diphenylmethyl)azetidin-3-one (Combi-Blocks, 1.2 molar equivalents) and 1,2,3,4-tetrahydro-2,6-naphthyridine hydrochloride (Combi-Blocks, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 2-(1-benzhydrylazetidin-3-yl)-6-chloro-1,2,3,4-tetrahydroisoquinoline (Compound 36)

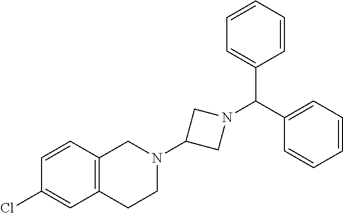 [SMILES: Clc1ccc2c(c1)CCN(C1CN(C(c3ccccc3)c3ccccc3)C1)C2]

To a mixture of 1-(1,1-diphenylmethyl)azetidin-3-one (Combi-Blocks, 1.2 molar equivalents) and 6-chloro-1,2,3,4-tetrahydroisoquinoline (Combi-Blocks, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 2-(1-benzhydrylazetidin-3-yl)-6-fluoro-1,2,3,4-tetrahydroisoquinoline (Compound 37)

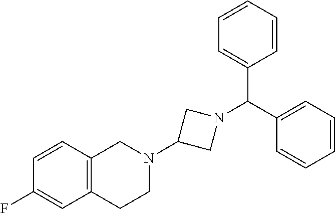 [SMILES: Fc1ccc2c(c1)CCN(C1CN(C(c3ccccc3)c3ccccc3)C1)C2]

To a mixture of 1-(1,1-diphenylmethyl)azetidin-3-one (Combi-Blocks, 1.2 molar equivalents) and 6-fluoro-1,2,3,4-tetrahydroisoquinoline (Enamine, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 2-(1-benzhydrylazetidin-3-yl)-5-fluoro-1,2,3,4-tetrahydroisoquinoline (Compound 38)

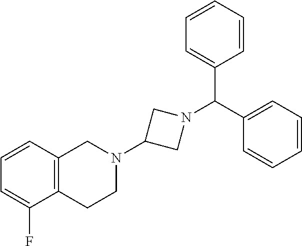 [SMILES: Fc1cccc2c1CCN(C1CN(C(c3ccccc3)c3ccccc3)C1)C2]

To a mixture of 1-(1,1-diphenylmethyl)azetidin-3-one (Combi-Blocks, 1.2 molar equivalents) and 5-fluoro-1,2,3,4-tetrahydroisoquinoline (Enamine, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 2-(1-benzhydrylazetidin-3-yl)-7-fluoro-1,2,3,4-tetrahydroisoquinoline (Compound 39)

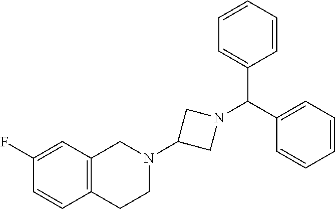 [SMILES: Fc1ccc2c(c1)CN(C1CN(C(c3ccccc3)c3ccccc3)C1)CC2]

To a mixture of 1-(1,1-diphenylmethyl)azetidin-3-one (Combi-Blocks, 1.2 molar equivalents) and 7-fluoro-1,2,3,4-tetrahydroisoquinoline (Enamine, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 2-(1-benzhydrylazetidin-3-yl)-8-fluoro-1,2,3,4-tetrahydroisoquinoline (Compound 40)

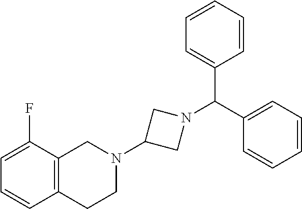 [SMILES: Fc1cccc2c1CN(C1CN(C(c3ccccc3)c3ccccc3)C1)CC2]

To a mixture of 1-(1,1-diphenylmethyl)azetidin-3-one (Combi-Blocks, 1.2 molar equivalents) and 8-fluoro-1,2,3,4-tetrahydroisoquinoline (Enamine, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 2-(1-benzhydrylazetidin-3-yl)-6-(trifluoromethoxy)-1,2,3,4-tetrahydroisoquinoline (Compound 41)

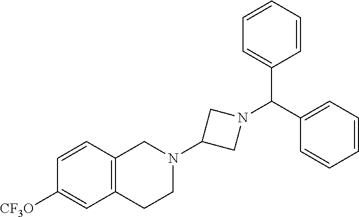

To a mixture of 1-(1,1-diphenylmethyl)azetidin-3-one (Combi-Blocks, 1.2 molar equivalents) and 6-(trifluoromethoxy)-1,2,3,4-tetrahydroisoquinoline hydrochloride (Enamine, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 2-(1-benzhydrylazetidin-3-yl)-1,2,3,4-tetrahydro-2,7-naphthyridine (Compound 42)

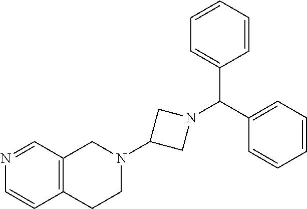 [SMILES: c1ccc(C(c2ccccc2)N2CC(N3CCc4ccncc4C3)C2)cc1]

To a mixture of 1-(1,1-diphenylmethyl)azetidin-3-one (Combi-Blocks, 1.2 molar equivalents) and 1,2,3,4-tetrahydro-2,7-naphthyridine hydrochloride (Enamine, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 2-(1-benzhydrylazetidin-3-yl)-6-bromo-1,2,3,4-tetrahydroisoquinoline (Compound 43)

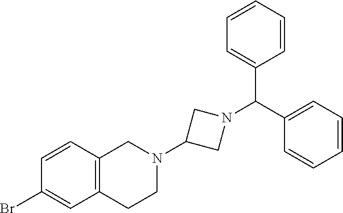 [SMILES: Brc1ccc2c(c1)CCN(C1CN(C(c3ccccc3)c3ccccc3)C1)C2]

To a mixture of 1-(1,1-diphenylmethyl)azetidin-3-one (Combi-Blocks, 0.475 g, 2.00 mmol) and 6-bromo-1,2,3,4-tetrahydroisoquinoline (Matrix Scientific, 0.212 g, 1.00 mmol) in 1,2-dichloroethane (15 mL) was added sodium triacetoxyborohydride (Arctom Chemicals, 0.511 g, 4.00 mmol). The reaction mixture was stirred for 4 hours at room temperature under N₂ atmosphere. Once the reaction reached completion, the reaction mixture was quenched by the addition of 2 ml of 10% NaOH solution. The reaction mixture was stirred for 10 minutes then 20 mL of saturated NaHCO₃ was added. The mixture was poured into a separatory funnel and the organic layer was separated. The aqueous layer was extracted with DCM (2×25 mL), and the combined organic layers were dried over anhydrous sodium sulfate, filtered, and concentrated under reduced pressure to give the crude product. The crude product was purified by flash silica column chromatography on a CombiFlash NextGen 300+ purification system. Elution through a 24 g RediSep Gold Rf flash silica cartridge with 0-10% methanol in dichloromethane afforded the title compound as a yellow oil that solidified upon standing (0.433 g, 87%); Rf 0.58 with 95:5 v/v dichloromethane-methanol (UV); MS (APCI⁺) m/z 433.1, 435.1 (M+1). HPLC UV purity, Rt=8.752 min, 98.76%.

### Scheme 5: General Method for the Synthesis of 2-(1-Benzhydrylazetidin-3-yl)-1,2,3,4-tetrahydroisoquinolin-6-amine Derivatives (Compounds 32-44) from 2-(1-Benzhydrylazetidin-3-yl)-6-bromo-1,2,3,4-tetrahydroisoquinoline (Compound 31)

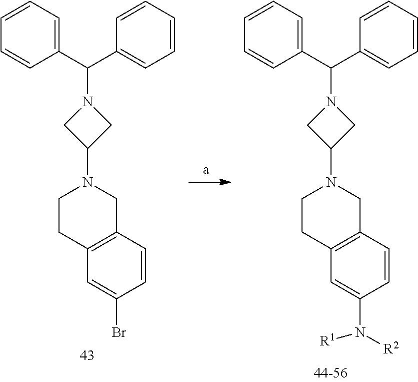

Reagents and conditions: (a) Pd₂(dba)₃, a-m, RuPhos, NaO^{t}Bu, toluene, 90° C., 16 h. a=dimethylamine; b=morpholine; c=piperidine; d=di-n-propylamine; e=diisopropylamine; f=methylamine; g=ethylamine; h=isopropylamine; i=1-propylamine; j=isobutylamine; k=diisobutylamine; I=pyrrolidine; m=diethylamine.

### Preparation of 2-(1-benzhydrylazetidin-3-yl)-N,N-dimethyl-1,2,3,4-tetrahydroisoquinolin-6-amine (Compound 44)

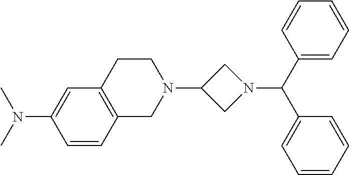 [SMILES: CN(C)c1ccc2c(c1)CCN(C1CN(C(c3ccccc3)c3ccccc3)C1)C2]

To a solution of Compound 43 (1.0 molar equivalents) in anhydrous toluene is added a pre-dissolved solution containing Pd₂(dba)₃ (Strem, 0.10 molar equivalents), and RuPhos (CombiBlocks, 0.20 molar equivalents) in toluene. Next, dimethylamine (Alfa Aesar, 15 molar equivalents) is added, followed by NaO^{t}Bu (AK Scientific, 1.5 molar equivalents). The reaction mixture is stirred overnight at 90° C. under N₂ atmosphere. Once the reaction reaches completion it is cooled to RT and the reaction mixture is partitioned between ethyl acetate and H₂O. The phases are separated, and the organic phase is partitioned a second time with H₂O, followed by brine. The organic layer is separated and dried over anhydrous sodium sulfate, filtered and concentrated under reduced pressure to afford the crude product. The crude product is purified by flash silica column chromatography to afford the title compound.

### Preparation of 4-(2-(1-benzhydrylazetidin-3-yl)-1,2,3,4-tetrahydroisoquinolin-6-yl)morpholine (Compound 45)

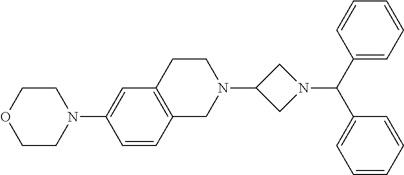 [SMILES: c1ccc(C(c2ccccc2)N2CC(N3CCc4cc(N5CCOCC5)ccc4C3)C2)cc1]

To a solution of Compound 43 (1.0 molar equivalents) in anhydrous toluene is added a pre-dissolved solution containing Pd₂(dba)₃ (Strem, 0.10 molar equivalents), and RuPhos (CombiBlocks, 0.20 molar equivalents) in toluene. Next, morpholine (Alfa Aesar, 15 molar equivalents) is added, followed by NaO^{t}Bu (AK Scientific, 1.5 molar equivalents). The reaction mixture is stirred overnight at 90° C. under N₂ atmosphere. Once the reaction reaches completion it is cooled to RT and the reaction mixture is partitioned between ethyl acetate and H₂O. The phases are separated, and the organic phase is partitioned a second time with H₂O, followed by brine. The organic layer is separated and dried over anhydrous sodium sulfate, filtered and concentrated under reduced pressure to afford the crude product. The crude product is purified by flash silica column chromatography to afford the title compound.

### Preparation of 2-(1-benzhydrylazetidin-3-yl)-6-(piperidin-1-yl)-1,2,3,4-tetrahydroisoquinoline (Compound 46)

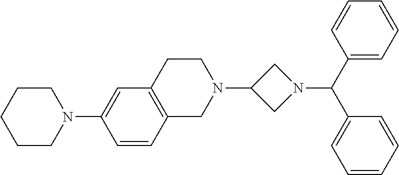 [SMILES: c1ccc(C(c2ccccc2)N2CC(N3CCc4cc(N5CCCCC5)ccc4C3)C2)cc1]

To a solution of Compound 43 (1.0 molar equivalents) in anhydrous toluene is added a pre-dissolved solution containing Pd₂(dba)₃ (Strem, 0.10 molar equivalents), and RuPhos (CombiBlocks, 0.20 molar equivalents) in toluene. Next, piperidine (Alfa Aesar, 15 molar equivalents) is added, followed by NaO^{t}Bu (AK Scientific, 1.5 molar equivalents). The reaction mixture is stirred overnight at 90° C. under N₂ atmosphere. Once the reaction reaches completion it is cooled to RT and the reaction mixture is partitioned between ethyl acetate and H₂O. The phases are separated, and the organic phase is partitioned a second time with H₂O, followed by brine. The organic layer is separated and dried over anhydrous sodium sulfate, filtered and concentrated under reduced pressure to afford the crude product. The crude product is purified by flash silica column chromatography to afford the title compound.

### Preparation of 2-(1-benzhydrylazetidin-3-yl)-N,N-dipropyl-1,2,3,4-tetrahydroisoquinolin-6-amine (Compound 47)

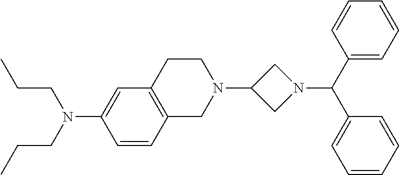 [SMILES: CCCN(CCC)c1ccc2c(c1)CCN(C1CN(C(c3ccccc3)c3ccccc3)C1)C2]

To a solution of Compound 43 (1.0 molar equivalents) in anhydrous toluene is added a pre-dissolved solution containing Pd₂(dba)₃ (Strem, 0.10 molar equivalents), and RuPhos (CombiBlocks, 0.20 molar equivalents) in toluene. Next, di-n-propylamine (Alfa Aesar, 15 molar equivalents) is added, followed by NaO^{t}Bu (AK Scientific, 1.5 molar equivalents). The reaction mixture is stirred overnight at 90° C. under N₂ atmosphere. Once the reaction reaches completion it is cooled to RT and the reaction mixture is partitioned between ethyl acetate and H₂O. The phases are separated, and the organic phase is partitioned a second time with H₂O, followed by brine. The organic layer is separated and dried over anhydrous sodium sulfate, filtered and concentrated under reduced pressure to afford the crude product. The crude product is purified by flash silica column chromatography to afford the title compound.

### Preparation of 2-(1-benzhydrylazetidin-3-yl)-N,N-diisopropyl-1,2,3,4-tetrahydroisoquinolin-6-amine (Compound 48)

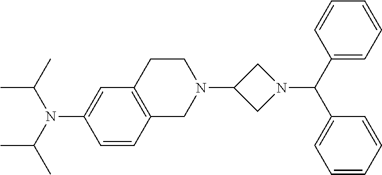 [SMILES: CC(C)N(c1ccc2c(c1)CCN(C1CN(C(c3ccccc3)c3ccccc3)C1)C2)C(C)C]

To a solution of Compound 43 (1.0 molar equivalents) in anhydrous toluene is added a pre-dissolved solution containing Pd₂(dba)₃ (Strem, 0.10 molar equivalents), and RuPhos (CombiBlocks, 0.20 molar equivalents) in toluene. Next, diisopropylamine (Alfa Aesar, 15 molar equivalents) is added, followed by NaO^{t}Bu (AK Scientific, 1.5 molar equivalents). The reaction mixture is stirred overnight at 90° C. under N₂ atmosphere. Once the reaction reaches completion it is cooled to RT and the reaction mixture is partitioned between ethyl acetate and H₂O. The phases are separated, and the organic phase is partitioned a second time with H₂O, followed by brine. The organic layer is separated and dried over anhydrous sodium sulfate, filtered and concentrated under reduced pressure to afford the crude product. The crude product is purified by flash silica column chromatography to afford the title compound.

### Preparation of 2-(1-benzhydrylazetidin-3-yl)-N-methyl-1,2,3,4-tetrahydroisoquinolin-6-amine (Compound 49)

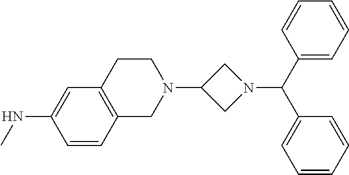 [SMILES: CNc1ccc2c(c1)CCN(C1CN(C(c3ccccc3)c3ccccc3)C1)C2]

To a solution of Compound 43 (1.0 molar equivalents) in anhydrous toluene is added a pre-dissolved solution containing Pd₂(dba)₃ (Strem, 0.10 molar equivalents), and RuPhos (CombiBlocks, 0.20 molar equivalents) in toluene. Next, methylamine (Alfa Aesar, 15 molar equivalents) is added, followed by NaO^{t}Bu (AK Scientific, 1.5 molar equivalents). The reaction mixture is stirred overnight at 90° C. under N₂ atmosphere. Once the reaction reaches completion it is cooled to RT and the reaction mixture is partitioned between ethyl acetate and H₂O. The phases are separated, and the organic phase is partitioned a second time with H₂O, followed by brine. The organic layer is separated and dried over anhydrous sodium sulfate, filtered and concentrated under reduced pressure to afford the crude product. The crude product is purified by flash silica column chromatography to afford the title compound.

### Preparation of 2-(1-benzhydrylazetidin-3-yl)-N-ethyl-1,2,3,4-tetrahydroisoquinolin-6-amine (Compound 50)

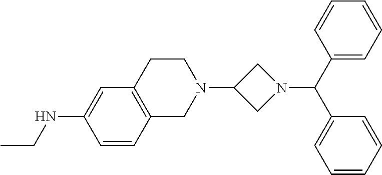 [SMILES: CCNc1ccc2c(c1)CCN(C1CN(C(c3ccccc3)c3ccccc3)C1)C2]

To a solution of Compound 43 (1.0 molar equivalents) in anhydrous toluene is added a pre-dissolved solution containing Pd₂(dba)₃ (Strem, 0.10 molar equivalents), and RuPhos (CombiBlocks, 0.20 molar equivalents) in toluene. Next, ethylamine (Alfa Aesar, 15 molar equivalents) is added, followed by NaO^{t}Bu (AK Scientific, 1.5 molar equivalents). The reaction mixture is stirred overnight at 90° C. under N₂ atmosphere. Once the reaction reaches completion it is cooled to RT and the reaction mixture is partitioned between ethyl acetate and H₂O. The phases are separated, and the organic phase is partitioned a second time with H₂O, followed by brine. The organic layer is separated and dried over anhydrous sodium sulfate, filtered and concentrated under reduced pressure to afford the crude product. The crude product is purified by flash silica column chromatography to afford the title compound.

### Preparation of 2-(1-benzhydrylazetidin-3-yl)-N-isopropyl-1,2,3,4-tetrahydroisoquinolin-6-amine (Compound 51)

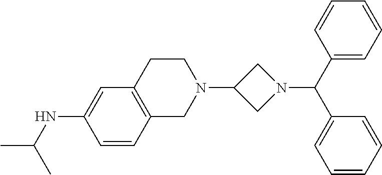 [SMILES: CC(C)Nc1ccc2c(c1)CCN(C1CN(C(c3ccccc3)c3ccccc3)C1)C2]

To a solution of Compound 43 (1.0 molar equivalents) in anhydrous toluene is added a pre-dissolved solution containing Pd₂(dba)₃ (Strem, 0.10 molar equivalents), and RuPhos (CombiBlocks, 0.20 molar equivalents) in toluene. Next, isopropylamine (Alfa Aesar, 15 molar equivalents) is added, followed by NaO^{t}Bu (AK Scientific, 1.5 molar equivalents). The reaction mixture is stirred overnight at 90° C. under N₂ atmosphere. Once the reaction reaches completion it is cooled to RT and the reaction mixture is partitioned between ethyl acetate and H₂O. The phases are separated, and the organic phase is partitioned a second time with H₂O, followed by brine. The organic layer is separated and dried over anhydrous sodium sulfate, filtered and concentrated under reduced pressure to afford the crude product. The crude product is purified by flash silica column chromatography to afford the title compound.

### Preparation of 2-(1-benzhydrylazetidin-3-yl)-N-propyl-1,2,3,4-tetrahydroisoquinolin-6-amine (Compound 52)

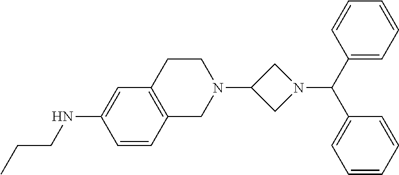 [SMILES: CCCNc1ccc2c(c1)CCN(C1CN(C(c3ccccc3)c3ccccc3)C1)C2]

To a solution of Compound 43 (1.0 molar equivalents) in anhydrous toluene is added a pre-dissolved solution containing Pd₂(dba)₃ (Strem, 0.10 molar equivalents), and RuPhos (CombiBlocks, 0.20 molar equivalents) in toluene. Next, 1-propylamine (Alfa Aesar, 15 molar equivalents) is added, followed by NaO^{t}Bu (AK Scientific, 1.5 molar equivalents). The reaction mixture is stirred overnight at 90° C. under N₂ atmosphere. Once the reaction reaches completion it is cooled to RT and the reaction mixture is partitioned between ethyl acetate and H₂O. The phases are separated, and the organic phase is partitioned a second time with H₂O, followed by brine. The organic layer is separated and dried over anhydrous sodium sulfate, filtered and concentrated under reduced pressure to afford the crude product. The crude product is purified by flash silica column chromatography to afford the title compound.

### Preparation of 2-(1-benzhydrylazetidin-3-yl)-N-isobutyl-1,2,3,4-tetrahydroisoquinolin-6-amine (Compound 53)

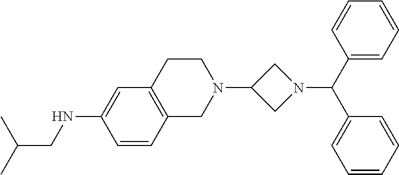 [SMILES: CC(C)CNc1ccc2c(c1)CCN(C1CN(C(c3ccccc3)c3ccccc3)C1)C2]

To a solution of Compound 43 (1.0 molar equivalents) in anhydrous toluene is added a pre-dissolved solution containing Pd₂(dba)₃ (Strem, 0.10 molar equivalents), and RuPhos (CombiBlocks, 0.20 molar equivalents) in toluene. Next, isobutylamine (Alfa Aesar, 15 molar equivalents) is added, followed by NaO^{t}Bu (AK Scientific, 1.5 molar equivalents). The reaction mixture is stirred overnight at 90° C. under N₂ atmosphere. Once the reaction reaches completion it is cooled to RT and the reaction mixture is partitioned between ethyl acetate and H₂O. The phases are separated, and the organic phase is partitioned a second time with H₂O, followed by brine. The organic layer is separated and dried over anhydrous sodium sulfate, filtered and concentrated under reduced pressure to afford the crude product. The crude product is purified by flash silica column chromatography to afford the title compound.

### Preparation of 2-(1-benzhydrylazetidin-3-yl)-N,N-diisobutyl-1,2,3,4-tetrahydroisoquinolin-6-amine (Compound 54)

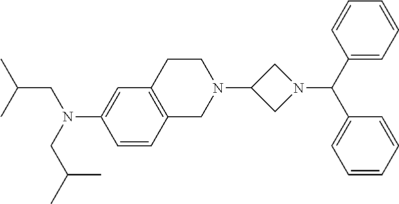 [SMILES: CC(C)CN(CC(C)C)c1ccc2c(c1)CCN(C1CN(C(c3ccccc3)c3ccccc3)C1)C2]

To a solution of Compound 43 (1.0 molar equivalents) in anhydrous toluene is added a pre-dissolved solution containing Pd₂(dba)₃ (Strem, 0.10 molar equivalents), and RuPhos (CombiBlocks, 0.20 molar equivalents) in toluene. Next, diisobutylamine (Alfa Aesar, 15 molar equivalents) is added, followed by NaO^{t}Bu (AK Scientific, 1.5 molar equivalents). The reaction mixture is stirred overnight at 90° C. under N₂ atmosphere. Once the reaction reaches completion it is cooled to RT and the reaction mixture is partitioned between ethyl acetate and H₂O. The phases are separated, and the organic phase is partitioned a second time with H₂O, followed by brine. The organic layer is separated and dried over anhydrous sodium sulfate, filtered and concentrated under reduced pressure to afford the crude product. The crude product is purified by flash silica column chromatography to afford the title compound.

### Preparation of 2-(1-benzhydrylazetidin-3-yl)-6-(pyrrolidin-1-yl)-1,2,3,4-tetrahydroisoquinoline (Compound 55)

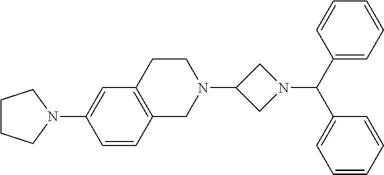 [SMILES: c1ccc(C(c2ccccc2)N2CC(N3CCc4cc(N5CCCC5)ccc4C3)C2)cc1]

To a solution of Compound 43 (1.0 molar equivalents) in anhydrous toluene is added a pre-dissolved solution containing Pd₂(dba)₃ (Strem, 0.10 molar equivalents), and RuPhos (CombiBlocks, 0.20 molar equivalents) in toluene. Next, pyrrolidine (Alfa Aesar, 15 molar equivalents) is added, followed by NaO^{t}Bu (AK Scientific, 1.5 molar equivalents). The reaction mixture is stirred overnight at 90° C. under N₂ atmosphere. Once the reaction reaches completion it is cooled to RT and the reaction mixture is partitioned between ethyl acetate and H₂O. The phases are separated, and the organic phase is partitioned a second time with H₂O, followed by brine. The organic layer is separated and dried over anhydrous sodium sulfate, filtered and concentrated under reduced pressure to afford the crude product. The crude product is purified by flash silica column chromatography to afford the title compound.

### Preparation of 2-(1-benzhydrylazetidin-3-yl)-N,N-diethyl-1,2,3,4-tetrahydroisoquinolin-6-amine (Compound 56)

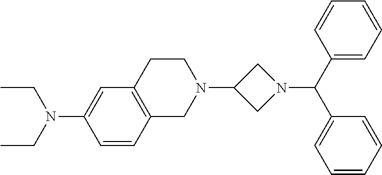 [SMILES: CCN(CC)c1ccc2c(c1)CCN(C1CN(C(c3ccccc3)c3ccccc3)C1)C2]

To a solution of Compound 31 (0.433 g, 1.0 mmol) in anhydrous toluene (60 mL) was added a pre-dissolved solution containing Pd₂(dba)₃ (Strem, 0.183 g, 0.20 mmol), and RuPhos (CombiBlocks, 0.186 g, 0.40 mmol) in toluene (10 mL). Next, diethylamine (Alfa Aesar, 2 mL, 20 mmol) was added, followed by NaO^{t}Bu (AK Scientific, 0.144 g, 1.5 mmol). The reaction mixture was stirred for 5 hours at 120° C. under N₂ atmosphere. Once the reaction reached completion, it was cooled to RT, and the reaction mixture was partitioned between ethyl acetate (200 mL) and H₂O (200 mL). The phases were separated, and the organic phase is partitioned a second time with H₂O (150 mL), followed by brine (150 mL). The organic layer was separated and dried over anhydrous sodium sulfate, filtered and concentrated under reduced pressure to afford the crude product. The crude material was purified by flash silica column chromatography on a CombiFlash NextGen 300+ purification system. Elution through a 24 g RediSep Gold Rf flash silica cartridge with 0-10% methanol in dichloromethane afforded the title compound as an orange oil that solidified upon standing (0.433 g, 87%); R_{f} 0.60 with 50:50 v/v hexanes-ethyl acetate (UV); ¹H-NMR (400 MHz; CDCl₃) δ 7.4-7.5 (m, 4H), 7.3-7.4 (m, 1H), 7.1-7.2 (m, 2H), 7.03 (dd, 1H, J=5.7, 8.4 Hz), 6.82 (dt, 1H, J=2.7, 8.6 Hz), 6.72 (dd, 1H, J=2.7, 9.4 Hz), 4.26 (s, 1H), 3.78 (s, 2 h), 2.98 (br d, 2H, J=11.3 Hz), 2.85 (s, 4H), 2.49 (m, 1H), 1.7-1.9 (m, 5H); MS (APCI⁺) m/z 424.20 (M+1). HPLC UV purity, Rt=9.022 min, 98.95%; melting point=54-56° C.

### Scheme 6: General Method for the Synthesis of 3-(1-Benzhydrylpiperidin-4-yl)-2,3,4,5-tetrahydro-1H-benzo[d]azepine Derivatives (Compounds 57-64) and 7-(1-Benzhydrylpiperidin-4-yl)-6,7,8,9-tetrahydro-5H-pyrido[3,4-d]azepine (Compound 65)

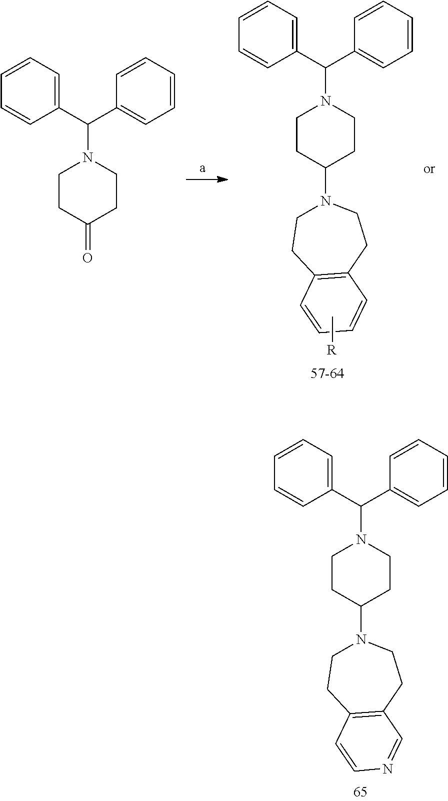

Reagents and conditions: (a) a-i, NaBH(OAc)₃, (THF/methanol/1,2-DCE), (TEA/acetic acid/both), 4-72 h, rt to 75° C. a=2,3,4,5-tetrahydro-1H-3-benzazepine; b=7-methoxy-2,3,4,5-tetrahydro-1H-3-benzazepine; c=2,3,4,5-tetrahydro-1H-3-benzazepin-7-ol; d=7-nitro-2,3,4,5-tetrahydro-1H-benzo[d]azepine; e=7-fluoro-2,3,4,5-tetrahydro-1H-3-benzazepine; f=7-chloro-2,3,4,5-tetrahydro-1H-3-benzazepine; g=7-bromo-2,3,4,5-tetrahydro-1H-3-benzazepine; h=7-cyano-2,3,4,5-tetrahydro-1H-3-benzazepine; i=6,7,8,9-tetrahydro-5H-pyrido[3,4-d]azepine.

### Preparation of 3-(1-benzhydrylpiperidin-4-yl)-2,3,4,5-tetrahydro-1H-benzo[d]azepine (Compound 57)

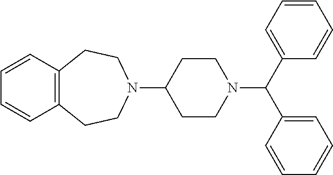 [SMILES: c1ccc(C(c2ccccc2)N2CCC(N3CCc4ccccc4CC3)CC2)cc1]

To a mixture of 1-benzhydrylpiperidin-4-one (BLDpharm, 1.2 molar equivalents) and 2,3,4,5-tetrahydro-1H-benzo[d]azepine (Enamine, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 3-(1-benzhydrylpiperidin-4-yl)-7-methoxy-2,3,4,5-tetrahydro-1H-benzo[d]azepine (Compound 58)

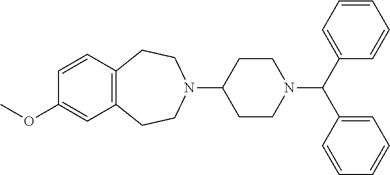 [SMILES: COc1ccc2c(c1)CCN(C1CCN(C(c3ccccc3)c3ccccc3)CC1)CC2]

To a mixture of 1-benzhydrylpiperidin-4-one (BLDpharm, 1.2 molar equivalents) and 7-methoxy-2,3,4,5-tetrahydro-1H-3-benzazepine (Enamine, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 3-(1-benzhydrylpiperidin-4-yl)-2,3,4,5-tetrahydro-1H-benzo[d]azepin-7-ol (Compound 59)

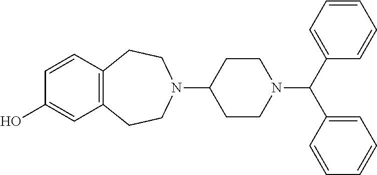 [SMILES: Oc1ccc2c(c1)CCN(C1CCN(C(c3ccccc3)c3ccccc3)CC1)CC2]

To a mixture of 1-benzhydrylpiperidin-4-one (BLDpharm, 1.2 molar equivalents) and 2,3,4,5-tetrahydro-1H-3-benzazepin-7-ol (Enamine, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 3-(1-benzhydrylpiperidin-4-yl)-7-nitro-2,3,4,5-tetrahydro-1H-benzo[d]azepine (Compound 60)

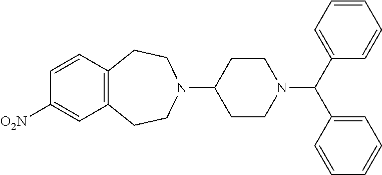 [SMILES: O=[N+]([O-])c1ccc2c(c1)CCN(C1CCN(C(c3ccccc3)c3ccccc3)CC1)CC2]

To a mixture of 1-benzhydrylpiperidin-4-one (BLDpharm, 1.2 molar equivalents) and 7-nitro-2,3,4,5-tetrahydro-1H-benzo[d]azepine (Enamine, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 3-(1-benzhydrylpiperidin-4-yl)-7-fluoro-2,3,4,5-tetrahydro-1H-benzo[d]azepine (Compound 61)

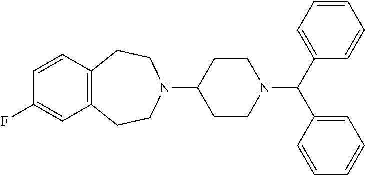 [SMILES: Fc1ccc2c(c1)CCN(C1CCN(C(c3ccccc3)c3ccccc3)CC1)CC2]

To a mixture of 1-benzhydrylpiperidin-4-one (BLDpharm, 1.2 molar equivalents) and 7-fluoro-2,3,4,5-tetrahydro-1H-3-benzazepine (Matrix, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 3-(1-benzhydrylpiperidin-4-yl)-7-chloro-2,3,4,5-tetrahydro-1H-benzo[d]azepine (Compound 62)

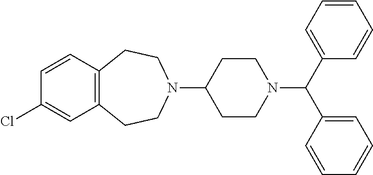 [SMILES: Clc1ccc2c(c1)CCN(C1CCN(C(c3ccccc3)c3ccccc3)CC1)CC2]

To a mixture of 1-benzhydrylpiperidin-4-one (BLDpharm, 1.2 molar equivalents) and 7-chloro-2,3,4,5-tetrahydro-1H-3-benzazepine (Enamine, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 3-(1-benzhydrylpiperidin-4-yl)-7-bromo-2,3,4,5-tetrahydro-1H-benzo[d]azepine (Compound 63)

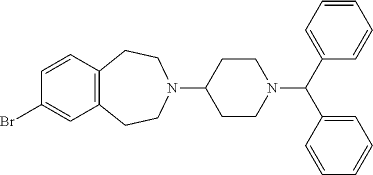 [SMILES: Brc1ccc2c(c1)CCN(C1CCN(C(c3ccccc3)c3ccccc3)CC1)CC2]

To a mixture of 1-benzhydrylpiperidin-4-one (BLDpharm, 1.2 molar equivalents) and 7-bromo-2,3,4,5-tetrahydro-1H-3-benzazepine (Enamine, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 3-(1-benzhydrylpiperidin-4-yl)-2,3,4,5-tetrahydro-1H-benzo[d]azepine-7-carbonitrile (Compound 64)

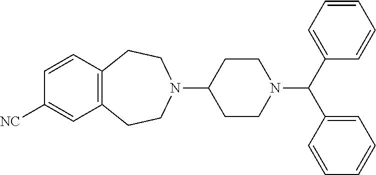 [SMILES: N#Cc1ccc2c(c1)CCN(C1CCN(C(c3ccccc3)c3ccccc3)CC1)CC2]

To a mixture of 1-benzhydrylpiperidin-4-one (BLDpharm, 1.2 molar equivalents) and 7-cyano-2,3,4,5-tetrahydro-1H-3-benzazepine (AbaChemScene, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 7-(1-benzhydrylpiperidin-4-yl)-6,7,8,9-tetrahydro-5H-pyrido[3,4-d]azepine (Compound 65)

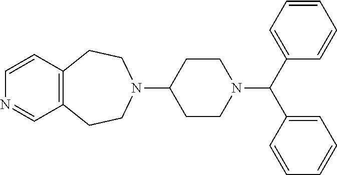 [SMILES: c1ccc(C(c2ccccc2)N2CCC(N3CCc4ccncc4CC3)CC2)cc1]

To a mixture of 1-benzhydrylpiperidin-4-one (BLDpharm, 1.2 molar equivalents) and 6,7,8,9-tetrahydro-5H-pyrido[3,4-d]azepine (A Chem Block, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Scheme 7: General Method for the Synthesis of 2-(1-Benzhydrylpiperidin-4-yl)isoindoline Derivatives (Compounds 66-73 and 75-76), and 7-(1-Benzhydrylpiperidin-4-yl)-6,7,8,9-tetrahydro-5H-pyrido[3,4-d]azepine (Compound 74)

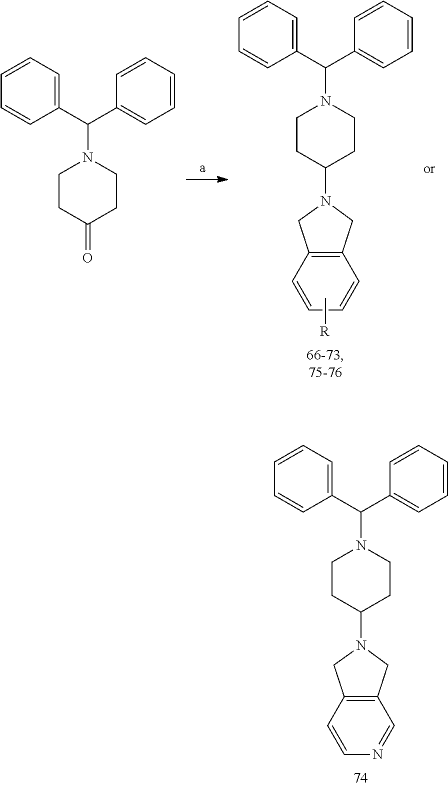

Reagents and conditions: (a) a-k, NaBH(OAc)₃, (THF/methanol/1,2-DCE), (TEA/Acetic Acid/Both), 4-72 h, rt to 75° C. a=isoindoline; b=5-methoxyisoindoline; c=2,3-dihydro-1H-isoindol-5-ol; d=2,3-dihydro-5-nitro-1H-isoindole; e=5-fluoro-2,3-dihydro-1H-isoindole; f=5-chloro-2,3-dihydro-1H-isoindole; g=5-bromo-2,3-dihydro-1H-isoindole; h=5-cyano-2,3-dihydro-1H-isoindole; i=1H,2H,3H-pyrrolo[3,4-c]pyridine; j=N,N-diethyl-2,3-dihydro-1H-isoindol-5-amine; k=4-fluoro-2,3-dihydro-1H-isoindole.

### Preparation of 2-(1-benzhydrylpiperidin-4-yl)isoindoline (Compound 66)

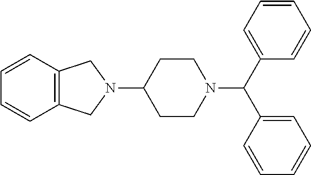 [SMILES: c1ccc(C(c2ccccc2)N2CCC(N3Cc4ccccc4C3)CC2)cc1]

To a mixture of 1-benzhydrylpiperidin-4-one (BLDpharm, 1.2 molar equivalents) and isoindoline (Aldrich, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 2-(1-benzhydrylpiperidin-4-yl)-5-methoxyisoindoline (Compound 67)

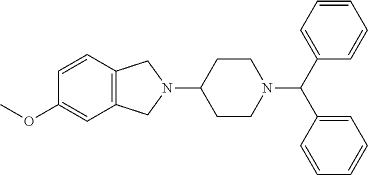 [SMILES: COc1ccc2c(c1)CN(C1CCN(C(c3ccccc3)c3ccccc3)CC1)C2]

To a mixture of 1-benzhydrylpiperidin-4-one (BLDpharm, 1.2 molar equivalents) and 5-methoxyisoindoline (Enamine, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 2-(1-benzhydrylpiperidin-4-yl)isoindolin-5-ol (Compound 68)

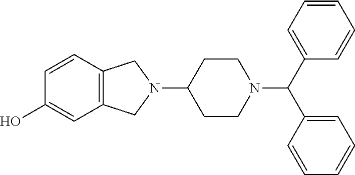 [SMILES: Oc1ccc2c(c1)CN(C1CCN(C(c3ccccc3)c3ccccc3)CC1)C2]

To a mixture of 1-benzhydrylpiperidin-4-one (BLDpharm, 1.2 molar equivalents) and 2,3-dihydro-1H-isoindol-5-ol (Matrix, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 2-(1-benzhydrylpiperidin-4-yl)-5-nitroisoindoline (Compound 69)

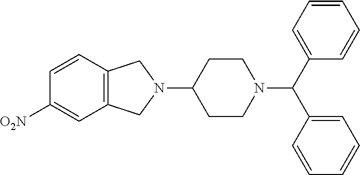 [SMILES: O=[N+]([O-])c1ccc2c(c1)CN(C1CCN(C(c3ccccc3)c3ccccc3)CC1)C2]

To a mixture of 1-benzhydrylpiperidin-4-one (BLDpharm, 1.2 molar equivalents) and 2,3-dihydro-5-nitro-1H-isoindole (Enamine, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 2-(1-benzhydrylpiperidin-4-yl)-5-fluoroisoindoline (Compound 70)

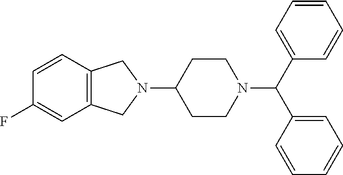 [SMILES: Fc1ccc2c(c1)CN(C1CCN(C(c3ccccc3)c3ccccc3)CC1)C2]

To a mixture of 1-benzhydrylpiperidin-4-one (BLDpharm, 1.2 molar equivalents) and 5-fluoro-2,3-dihydro-1H-isoindole (Enamine, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 2-(1-benzhydrylpiperidin-4-yl)-5-chloroisoindoline (Compound 71)

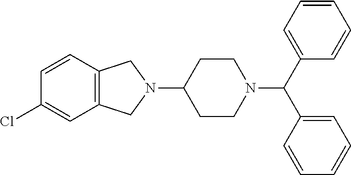 [SMILES: Clc1ccc2c(c1)CN(C1CCN(C(c3ccccc3)c3ccccc3)CC1)C2]

To a mixture of 1-benzhydrylpiperidin-4-one (BLDpharm, 1.2 molar equivalents) and 5-chloro-2,3-dihydro-1H-isoindole (Enamine, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 2-(1-benzhydrylpiperidin-4-yl)-5-bromoisoindoline (Compound 72)

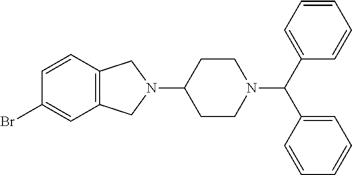 [SMILES: Brc1ccc2c(c1)CN(C1CCN(C(c3ccccc3)c3ccccc3)CC1)C2]

To a mixture of 1-benzhydrylpiperidin-4-one (BLDpharm, 1.2 molar equivalents) and 5-bromo-2,3-dihydro-1H-isoindole (Enamine, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 2-(1-benzhydrylpiperidin-4-yl)isoindoline-5-carbonitrile (Compound 73)

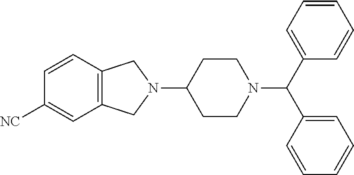 [SMILES: N#Cc1ccc2c(c1)CN(C1CCN(C(c3ccccc3)c3ccccc3)CC1)C2]

To a mixture of 1-benzhydrylpiperidin-4-one (BLDpharm, 1.2 molar equivalents) and 5-cyano-2,3-dihydro-1H-isoindole (Enamine, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 2-(1-benzhydrylpiperidin-4-yl)-2,3-dihydro-1H-pyrrolo[3,4-c]pyridine (Compound 74)

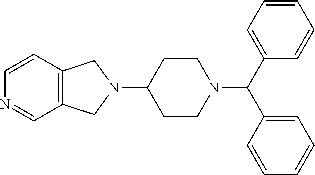 [SMILES: c1ccc(C(c2ccccc2)N2CCC(N3Cc4ccncc4C3)CC2)cc1]

To a mixture of 1-benzhydrylpiperidin-4-one (BLDpharm, 1.2 molar equivalents) and 1H,2H,3H-pyrrolo[3,4-c]pyridine (Enamine, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 2-(1-benzhydrylpiperidin-4-yl)-N,N-diethylisoindolin-5-amin (Compound 75)

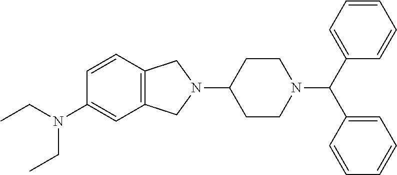 [SMILES: CCN(CC)c1ccc2c(c1)CN(C1CCN(C(c3ccccc3)c3ccccc3)CC1)C2]

To a mixture of 1-benzhydrylpiperidin-4-one (BLDpharm, 1.2 molar equivalents) and N,N-diethyl-2,3-dihydro-1H-isoindol-5-amine (Aurora Fine Chemicals, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Preparation of 2-(1-benzhydrylpiperidin-4-yl)-4-fluoroisoindoline (Compound 76)

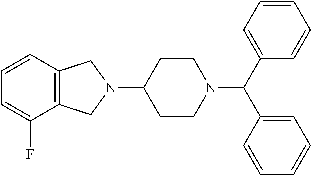 [SMILES: Fc1cccc2c1CN(C1CCN(C(c3ccccc3)c3ccccc3)CC1)C2]

To a mixture of 1-benzhydrylpiperidin-4-one (BLDpharm, 1.2 molar equivalents) and 4-fluoro-2,3-dihydro-1H-isoindole (Enamine, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

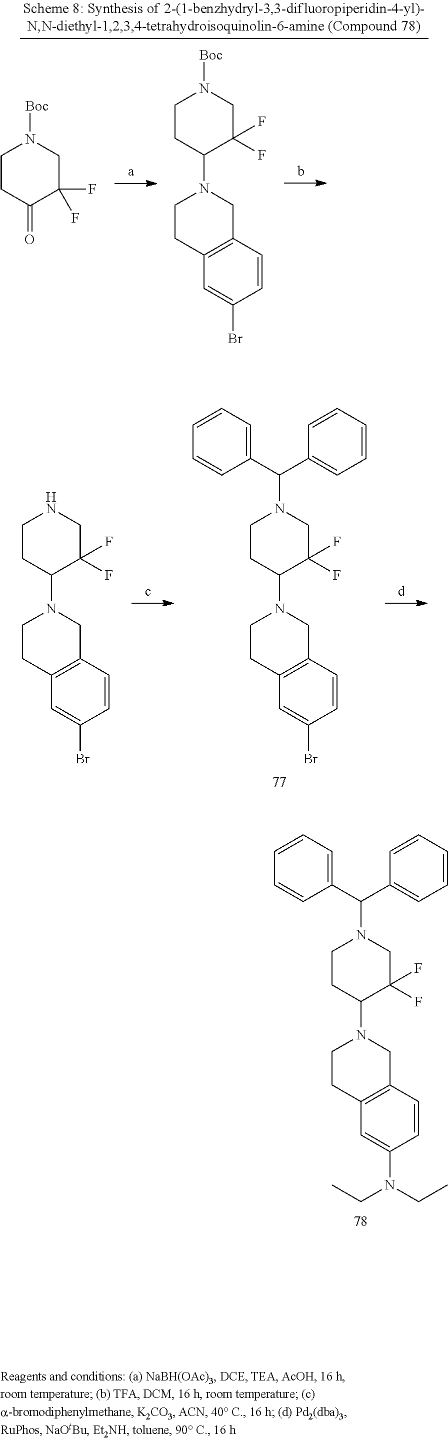 [SMILES: CC(C)(C)OC(=O)N1CCC(=O)C(F)(F)C1 ; CC(C)(C)OC(=O)N1CCC(N2CCc3cc(Br)ccc3C2)C(F)(F)C1 ; FC1(F)CNCCC1N1CCc2cc(Br)ccc2C1 ; FC1(F)CN(C(c2ccccc2)c2ccccc2)CCC1N1CCc2cc(Br)ccc2C1 ; CCN(CC)c1ccc2c(c1)CCN(C1CCN(C(c3ccccc3)c3ccccc3)CC1(F)F)C2]

### Step a: Preparation of tert-butyl 4-(6-bromo-3,4-dihydroisoquinolin-2(1H)-yl)-3,3-difluoropiperidine-1-carboxylate

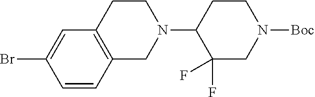 [SMILES: CC(C)(C)OC(=O)N1CCC(N2CCc3cc(Br)ccc3C2)C(F)(F)C1]

To a mixture of 3,3-difluoro-4-piperidinone hydrochloride (AChemBlock, 1.2 molar equivalents) and 6-bromo-1,2,3,4-tetrahydroisoquinoline (Matrix Scientific, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Step b: Preparation of 6-bromo-2-(3,3-difluoropiperidin-4-yl)-1,2,3,4-tetrahydroisoquinoline

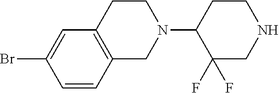 [SMILES: FC1(F)CNCCC1N1CCc2cc(Br)ccc2C1]

To a solution of tert-butyl 4-(6-bromo-3,4-dihydroisoquinolin-2(1H)-yl)-3,3-difluoropiperidine-1-carboxylate (1.0 molar equivalents) in DCM is added TFA (10.0 molar equivalents). The reaction mixture is stirred overnight at room temperature under N₂ atmosphere. Once the reaction mixture reaches completion the crude solution is concentrated under reduced pressure in the presence of toluene. The crude product is purified by flash silica column chromatography to afford the title compound.

### Step c: Preparation of 2-(1-benzhydryl-3,3-difluoropiperidin-4-yl)-6-bromo-1,2,3,4-tetrahydroisoquinoline (Compound 77)

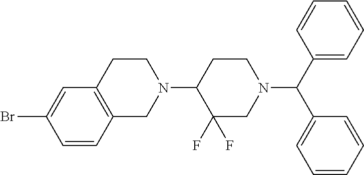 [SMILES: FC1(F)CN(C(c2ccccc2)c2ccccc2)CCC1N1CCc2cc(Br)ccc2C1]

To a solution of 6-bromo-2-(3,3-difluoropiperidin-4-yl)-1,2,3,4-tetrahydroisoquinoline (1.0 molar equivalents) in ACN is added K₂CO₃ (VWR, 2.1 molar equivalents), followed by addition of α-bromodiphenylmethane (TCl, 1.05 molar equivalents). The reaction mixture is stirred overnight at 40° C. under N₂ atmosphere. Once the reaction reaches completion it is cooled to RT. Next, the crude reaction mixture is partitioned with H₂O. The phases are separated, and the organic phase is partitioned with brine. The organic layer is separated and concentrated under reduced pressure to afford the crude product. The crude product is purified by flash silica column chromatography to afford the title compound.

### Step d: Preparation of 2-(1-benzhydryl-3,3-difluoropiperidin-4-yl)-N,N-diethyl-1,2,3,4-tetrahydroisoquinolin-6-amine (Compound 78)

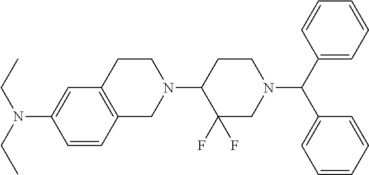 [SMILES: CCN(CC)c1ccc2c(c1)CCN(C1CCN(C(c3ccccc3)c3ccccc3)CC1(F)F)C2]

To a solution of Compound 77 (1.0 molar equivalents) in anhydrous toluene is added a pre-dissolved solution containing Pd₂(dba)₃ (Strem, 0.10 molar equivalents), and RuPhos (CombiBlocks, 0.20 molar equivalents) in toluene. Next, diethylamine (Alfa Aesar, 15 molar equivalents) is added, followed by NaO^{t}Bu (AK Scientific, 1.5 molar equivalents). The reaction mixture is stirred overnight at 90° C. under N₂ atmosphere. Once the reaction reaches completion it is cooled to RT and the reaction mixture is partitioned between ethyl acetate and H₂O. The phases are separated, and the organic phase is partitioned a second time with H₂O, followed by brine. The organic layer is separated and dried over anhydrous sodium sulfate, filtered and concentrated under reduced pressure to afford the crude product. The crude product is purified by flash silica column chromatography to afford the title compound.

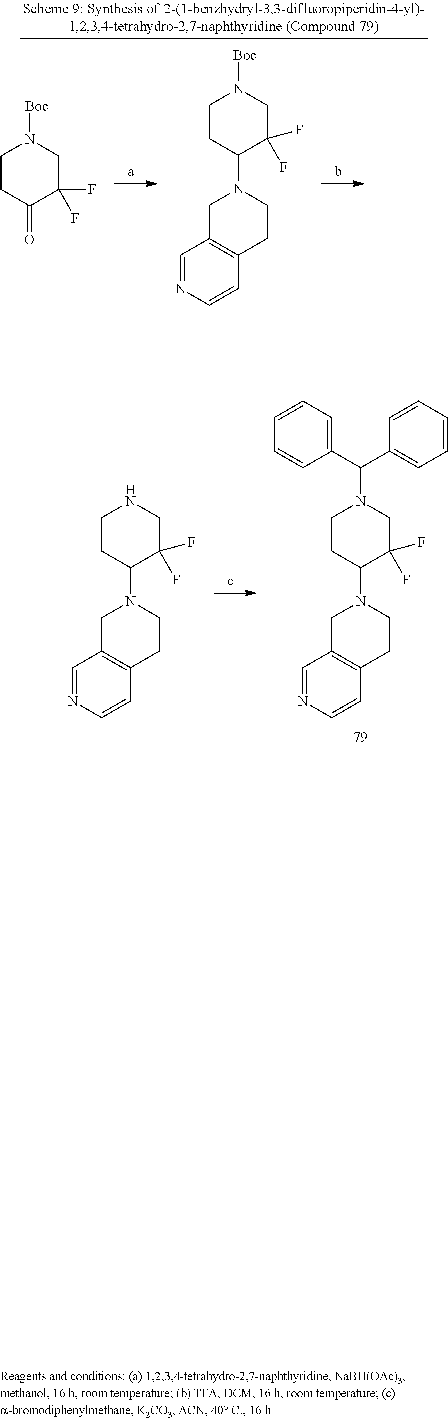 [SMILES: CC(C)(C)OC(=O)N1CCC(=O)C(F)(F)C1 ; CC(C)(C)OC(=O)N1CCC(N2CCc3ccncc3C2)C(F)(F)C1 ; FC1(F)CNCCC1N1CCc2ccncc2C1 ; FC1(F)CN(C(c2ccccc2)c2ccccc2)CCC1N1CCc2ccncc2C1]

### Step a: Preparation of tert-butyl 4-(3,4-dihydro-2,7-naphthyridin-2(1H)-yl)-3,3-difluoropiperidine-1-carboxylate

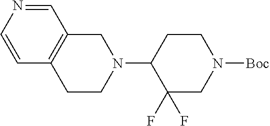 [SMILES: CC(C)(C)OC(=O)N1CCC(N2CCc3ccncc3C2)C(F)(F)C1]

To a mixture of 3,3-difluoro-4-piperidinone hydrochloride (AChemBlock, 1.2 molar equivalents) and 1,2,3,4-tetrahydro-2,7-naphthyridine (Matrix Scientific, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Step b: Preparation of 2-(3,3-difluoropiperidin-4-yl)-1,2,3,4-tetrahydro-2,7-naphthyridine

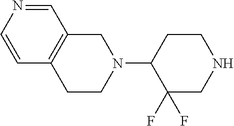 [SMILES: FC1(F)CNCCC1N1CCc2ccncc2C1]

To a solution of tert-butyl 4-(3,4-dihydro-2,7-naphthyridin-2(1H)-yl)-3,3-difluoropiperidine-1-carboxylate (1.0 molar equivalents) in DCM is added TFA (10.0 molar equivalents). The reaction mixture is stirred overnight at room temperature under N₂ atmosphere. Once the reaction mixture reaches completion the crude solution is concentrated under reduced pressure in the presence of toluene. The crude product is purified by flash silica column chromatography to afford the title compound.

### Step c: Preparation of 2-(1-benzhydryl-3,3-difluoropiperidin-4-yl)-1,2,3,4-tetrahydro-2,7-naphthyridine (Compound 79)

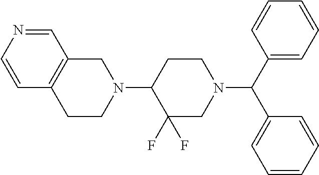 [SMILES: FC1(F)CN(C(c2ccccc2)c2ccccc2)CCC1N1CCc2ccncc2C1]

To a solution of 2-(3,3-difluoropiperidin-4-yl)-1,2,3,4-tetrahydro-2,7-naphthyridine (1.0 molar equivalents) in ACN is added K₂CO₃ (VWR, 2.1 molar equivalents), followed by addition of α-bromodiphenylmethane (TCl, 1.05 molar equivalents). The reaction mixture is stirred overnight at 40° C. under N₂ atmosphere. Once the reaction reaches completion it is cooled to RT. Next, the crude reaction mixture is partitioned with H₂O. The phases are separated, and the organic phase is partitioned with brine. The organic layer is separated and concentrated under reduced pressure to afford the crude product. The crude product is purified by flash silica column chromatography to afford the title compound.

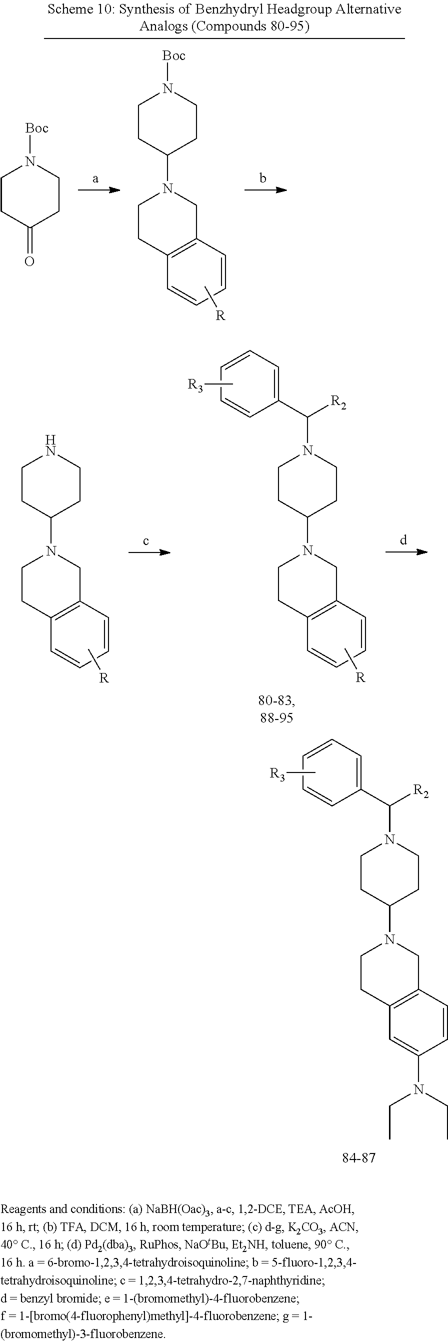

### Preparation of 2-(1-benzylpiperidin-4-yl)-6-bromo-1,2,3,4-tetrahydroisoquinoline (Compound 80)

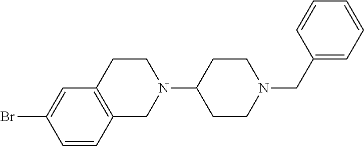 [SMILES: Brc1ccc2c(c1)CCN(C1CCN(Cc3ccccc3)CC1)C2]

### Step a: Preparation of tert-butyl 4-(6-bromo-3,4-dihydroisoquinolin-2(1H)-yl)piperidine-1-carboxylate

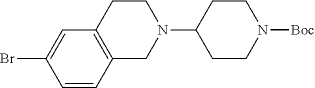 [SMILES: CC(C)(C)OC(=O)N1CCC(N2CCc3cc(Br)ccc3C2)CC1]

To a mixture of 1-Boc-4-piperidinone (Chem Impex, 1.2 molar equivalents) and 6-bromo-1,2,3,4-tetrahydroisoquinoline (Matrix Scientific, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Step b: Preparation of 6-bromo-2-(piperidin-4-yl)-1,2,3,4-tetrahydroisoquinoline

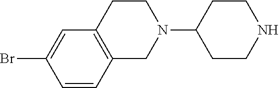 [SMILES: Brc1ccc2c(c1)CCN(C1CCNCC1)C2]

To a solution of tert-butyl 4-(6-bromo-3,4-dihydroisoquinolin-2(1H)-yl)piperidine-1-carboxylate (1.0 molar equivalents) in DCM is added TFA (10.0 molar equivalents). The reaction mixture is stirred overnight at room temperature under N₂ atmosphere. Once the reaction mixture reaches completion the crude solution is concentrated under reduced pressure in the presence of toluene. The crude product is purified by flash silica column chromatography to afford the title compound.

### Step c: Preparation of 2-(1-benzylpiperidin-4-yl)-6-bromo-1,2,3,4-tetrahydroisoquinoline (Compound 80)

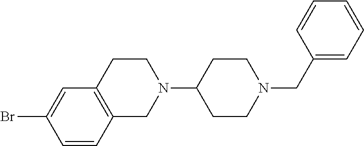 [SMILES: Brc1ccc2c(c1)CCN(C1CCN(Cc3ccccc3)CC1)C2]

To a solution of 6-bromo-2-(piperidin-4-yl)-1,2,3,4-tetrahydroisoquinoline (1.0 molar equivalents) in ACN is added K₂CO₃ (VWR, 2.1 molar equivalents), followed by addition of benzyl bromide (TCl, 1.05 molar equivalents). The reaction mixture is stirred overnight at 40° C. under N₂ atmosphere. Once the reaction reaches completion it is cooled to RT. Next, the crude reaction mixture is partitioned with H₂O.

The phases are separated, and the organic phase is partitioned with brine. The organic layer is separated and concentrated under reduced pressure to afford the crude product. The crude product is purified by flash silica column chromatography to afford the title compound.

### Preparation of 6-bromo-2-(1-(4-fluorobenzyl)piperidin-4-yl)-1,2,3,4-tetrahydroisoquinoline (Compound 81)

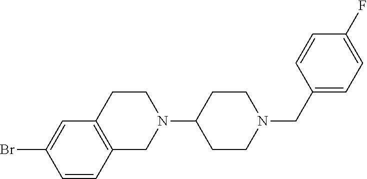 [SMILES: Fc1ccc(CN2CCC(N3CCc4cc(Br)ccc4C3)CC2)cc1]

To a solution of 6-bromo-2-(piperidin-4-yl)-1,2,3,4-tetrahydroisoquinoline (0.435 g, 2.00 mmol) in ACN (50 mL) was added triethylamine (VWR, 0.4 mL, 2.40 mmol), followed by addition of 1-(bromomethyl)-4-fluorobenzene hydrochloride (Aldrich, 0.500 g, 2.00 mmol) and sodium triacetoxyborohydride (Arctom Chemicals, 0.500 g, 4.00 mmol). The reaction mixture was stirred for three hours at room temperature under N₂ atmosphere. Once the reaction reached completion, the reaction mixture was quenched with the addition of 10% NaOH (1.5 mL). Next, the reaction mixture was partitioned between ethyl acetate (15 mL) and sat. aq. NaHCO₃ (15 mL). The phases are separated, and the aqueous phase was extracted with dichloromethane (2×20 mL). The combined organic layers was dried over anhydrous sodium sulfate, filtered and concentrated under reduced pressure to give the crude product. The crude product was purified by flash silica column chromatography on a CombiFlash NextGen 300+ purification system. Elution through a 24 g RediSep Gold Rf flash silica cartridge with 1:99% (10% TEA in methanol):dichloromethane afforded the title compound as a clear yellow oil (0.447 g, 55% yield); Rf 0.57 with 10:90 v/v methanol-dichloromethane (UV); ¹H-NMR (400 MHz; CDCl₃) δ 7.3-7.4 (m, 2H), 7.2-7.3 (m, 2H), 6.89 (d, 1H, J=8.2 Hz), 3.72 (s, 2H), 3.54 (br s, 2H), 3.00 (br d, 2H, J=10.9 Hz), 2.86 (br d, 2H, J=5.1 Hz), 2.83 (d, 2H, J=4.7 Hz), 2.5-2.6 (m, 1H), 2.0-2.2 (m, 2H), 1.7-1.9 (m, 4H); MS (APCI⁺) m/z 403.10 (M+1); HPLC UV purity, Rt=7.050 min, 98.53%.

### Preparation of 2-(1-(bis(4-fluorophenyl)methyl)piperidin-4-yl)-6-bromo-1,2,3,4-tetrahydroisoquinoline (Compound 82)

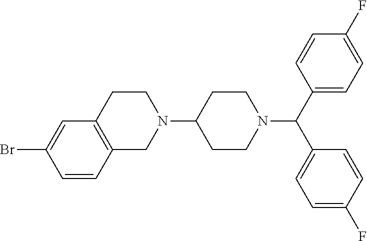 [SMILES: Fc1ccc(C(c2ccc(F)cc2)N2CCC(N3CCc4cc(Br)ccc4C3)CC2)cc1]

To a solution of 6-bromo-2-(piperidin-4-yl)-1,2,3,4-tetrahydroisoquinoline (1.0 molar equivalents) in ACN is added K₂CO₃ (VWR, 2.1 molar equivalents), followed by addition of 1-[bromo(4-fluorophenyl)methyl]-4-fluorobenzene (CombiBlocks, 1.05 molar equivalents). The reaction mixture is stirred overnight at 40° C. under N₂ atmosphere. Once the reaction reaches completion it is cooled to RT. Next, the crude reaction mixture is partitioned with H₂O. The phases are separated, and the organic phase is partitioned with brine. The organic layer is separated and concentrated under reduced pressure to afford the crude product. The crude product is purified by flash silica column chromatography to afford the title compound.

### Preparation of 6-bromo-2-(1-(3-fluorobenzyl)piperidin-4-yl)-1,2,3,4-tetrahydroisoquinoline (Compound 83)

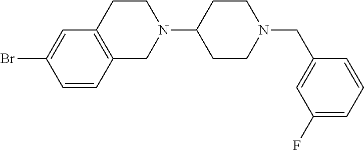 [SMILES: Fc1cccc(CN2CCC(N3CCc4cc(Br)ccc4C3)CC2)c1]

To a solution of 6-bromo-2-(piperidin-4-yl)-1,2,3,4-tetrahydroisoquinoline (1.0 molar equivalents) in ACN is added K₂CO₃ (VWR, 2.1 molar equivalents), followed by addition of 1-(bromomethyl)-3-fluorobenzene (Aldrich, 1.05 molar equivalents). The reaction mixture is stirred overnight at 40° C. under N₂ atmosphere. Once the reaction reaches completion it is cooled to RT. Next, the crude reaction mixture is partitioned with H₂O. The phases are separated, and the organic phase is partitioned with brine. The organic layer is separated and concentrated under reduced pressure to afford the crude product. The crude product is purified by flash silica column chromatography to afford the title compound.

### Preparation of 2-(1-benzylpiperidin-4-yl)-N,N-diethyl-1,2,3,4-tetrahydroisoquinolin-6-amine (Compound 84)

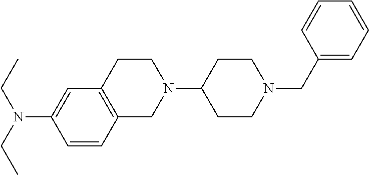 [SMILES: CCN(CC)c1ccc2c(c1)CCN(C1CCN(Cc3ccccc3)CC1)C2]

To a solution of Compound 80 (1.0 molar equivalents) in anhydrous toluene is added a pre-dissolved solution containing Pd₂(dba)₃ (Strem, 0.10 molar equivalents), and RuPhos (CombiBlocks, 0.20 molar equivalents) in toluene. Next, diethylamine (Alfa Aesar, 15 molar equivalents) is added, followed by NaO^{t}Bu (AK Scientific, 1.5 molar equivalents). The reaction mixture is stirred overnight at 90° C. under N₂ atmosphere. Once the reaction reaches completion it is cooled to RT and the reaction mixture is partitioned between ethyl acetate and H₂O. The phases are separated, and the organic phase is partitioned a second time with H₂O, followed by brine. The organic layer is separated and dried over anhydrous sodium sulfate, filtered and concentrated under reduced pressure to afford the crude product. The crude product is purified by flash silica column chromatography to afford the title compound.

Preparation of N,N-diethyl-2-(1-(4-fluorobenzyl)piperidin-4-yl)-1,2,3,4-tetrahydroisoquinolin-6-amine (Compound 85)

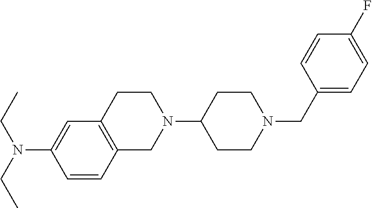 [SMILES: CCN(CC)c1ccc2c(c1)CCN(C1CCN(Cc3ccc(F)cc3)CC1)C2]

To a solution of Compound 81 (0.410 g, 1.0 mmol) in anhydrous toluene (60 mL) was added a pre-dissolved solution containing Pd₂(dba)₃ (Strem, 0.185 g, 0.203 mmol), and RuPhos (CombiBlocks, 0.189 g, 0.406 mmol) in toluene (3 mL). Next, diethylamine (Alfa Aesar, 3 mL, 30 mmol) was added, followed by NaO^{t}Bu (AK Scientific, 0.144 g, 1.50 mmol). The reaction mixture was stirred at 120° C. under N₂ atmosphere for 5 hours. Once the reaction reached completion it was cooled to RT and the reaction mixture was then concentrated under reduced pressure. The crude solid was purified by flash silica column chromatography on a CombiFlash NextGen 300+ purification system. Elution through a 24 g RediSep Gold Rf flash silica cartridge with 1-10% methanol in dichloromethane afforded the title compound as yellow waxy solid (0.085 g, 21% yield); R_{f} 0.43 with 10:90 v/v methanol-dichloromethane (UV); ¹H-NMR (400 MHz; DMSO-d₆) δ 7.34 (br t, 2H, J=6.8 Hz), 7.15 (t, 2H, J=8.8 Hz), 6.86 (br d, 1H, J=8.6 Hz), 6.49 (br d, 1H, J=9.0 Hz), 6.39 (br s, 1H), 3.2-3.3 (m, 7H), 2.7-2.9 (m, 5H), 1.8-2.1 (m, 4H), 1.6-1.7 (m, 2H), 1.04 (t, 6H, J=6.8 Hz); MS (APCI⁺) m/z 396.3 (M+1); HPLC UV purity, Rt=6.742 min, 96.11%; melting point=106-108° C.

### Preparation of 2-(1-(bis(4-fluorophenyl)methyl)piperidin-4-yl)-N,N-diethyl-1,2,3,4-tetrahydroisoquinolin-6-amine (Compound 86)

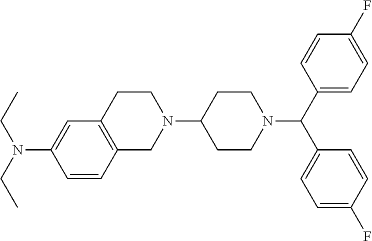 [SMILES: CCN(CC)c1ccc2c(c1)CCN(C1CCN(C(c3ccc(F)cc3)c3ccc(F)cc3)CC1)C2]

To a solution of Compound 82 (1.0 molar equivalents) in anhydrous toluene is added a pre-dissolved solution containing Pd₂(dba)₃ (Strem, 0.10 molar equivalents), and RuPhos (CombiBlocks, 0.20 molar equivalents) in toluene. Next, diethylamine (Alfa Aesar, 15 molar equivalents) is added, followed by NaO^{t}Bu (AK Scientific, 1.5 molar equivalents). The reaction mixture is stirred overnight at 90° C. under N₂ atmosphere. Once the reaction reaches completion it is cooled to RT and the reaction mixture is partitioned between ethyl acetate and H₂O. The phases are separated, and the organic phase is partitioned a second time with H₂O, followed by brine. The organic layer is separated and dried over anhydrous sodium sulfate, filtered and concentrated under reduced pressure to afford the crude product. The crude product is purified by flash silica column chromatography to afford the title compound.

### Preparation of N,N-diethyl-2-(1-(3-fluorobenzyl)piperidin-4-yl)-1,2,3,4-tetrahydroisoquinolin-6-amine (Compound 87)

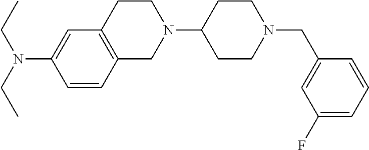 [SMILES: CCN(CC)c1ccc2c(c1)CCN(C1CCN(Cc3cccc(F)c3)CC1)C2]

To a solution of Compound 83 (1.0 molar equivalents) in anhydrous toluene is added a pre-dissolved solution containing Pd₂(dba)₃ (Strem, 0.10 molar equivalents), and RuPhos (CombiBlocks, 0.20 molar equivalents) in toluene. Next, diethylamine (Alfa Aesar, 15 molar equivalents) is added, followed by NaO^{t}Bu (AK Scientific, 1.5 molar equivalents). The reaction mixture is stirred overnight at 90° C. under N₂ atmosphere. Once the reaction reaches completion it is cooled to RT and the reaction mixture is partitioned between ethyl acetate and H₂O. The phases are separated, and the organic phase is partitioned a second time with H₂O, followed by brine. The organic layer is separated and dried over anhydrous sodium sulfate, filtered and concentrated under reduced pressure to afford the crude product. The crude product is purified by flash silica column chromatography to afford the title compound.

### Preparation of 2-(1-benzylpiperidin-4-yl)-5-fluoro-1,2,3,4-tetrahydroisoquinoline (Compound 88)

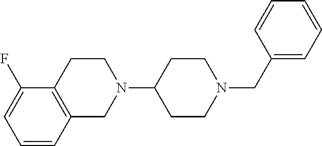 [SMILES: Fc1cccc2c1CCN(C1CCN(Cc3ccccc3)CC1)C2]

### Step a: Preparation of tert-butyl 4-(5-fluoro-3,4-dihydroisoquinolin-2(1H)-yl)piperidine-1-carboxylate

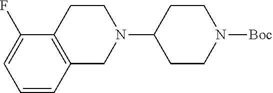 [SMILES: CC(C)(C)OC(=O)N1CCC(N2CCc3c(F)cccc3C2)CC1]

To a mixture of 1-Boc-4-piperidinone (Chem Impex, 1.2 molar equivalents) and 5-fluoro-1,2,3,4-tetrahydroisoquinoline (Matrix Scientific, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Step b: Preparation of 5-fluoro-2-(piperidin-4-yl)-1,2,3,4-tetrahydroisoquinoline

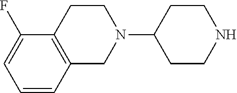 [SMILES: Fc1cccc2c1CCN(C1CCNCC1)C2]

To a solution of tert-butyl 4-(5-fluoro-3,4-dihydroisoquinolin-2(1H)-yl)piperidine-1-carboxylate (1.0 molar equivalents) in DCM is added TFA (10.0 molar equivalents). The reaction mixture is stirred overnight at room temperature under N₂ atmosphere. Once the reaction mixture reaches completion the crude solution is concentrated under reduced pressure in the presence of toluene. The crude product is purified by flash silica column chromatography to afford the title compound.

### Step c: Preparation of 2-(1-benzylpiperidin-4-yl)-5-fluoro-1,2,3,4-tetrahydroisoquinoline tetrahydroisoquinoline (Compound 88)

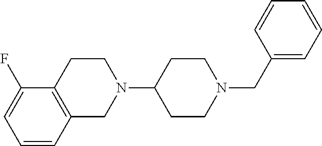 [SMILES: Fc1cccc2c1CCN(C1CCN(Cc3ccccc3)CC1)C2]

To a solution of 5-fluoro-2-(piperidin-4-yl)-1,2,3,4-tetrahydroisoquinoline (1.0 molar equivalents) in ACN is added K₂CO₃ (VWR, 2.1 molar equivalents), followed by addition of benzyl bromide (Aldrich, 1.05 molar equivalents). The reaction mixture is stirred overnight at 40° C. under N₂ atmosphere. Once the reaction reaches completion it is cooled to RT. Next, the crude reaction mixture is partitioned with H₂O. The phases are separated, and the organic phase is partitioned with brine. The organic layer is separated and concentrated under reduced pressure to afford the crude product. The crude product is purified by flash silica column chromatography to afford the title compound.

### Preparation of 5-fluoro-2-(1-(4-fluorobenzyl)piperidin-4-yl)-1,2,3,4-tetrahydroisoquinoline (Compound 89)

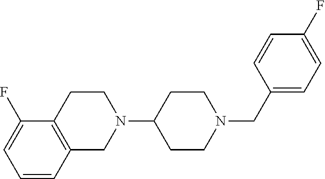 [SMILES: Fc1ccc(CN2CCC(N3CCc4c(F)cccc4C3)CC2)cc1]

To a solution of 5-fluoro-2-(piperidin-4-yl)-1,2,3,4-tetrahydroisoquinoline (1.0 molar equivalents) in ACN is added K₂CO₃ (VWR, 2.1 molar equivalents), followed by addition of 1-(bromomethyl)-4-fluorobenzene (Aldrich, 1.05 molar equivalents). The reaction mixture is stirred overnight at 40° C. under N₂ atmosphere. Once the reaction reaches completion it is cooled to RT. Next, the crude reaction mixture is partitioned with H₂O. The phases are separated, and the organic phase is partitioned with brine. The organic layer is separated and concentrated under reduced pressure to afford the crude product. The crude product is purified by flash silica column chromatography to afford the title compound.

### Preparation of 2-(1-(bis(4-fluorophenyl)methyl)piperidin-4-yl)-5-fluoro-1,2,3,4-tetrahydroisoquinoline (Compound 90)

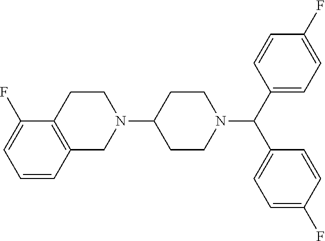 [SMILES: Fc1ccc(C(c2ccc(F)cc2)N2CCC(N3CCc4c(F)cccc4C3)CC2)cc1]

To a solution of 5-fluoro-2-(piperidin-4-yl)-1,2,3,4-tetrahydroisoquinoline (1.0 molar equivalents) in ACN is added K₂CO₃ (VWR, 2.1 molar equivalents), followed by addition of 1-[bromo(4-fluorophenyl)methyl]-4-fluorobenzene (CombiBlocks, 1.05 molar equivalents). The reaction mixture is stirred overnight at 40° C. under N₂ atmosphere. Once the reaction reaches completion it is cooled to RT. Next, the crude reaction mixture is partitioned with H₂O. The phases are separated, and the organic phase is partitioned with brine. The organic layer is separated and concentrated under reduced pressure to afford the crude product. The crude product is purified by flash silica column chromatography to afford the title compound.

### Preparation of 5-fluoro-2-(1-(3-fluorobenzyl)piperidin-4-yl)-1,2,3,4-tetrahydroisoquinoline (Compound 91)

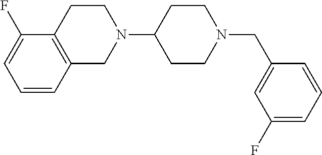 [SMILES: Fc1cccc(CN2CCC(N3CCc4c(F)cccc4C3)CC2)c1]

To a solution of 5-fluoro-2-(piperidin-4-yl)-1,2,3,4-tetrahydroisoquinoline (1.0 molar equivalents) in ACN is added K₂CO₃ (VWR, 2.1 molar equivalents), followed by addition of 1-(bromomethyl)-3-fluorobenzene (Aldrich, 1.05 molar equivalents). The reaction mixture is stirred overnight at 40° C. under N₂ atmosphere. Once the reaction reaches completion it is cooled to RT. Next, the crude reaction mixture is partitioned with H₂O. The phases are separated, and the organic phase is partitioned with brine. The organic layer is separated and concentrated under reduced pressure to afford the crude product. The crude product is purified by flash silica column chromatography to afford the title compound.

### Preparation of 2-(1-benzylpiperidin-4-yl)-1,2,3,4-tetrahydro-2,7-naphthyridine (Compound 92)

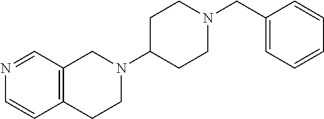 [SMILES: c1ccc(CN2CCC(N3CCc4ccncc4C3)CC2)cc1]

### Step a: Preparation of tert-butyl 4-(3,4-dihydro-2,7-naphthyridin-2(1H)-yl)piperidine-1-carboxylate

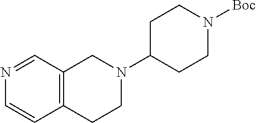 [SMILES: CC(C)(C)OC(=O)N1CCC(N2CCc3ccncc3C2)CC1]

To a mixture of 1-Boc-4-piperidinone (Chem Impex, 1.2 molar equivalents) and 1,2,3,4-tetrahydro-2,7-naphthyridine (Matrix Scientific, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Step b: Preparation of 2-(piperidin-4-yl)-1,2,3,4-tetrahydro-2,7-naphthyridine

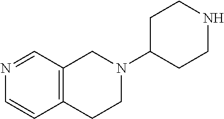 [SMILES: c1cc2c(cn1)CN(C1CCNCC1)CC2]

To a solution of tert-butyl 4-(3,4-dihydro-2,7-naphthyridin-2(1H)-yl)piperidine-1-carboxylate (1.0 molar equivalents) in DCM is added TFA (10.0 molar equivalents). The reaction mixture is stirred overnight at room temperature under N₂ atmosphere. Once the reaction mixture reaches completion the crude solution is concentrated under reduced pressure in the presence of toluene. The crude product is purified by flash silica column chromatography to afford the title compound.

### Step c: Preparation of 2-(1-benzylpiperidin-4-yl)-1,2,3,4-tetrahydro-2,7-naphthyridine (Compound 92)

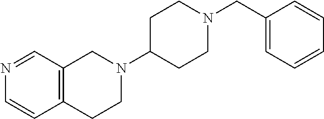 [SMILES: c1ccc(CN2CCC(N3CCc4ccncc4C3)CC2)cc1]

To a solution of 2-(piperidin-4-yl)-1,2,3,4-tetrahydro-2,7-naphthyridine (1.0 molar equivalents) in ACN is added K₂CO₃ (VWR, 2.1 molar equivalents), followed by addition of benzyl bromide (Aldrich, 1.05 molar equivalents). The reaction mixture is stirred overnight at 40° C. under N₂ atmosphere. Once the reaction reaches completion it is cooled to RT. Next, the crude reaction mixture is partitioned with H₂O.

The phases are separated, and the organic phase is partitioned with brine. The organic layer is separated and concentrated under reduced pressure to afford the crude product. The crude product is purified by flash silica column chromatography to afford the title compound.

### Preparation of 2-(1-(4-fluorobenzyl)piperidin-4-yl)-1,2,3,4-tetrahydro-2,7-naphthyridine (Compound 93)

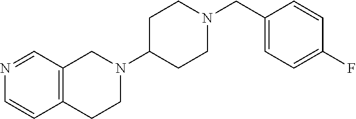 [SMILES: Fc1ccc(CN2CCC(N3CCc4ccncc4C3)CC2)cc1]

To a solution of 2-(piperidin-4-yl)-1,2,3,4-tetrahydro-2,7-naphthyridine (1.0 molar equivalents) in ACN is added K₂CO₃ (VWR, 2.1 molar equivalents), followed by addition of 1-(bromomethyl)-4-fluorobenzene (Aldrich, 1.05 molar equivalents). The reaction mixture is stirred overnight at 40° C. under N₂ atmosphere. Once the reaction reaches completion it is cooled to RT. Next, the crude reaction mixture is partitioned with H₂O. The phases are separated, and the organic phase is partitioned with brine. The organic layer is separated and concentrated under reduced pressure to afford the crude product. The crude product is purified by flash silica column chromatography to afford the title compound.

### Preparation of 2-(1-(bis(4-fluorophenyl)methyl)piperidin-4-yl)-1,2,3,4-tetrahydro-2,7-naphthyridine (Compound 94)

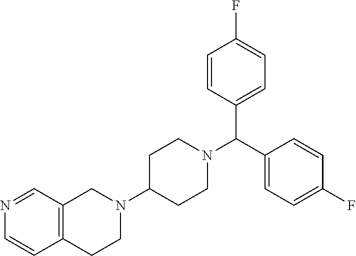 [SMILES: Fc1ccc(C(c2ccc(F)cc2)N2CCC(N3CCc4ccncc4C3)CC2)cc1]

To a solution of 2-(piperidin-4-yl)-1,2,3,4-tetrahydro-2,7-naphthyridine (1.0 molar equivalents) in ACN is added K₂CO₃ (VWR, 2.1 molar equivalents), followed by addition of 1-[bromo(4-fluorophenyl)methyl]-4-fluorobenzene (CombiBlocks, 1.05 molar equivalents). The reaction mixture is stirred overnight at 40° C. under N₂ atmosphere. Once the reaction reaches completion it is cooled to RT. Next, the crude reaction mixture is partitioned with H₂O. The phases are separated, and the organic phase is partitioned with brine. The organic layer is separated and concentrated under reduced pressure to afford the crude product. The crude product is purified by flash silica column chromatography to afford the title compound.

### Preparation of 2-(1-(3-fluorobenzyl)piperidin-4-yl)-1,2,3,4-tetrahydro-2,7-naphthyridine (Compound 95)

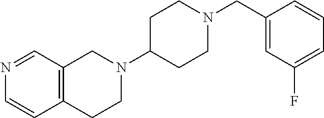 [SMILES: Fc1cccc(CN2CCC(N3CCc4ccncc4C3)CC2)c1]

To a solution of 2-(piperidin-4-yl)-1,2,3,4-tetrahydro-2,7-naphthyridine (1.0 molar equivalents) in ACN is added K₂CO₃ (VWR, 2.1 molar equivalents), followed by addition of 1-(bromomethyl)-3-fluorobenzene (Aldrich, 1.05 molar equivalents). The reaction mixture is stirred overnight at 40° C. under N₂ atmosphere. Once the reaction reaches completion it is cooled to RT. Next, the crude reaction mixture is partitioned with H₂O. The phases are separated, and the organic phase is partitioned with brine. The organic layer is separated and concentrated under reduced pressure to afford the crude product. The crude product is purified by flash silica column chromatography to afford the title compound.

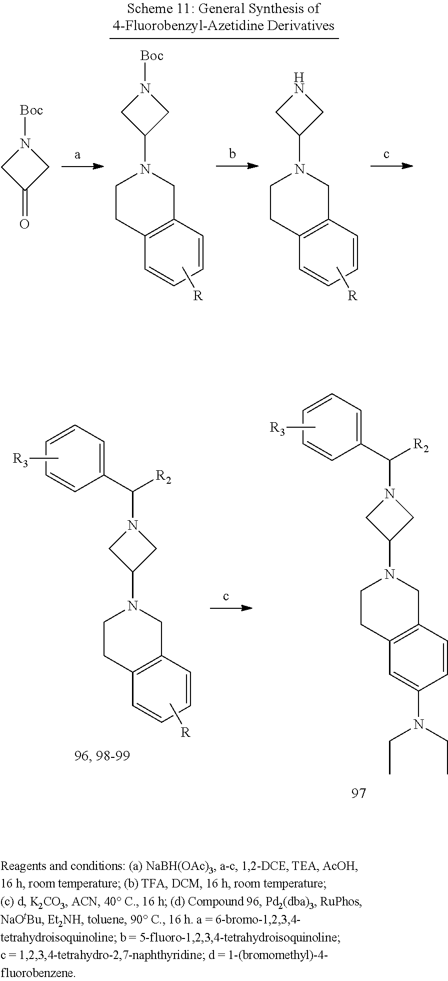

### Preparation of 6-bromo-2-(1-(4-fluorobenzyl)azetidin-3-yl)-1,2,3,4-tetrahydroisoquinoline (Compound 96)

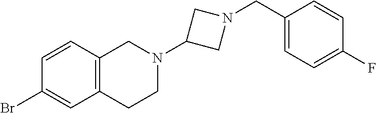 [SMILES: Fc1ccc(CN2CC(N3CCc4cc(Br)ccc4C3)C2)cc1]

### Step a: Preparation of tert-butyl 3-(6-bromo-3,4-dihydroisoquinolin-2(1H)-yl)azetidine-1-carboxylate

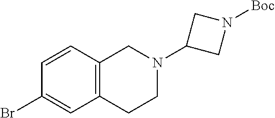 [SMILES: CC(C)(C)OC(=O)N1CC(N2CCc3cc(Br)ccc3C2)C1]

To a mixture of 1-Boc-3-azetidinone (Aldrich, 1.2 molar equivalents) and 6-bromo-1,2,3,4-tetrahydroisoquinoline (Matrix Scientific, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Step b: Preparation of 2-(azetidin-3-yl)-6-bromo-1,2,3,4-tetrahydroisoquinoline

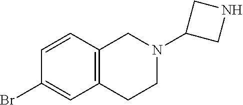 [SMILES: Brc1ccc2c(c1)CCN(C1CNC1)C2]

To a solution of tert-butyl 3-(6-bromo-3,4-dihydroisoquinolin-2(1H)-yl)azetidine-1-carboxylate (1.0 molar equivalents) in DCM is added TFA (10.0 molar equivalents). The reaction mixture is stirred overnight at room temperature under N₂ atmosphere. Once the reaction mixture reaches completion the crude solution is concentrated under reduced pressure in the presence of toluene. The crude product is purified by flash silica column chromatography to afford the title compound.

### Step c: Preparation of 6-bromo-2-(1-(4-fluorobenzyl)azetidin-3-yl)-1,2,3,4-tetrahydroisoquinoline (Compound 96)

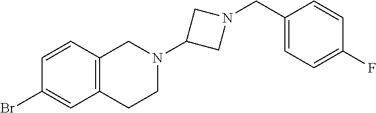 [SMILES: Fc1ccc(CN2CC(N3CCc4cc(Br)ccc4C3)C2)cc1]

To a solution of 2-(azetidin-3-yl)-6-bromo-1,2,3,4-tetrahydroisoquinoline (1.0 molar equivalents) in ACN is added K₂CO₃ (VWR, 2.1 molar equivalents), followed by addition of 1-(bromomethyl)-4-fluorobenzene (Aldrich, 1.05 molar equivalents). The reaction mixture is stirred overnight at 40° C. under N₂ atmosphere. Once the reaction reaches completion it is cooled to RT. Next, the crude reaction mixture is partitioned with H₂O. The phases are separated, and the organic phase is partitioned with brine. The organic layer is separated and concentrated under reduced pressure to afford the crude product. The crude product is purified by flash silica column chromatography to afford the title compound.

Preparation of N,N-diethyl-2-(1-(4-fluorobenzyl)azetidin-3-yl)-1,2,3,4-tetrahydroisoquinolin-6-amine (Compound 97)

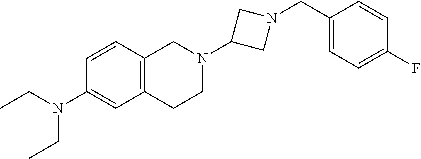 [SMILES: CCN(CC)c1ccc2c(c1)CCN(C1CN(Cc3ccc(F)cc3)C1)C2]

To a solution of Compound 96 (1.0 molar equivalents) in anhydrous toluene is added a pre-dissolved solution containing Pd₂(dba)₃ (Strem, 0.10 molar equivalents), and RuPhos (CombiBlocks, 0.20 molar equivalents) in toluene. Next, diethylamine (Alfa Aesar, 15 molar equivalents) is added, followed by NaO^{t}Bu (AK Scientific, 1.5 molar equivalents). The reaction mixture is stirred overnight at 90° C. under N₂ atmosphere. Once the reaction reaches completion it is cooled to RT and the reaction mixture is partitioned between ethyl acetate and H₂O. The phases are separated, and the organic phase is partitioned a second time with H₂O, followed by brine. The organic layer is separated and dried over anhydrous sodium sulfate, filtered and concentrated under reduced pressure to afford the crude product. The crude product is purified by flash silica column chromatography to afford the title compound.

### Preparation of 5-fluoro-2-(1-(4-fluorobenzyl)azetidin-3-yl)-1,2,3,4-tetrahydroisoquinoline (Compound 98)

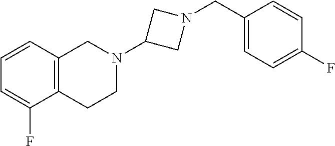 [SMILES: Fc1ccc(CN2CC(N3CCc4c(F)cccc4C3)C2)cc1]

### Step a: Preparation of tert-butyl 3-(5-fluoro-3,4-dihydroisoquinolin-2(1H)-yl)azetidine-1-carboxylate

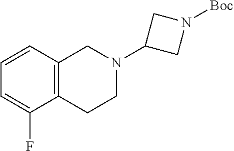 [SMILES: CC(C)(C)OC(=O)N1CC(N2CCc3c(F)cccc3C2)C1]

To a mixture of 1-Boc-3-azetidinone (Aldrich, 1.2 molar equivalents) and 5-fluoro-1 2,3,4-tetrahydroisoquinoline (Combi-Blocks, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Step b: Preparation of 2-(azetidin-3-yl)-5-fluoro-1,2,3,4-tetrahydroisoquinoline

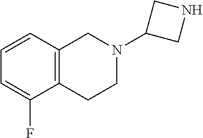 [SMILES: Fc1cccc2c1CCN(C1CNC1)C2]

To a solution of tert-butyl 3-(5-fluoro-3,4-dihydroisoquinolin-2(1H)-yl)azetidine-1-carboxylate (1.0 molar equivalents) in DCM is added TFA (10.0 molar equivalents). The reaction mixture is stirred overnight at room temperature under N₂ atmosphere. Once the reaction mixture reaches completion the crude solution is concentrated under reduced pressure in the presence of toluene. The crude product is purified by flash silica column chromatography to afford the title compound.

### Step c: Preparation of 5-fluoro-2-(1-(4-fluorobenzyl)azetidin-3-yl)-1,2,3,4-tetrahydroisoquinoline (Compound 98)

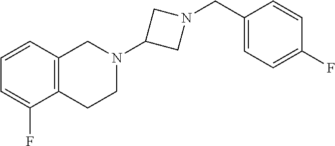 [SMILES: Fc1ccc(CN2CC(N3CCc4c(F)cccc4C3)C2)cc1]

To a solution of 2-(azetidin-3-yl)-5-fluoro-1,2,3,4-tetrahydroisoquinoline (1.0 molar equivalents) in ACN is added K₂CO₃ (VWR, 2.1 molar equivalents), followed by addition of 1-(bromomethyl)-4-fluorobenzene (Aldrich, 1.05 molar equivalents). The reaction mixture is stirred overnight at 40° C. under N₂ atmosphere. Once the reaction reaches completion it is cooled to RT. Next, the crude reaction mixture is partitioned with H₂O. The phases are separated, and the organic phase is partitioned with brine. The organic layer is separated and concentrated under reduced pressure to afford the crude product. The crude product is purified by flash silica column chromatography to afford the title compound.

### Preparation of 2-(1-(4-fluorobenzyl)azetidin-3-yl)-1,2,3,4-tetrahydro-2,7-naphthyridine (Compound 99)

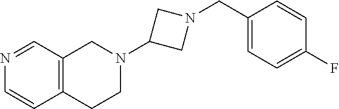 [SMILES: Fc1ccc(CN2CC(N3CCc4ccncc4C3)C2)cc1]

### Step a: Preparation of tert-butyl 3-(3,4-dihydro-2,7-naphthyridin-2(1H)-yl)azetidine-1-carboxylate

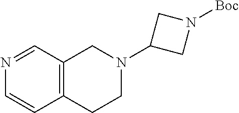 [SMILES: CC(C)(C)OC(=O)N1CC(N2CCc3ccncc3C2)C1]

To a mixture of 1-Boc-3-azetidinone (Aldrich, 1.2 molar equivalents) and 1,2,3,4-tetrahydro-2,7-naphthyridine (Combi-Blocks, 1.0 molar equivalents) in 1,2-dichloroethane is added triethylamine (0.75 molar equivalents). The reaction mixture is stirred at room temperature for 30 minutes. Acetic acid (1.75 molar equivalents) is then added and the solution is stirred for another 30 minutes. Next, sodium triacetoxyborohydride (Arctom Chemicals, 1.5 molar equivalents) is added and the reaction mixture is stirred overnight at room temperature under N₂ atmosphere. Upon completion, the reaction mixture is quenched with saturated sodium bicarbonate solution. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

### Step b: Preparation of 2-(azetidin-3-yl)-1,2,3,4-tetrahydro-2,7-naphthyridine

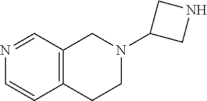 [SMILES: c1cc2c(cn1)CN(C1CNC1)CC2]

To a solution of tert-butyl 3-(3,4-dihydro-2,7-naphthyridin-2(1H)-yl)azetidine-1-carboxylate (1.0 molar equivalents) in DCM is added TFA (10.0 molar equivalents). The reaction mixture is stirred overnight at room temperature under N₂ atmosphere. Once the reaction mixture reaches completion the crude solution is concentrated under reduced pressure in the presence of toluene. The crude product is purified by flash silica column chromatography to afford the title compound.

### Step c: Preparation of 2-(1-(4-fluorobenzyl)azetidin-3-yl)-1,2,3,4-tetrahydro-2,7-naphthyridine (Compound 99)

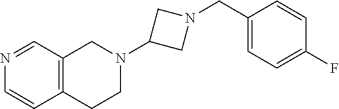 [SMILES: Fc1ccc(CN2CC(N3CCc4ccncc4C3)C2)cc1]

To a solution of 2-(azetidin-3-yl)-1,2,3,4-tetrahydro-2,7-naphthyridine (1.0 molar equivalents) in ACN is added K₂CO₃ (VWR, 2.1 molar equivalents), followed by addition of 1-(bromomethyl)-4-fluorobenzene (Aldrich, 1.05 molar equivalents). The reaction mixture is stirred overnight at 40° C. under N₂ atmosphere. Once the reaction reaches completion it is cooled to RT. Next, the crude reaction mixture is partitioned with H₂O. The phases are separated, and the organic phase is partitioned with brine. The organic layer is separated and concentrated under reduced pressure to afford the crude product. The crude product is purified by flash silica column chromatography to afford the title compound.

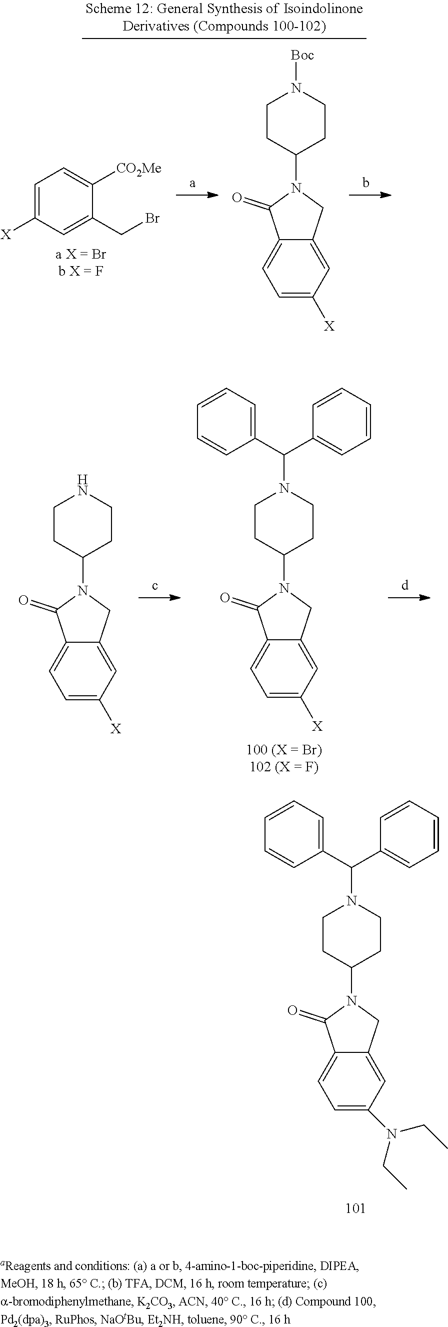

### Preparation of 2-(1-benzhydrylpiperidin-4-yl)-5-bromoisoindolin-1-one (Compound 100)

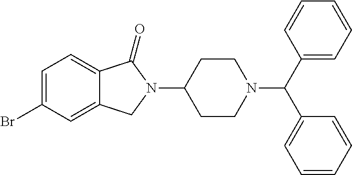 [SMILES: O=C1c2ccc(Br)cc2CN1C1CCN(C(c2ccccc2)c2ccccc2)CC1]

### Step a: Preparation of tert-butyl 4-(5-bromo-1-oxoisoindolin-2-yl)piperidine-1-carboxylate

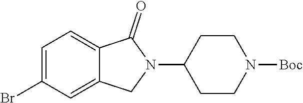 [SMILES: CC(C)(C)OC(=O)N1CCC(N2Cc3cc(Br)ccc3C2=O)CC1]

A mixture of methyl 4-bromo-2-(bromomethyl)benzoate (Aldrich, 1.0 molar equivalents) mL), 4-amino-1-Boc-piperidine (Aldrich, 1.5 molar equivalents) and diisopropylamine (Aldrich, 2.5 molar equivalents) in methanol is heated overnight at 65° C. while under N₂ atmosphere. Once the reaction reaches completion, it is cooled to RT. Next, the crude reaction mixture is partitioned between DCM and 1 N HCl. The phases are separated, and the organic phase is partitioned with brine. The organic layer is separated and concentrated under reduced pressure to afford the crude product. The crude product is purified by flash silica column chromatography to afford the title compound.

### Step b: Preparation of 5-bromo-2-(piperidin-4-yl)isoindolin-1-one

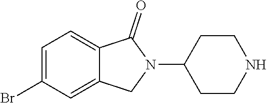 [SMILES: O=C1c2ccc(Br)cc2CN1C1CCNCC1]

To a solution of tert-butyl 4-(5-bromo-1-oxoisoindolin-2-yl)piperidine-1-carboxylate (1.0 molar equivalents) in DCM is added TFA (10.0 molar equivalents). The reaction mixture is stirred overnight at room temperature under N₂ atmosphere. Once the reaction mixture reaches completion the crude solution is concentrated under reduced pressure in the presence of toluene. The crude product is purified by flash silica column chromatography to afford the title compound.

### Step c: Preparation of 2-(1-benzhydrylpiperidin-4-yl)-5-bromoisoindolin-1-one (Compound 100)

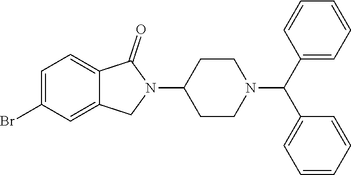 [SMILES: O=C1c2ccc(Br)cc2CN1C1CCN(C(c2ccccc2)c2ccccc2)CC1]

To a solution of 5-bromo-2-(piperidin-4-yl)isoindolin-1-one (1.0 molar equivalents) in ACN is added K₂CO₃ (VWR, 2.1 molar equivalents), followed by addition of α-bromodiphenylmethane (TCl, 1.05 molar equivalents). The reaction mixture is stirred overnight at 40° C. under N₂ atmosphere. Once the reaction reaches completion it is cooled to RT. Next, the crude reaction mixture is partitioned with H₂O. The phases are separated, and the organic phase is partitioned with brine. The organic layer is separated and concentrated under reduced pressure to afford the crude product. The crude product is purified by flash silica column chromatography to afford the title compound.

### Preparation of 2-(1-benzhydrylpiperidin-4-yl)-5-(diethylamino)isoindolin-1-one (Compound 101)

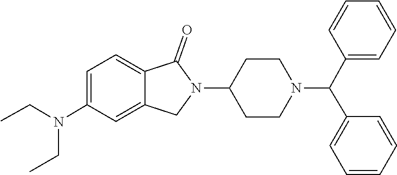 [SMILES: CCN(CC)c1ccc2c(c1)CN(C1CCN(C(c3ccccc3)c3ccccc3)CC1)C2=O]

To a solution of Compound 100 (1.0 molar equivalents) in anhydrous toluene is added a pre-dissolved solution containing Pd₂(dba)₃ (Strem, 0.10 molar equivalents), and RuPhos (CombiBlocks, 0.20 molar equivalents) in toluene. Next, diethylamine (Alfa Aesar, 15 molar equivalents) is added, followed by NaO^{t}Bu (AK Scientific, 1.5 molar equivalents). The reaction mixture is stirred overnight at 90° C. under N₂ atmosphere. Once the reaction reaches completion it is cooled to RT and the reaction mixture is partitioned between ethyl acetate and H₂O. The phases are separated, and the organic phase is partitioned a second time with H₂O, followed by brine. The organic layer is separated and dried over anhydrous sodium sulfate, filtered and concentrated under reduced pressure to afford the crude product. The crude product is purified by flash silica column chromatography to afford the title compound.

### Preparation of 2-(1-benzhydrylpiperidin-4-yl)-5-fluoroisoindolin-1-one (Compound 102)

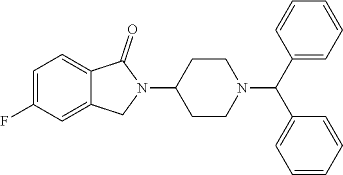 [SMILES: O=C1c2ccc(F)cc2CN1C1CCN(C(c2ccccc2)c2ccccc2)CC1]

### Step a: Preparation of tert-butyl 4-(5-fluoro-1-oxoisoindolin-2-yl)piperidine-1-carboxylate

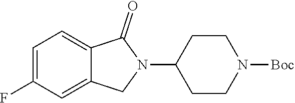 [SMILES: CC(C)(C)OC(=O)N1CCC(N2Cc3cc(F)ccc3C2=O)CC1]

A mixture of methyl 2-(bromomethyl)-4-fluorobenzoate (Enamine, 1.0 molar equivalents) mL), 4-amino-1-boc-piperidine (Aldrich, 1.5 molar equivalents) and diisopropylamine (Aldrich, 2.5 molar equivalents) in methanol is heated overnight at 65° C. while under N₂ atmosphere. Once the reaction reaches completion, it is cooled to RT. Next, the crude reaction mixture is partitioned between DCM and 1 N HCl. The phases are separated, and the organic phase is partitioned with brine. The organic layer is separated and concentrated under reduced pressure to afford the crude product. The crude product is purified by flash silica column chromatography to afford the title compound.

### Step b: Preparation of 5-fluoro-2-(piperidin-4-yl)isoindolin-1-one

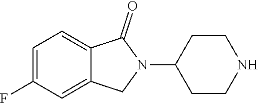 [SMILES: O=C1c2ccc(F)cc2CN1C1CCNCC1]

To a solution of tert-butyl 4-(5-fluoro-1-oxoisoindolin-2-yl)piperidine-1-carboxylate (1.0 molar equivalents) in DCM is added TFA (10.0 molar equivalents). The reaction mixture is stirred overnight at room temperature under N₂ atmosphere. Once the reaction mixture reaches completion the crude solution is concentrated under reduced pressure in the presence of toluene. The crude product is purified by flash silica column chromatography to afford the title compound.

### Step c: Preparation of 2-(1-benzhydrylpiperidin-4-yl)-5-bromoisoindolin-1-one (Compound 102)

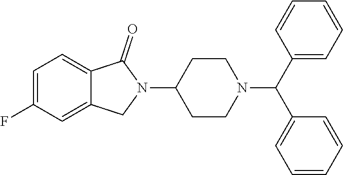 [SMILES: O=C1c2ccc(F)cc2CN1C1CCN(C(c2ccccc2)c2ccccc2)CC1]

To a solution of 5-fluoro-2-(piperidin-4-yl)isoindolin-1-one (1.0 molar equivalents) in ACN is added K₂CO₃ (VWR, 2.1 molar equivalents), followed by addition of α-bromodiphenylmethane (TCl, 1.05 molar equivalents). The reaction mixture is stirred overnight at 40° C. under N₂ atmosphere. Once the reaction reaches completion it is cooled to RT. Next, the crude reaction mixture is partitioned with H₂O. The phases are separated, and the organic phase is partitioned with brine. The organic layer is separated and concentrated under reduced pressure to afford the crude product. The crude product is purified by flash silica column chromatography to afford the title compound.

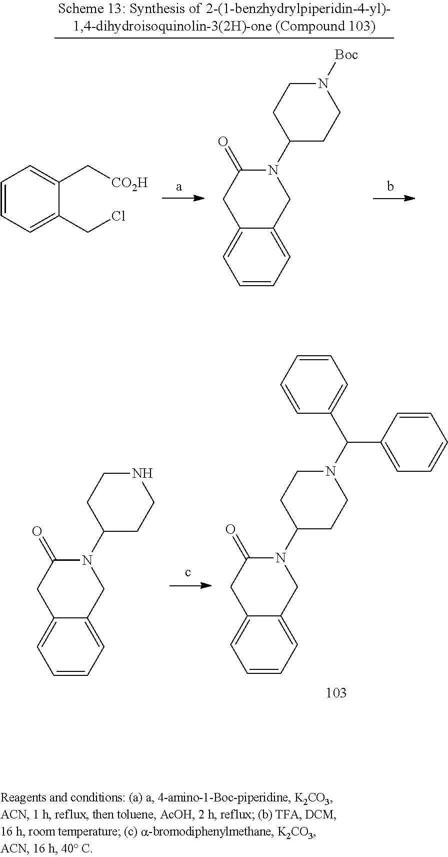

### Step a: Preparation of tert-butyl 4-(3-oxo-3,4-dihydroisoquinolin-2(1H)-yl)piperidine-1-carboxylate

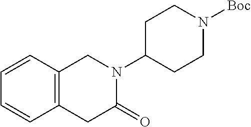 [SMILES: CC(C)(C)OC(=O)N1CCC(N2Cc3ccccc3CC2=O)CC1]

Potassium carbonate (Aldrich, 2.1 molar equivalents) is added to a solution of 2-(2-(chloromethyl)phenyl)acetic acid (Aldrich, 1.0 molar equivalents) in ACN. Next, 4-amino-1-Boc-piperidine (Aldrich, 1.20 molar equivalents) is added and the reaction mixture is heated to reflux for 1 hour under N₂ atmosphere. After the reaction reaches completion it is cooled to room temperature and then filtered over a bed of celite. The filtrate is concentrated under reduced pressure. Next, the crude reaction residue is diluted with toluene and acetic acid (5.5 molar equivalents) is added. The reaction mixture is heated to reflux for 2 hours. After the reaction reaches completion it is cooled to room temperature and then concentrated under reduced pressure. The residue is treated with ethyl acetate. The mixture is washed successively with aqueous sodium bicarbonate, 1 N HCl, and brine. The organic layer is dried over magnesium sulfate, filtered, and concentrated under reduced pressure. The crude product is purified by flash silica column chromatography to afford the title compound.

### Step b: Preparation of 2-(piperidin-4-yl)-1,4-dihydroisoquinolin-3(2H)-one

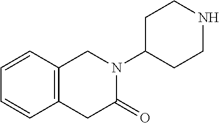 [SMILES: O=C1Cc2ccccc2CN1C1CCNCC1]

To a solution of tert-butyl 4-(3-oxo-3,4-dihydroisoquinolin-2(1H)-yl)piperidine-1-carboxylate (1.0 molar equivalents) in DCM is added TFA (10.0 molar equivalents). The reaction mixture is stirred overnight at room temperature under N₂ atmosphere. Once the reaction mixture reaches completion the crude solution is concentrated under reduced pressure in the presence of toluene. The crude product is purified by flash silica column chromatography to afford the title compound.

### Step c: Preparation of 2-(1-benzhydrylpiperidin-4-yl)-1,4-dihydroisoquinolin-3(2H)-one (Compound 103)

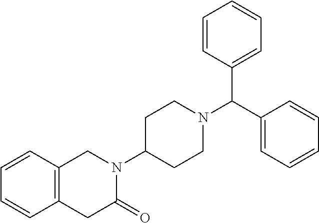 [SMILES: O=C1Cc2ccccc2CN1C1CCN(C(c2ccccc2)c2ccccc2)CC1]

To a solution of 2-(piperidin-4-yl)-1,4-dihydroisoquinolin-3(2H)-one (1.0 molar equivalents) in ACN is added K₂CO₃ (VWR, 2.05 molar equivalents), followed by addition of α-bromodiphenylmethane (TCl, 1.05 molar equivalents). The reaction mixture is stirred overnight at 40° C. under N₂ atmosphere. Once the reaction reaches completion it is cooled to RT. Next, the crude reaction mixture is partitioned with H₂O. The phases are separated, and the organic phase is partitioned with brine. The organic layer is separated and concentrated under reduced pressure to afford the crude product. The crude product is purified by flash silica column chromatography to afford the title compound.

### Preparation of 2-(1-benzhydrylpiperidin-4-yl)-3,4-dihydroisoquinolin-1 (2H)-one (Compound 104)

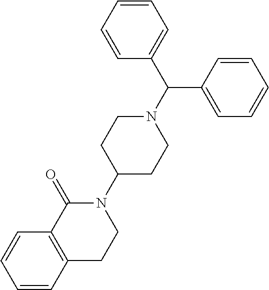 [SMILES: O=C1c2ccccc2CCN1C1CCN(C(c2ccccc2)c2ccccc2)CC1]

Compound 104 is prepared from a procedure analogous to that for Compound 103 as described immediately above. To a solution of 2-(piperidin-4-yl)-3,4-dihydroisoquinolin-1(2H)-one (1.0 molar equivalents) in ACN is added K₂CO₃ (VWR, 2.05 molar equivalents), followed by addition of α-bromodiphenylmethane (TCl, 1.05 molar equivalents). The reaction mixture is stirred overnight at 40° C. under N₂ atmosphere. Once the reaction reaches completion it is cooled to RT. Next, the crude reaction mixture is partitioned with H₂O. The phases are separated, and the organic phase is partitioned with brine. The organic layer is separated and concentrated under reduced pressure to afford the crude product. The crude product is purified by flash silica column chromatography to afford the title compound.

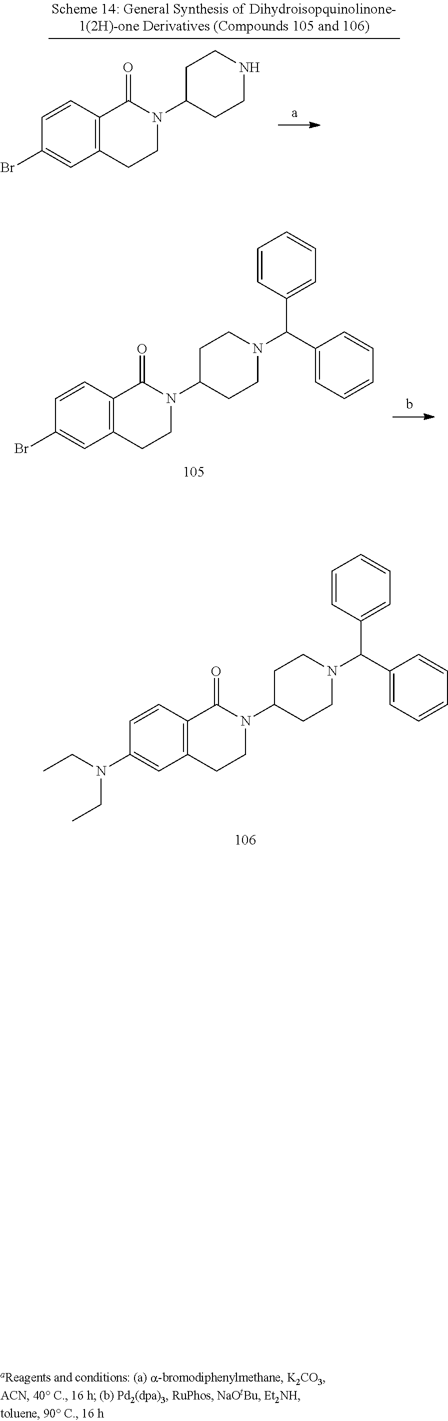 [SMILES: O=C1c2ccc(Br)cc2CCN1C1CCNCC1 ; O=C1c2ccc(Br)cc2CCN1C1CCN(C(c2ccccc2)c2ccccc2)CC1 ; CCN(CC)c1ccc2c(c1)CCN(C1CCN(C(c3ccccc3)c3ccccc3)CC1)C2=O]

### Preparation of 2-(1-benzhydrylpiperidin-4-yl)-6-bromo-3,4-dihydroisoquinolin-1(2H)-one (Compound 105)

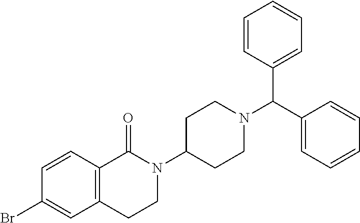 [SMILES: O=C1c2ccc(Br)cc2CCN1C1CCN(C(c2ccccc2)c2ccccc2)CC1]

To a solution of 6-bromo-2-(piperidin-4-yl)-3,4-dihydroisoquinolin-1(2H)-one (Aurora Fine Chemicals, 1.0 molar equivalents) in ACN is added K₂CO₃ (VWR, 2.05 molar equivalents), followed by addition of α-bromodiphenylmethane (TCl, 1.05 molar equivalents). The reaction mixture is stirred overnight at 40° C. under N₂ atmosphere. Once the reaction reaches completion it is cooled to RT. Next, the crude reaction mixture is partitioned with H₂O. The phases are separated, and the organic phase is partitioned with brine. The organic layer is separated and concentrated under reduced pressure to afford the crude product. The crude product is purified by flash silica column chromatography to afford the title compound.

### Preparation of 2-(1-benzhydrylpiperidin-4-yl)-6-(diethylamino)-3,4-dihydroisoquinolin-1(2H)-one (Compound 106)

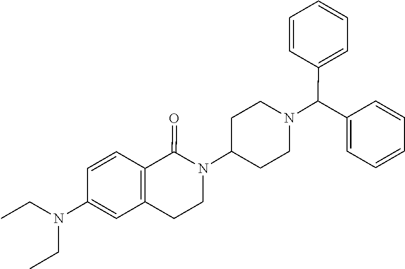 [SMILES: CCN(CC)c1ccc2c(c1)CCN(C1CCN(C(c3ccccc3)c3ccccc3)CC1)C2=O]

To a solution of Compound 104 (1.0 molar equivalents) in anhydrous toluene is added a pre-dissolved solution containing Pd₂(dba)₃ (Strem, 0.10 molar equivalents), and RuPhos (CombiBlocks, 0.20 molar equivalents) in toluene (10 mL). Next, diethylamine (Alfa Aesar, 15 molar equivalents) is added, followed by NaO^{t}Bu (AK Scientific, 1.5 molar equivalents). The reaction mixture is stirred overnight at 90° C. under N₂ atmosphere. Once the reaction reaches completion it is cooled to RT and the reaction mixture is partitioned between ethyl acetate and H₂O. The phases are separated, and the organic phase is partitioned a second time with H₂O, followed by brine. The organic layer is separated and dried over anhydrous sodium sulfate, filtered and concentrated under reduced pressure to afford the crude product. The crude product is purified by flash silica column chromatography to afford the title compound.

### Example 2. Thermal Shift Assay (TSA)

TSA was utilized to biophysically characterize the recombinant human FGFR1/FGF2 complex in the presence or absence of compounds. The assay functions by protein denaturation over a temperature gradient. During protein unfolding, exposed hydrophobic regions bind a dye and fluoresce due to solvent relaxation effects. Changes in the melting temperature of the protein complex in the presence of each compound were monitored and compounds were screened/ranked using this method.

### FGFR1 Protein Expression and Purification

One Shot BL21 (DE3) Star Escherichia coli competent cells (Thermo Fisher) were transformed with the relevant FGFR1 plasmid and inoculated onto Ampicillin Luria Broth/Agar plates. Two hundred milliliter portions of Terrific Broth starter cultures were used to inoculate 9 L cultures with ampicillin at a concentration of 100 μg/mL. Cultures were grown to an O.D.600 near 1.0 at 37° C. and induced with isopropyl β-D-1-thiogalactopyranoside (IPTG) for 5 hours at 37° C. The cells were then harvested by centrifugation using a F9-6×1000 LEX rotor at 6000 rpm for 10 min at 4° C. in a Sorvall Lynx 6000 centrifuge (Thermo Scientific). Bacterial pellets were stored at −80° C. until use.

Cell pellets were thawed and resuspend in 100 mL of FGFR1 Lysis Buffer per 9 g of pellet (20 mM Tris-HCl pH 8.0, 500 mM NaCl, 1 mM dithiothreitol) by stirring at 4° C. for 1 hour. Cells were lysed in 3 cycles on/off for 3 minutes each at 4° C. via sonication followed by centrifugation for 30 minutes at 16,000 RPM in rotor F20 at 4° C., after which the supernatant was discarded. This process was then repeated twice. The pellets were resuspended in 150 mL FGFR1 solubilization buffer (8 M urea, 20 mM Tris-HCL pH 8.0, 150 mM NaCl, 1 mM dithiothreitol) by stirring for 1 hour at 4° C., and the solution was subjected to centrifugation for 30 minutes at 16,000 RPM in rotor F20 at 4° C. The pellets were discarded, and the supernatant was filtered through a 0.45 μM polyethersylfone (PES) filter. After filtration, the supernatant was added dropwise to 1 L FGFR1 refolding buffer (20 mM Tris-HCl pH 8.0, 150 mM NaCl, 0.5 M L-arginine, 25 mM MgCl₂) using a glass column. Protein was concentrated by tangential flow from 1 L to 100 mL and dialyzed against 1 L of FGFR1 Dialysis Buffer (20 mM Tris-HCl pH 8.0, 150 mM NaCl, 25 mM MgCl₂) for 2 hours at 4° C., and the dialysis step was repeated with fresh buffer for an additional 2 hours at 4° C. The material thus obtained was then centrifuged at 4000 RPM in Eppendorf tabletop centrifuge for 5 minutes and loaded onto 2×5 mL heparin columns. The columns were washed extensively (20 CV) using FGFR1 Heparin Buffer A (20 mM Tris-HCl pH 8.0, 150 mM NaCl, 25 mM MgCl₂) and then eluted using FGFR1 Heparin Buffer B (20 mM Tris-HCl pH 8.0, 1.5 M NaCl, 25 mM MgCl₂). A large peak was recovered that was >95% pure by SDS-PAGE analysis gel (Expected Mw: 25 KDa). The protein was collected and diluted in 20 mM Tris-HCl pH 8.0, 25 mM MgCl₂ buffer in order to reach a NaCl concentration of 150 mM. The FGFR1 thus obtained was concentrated and stored at −80° C.

### FGF2 Protein Expression and Purification

One Shot BL21 (DE3) Star Escherichia coli competent cells (Thermo Fisher) were transformed with a relevant FGF2 plasmid and inoculated onto Ampicillin Luria Broth/Agar plates. Two hundred milliliter portions of Terrific Broth starter cultures were used to inoculate 9 L cultures with ampicillin at a concentration of 100 μg/mL. Cultures were grown to an O.D.600 near 1.0 at 37° C., and induced with IPTG overnight at 18° C. The cells were harvested at 7000 RPM in rotor 6000 for 5 min at 4° C. and stored at −80° C. Bacterial pellets were resuspended in 25 mM Hepes-NaOH, pH 7.5, 250 mM NaCl, and the cells were lysed in 3 cycles on/off for 3 minutes each at 4° C. via sonication. After centrifugation for 30 minutes at 16,000 RPM at 4° C., the isolated pellets were discarded, and the supernatant was filtered supernatant through a 0.45 μM PES filter using 100 mL superloop. The lysate was purified over a 5 mL S column by washing the column with Lysis buffer for 5 CV then eluting using gradient from 250 mM to 1 M NaCl over 20 CV. The fractions containing FGF2 were identified via SDS-PAGE gel (Expected Mw: 15.2 KDa). The protein was collected and diluted in 20 mM Tris-HCl pH 8.0, 25 mM MgCl₂ buffer in order to reach a NaCl concentration of 150 mM. The purified FGF2 was concentrated and stored at −80° C.

### FGFR1/FGF-2 Complex Formation and TSA Protocol

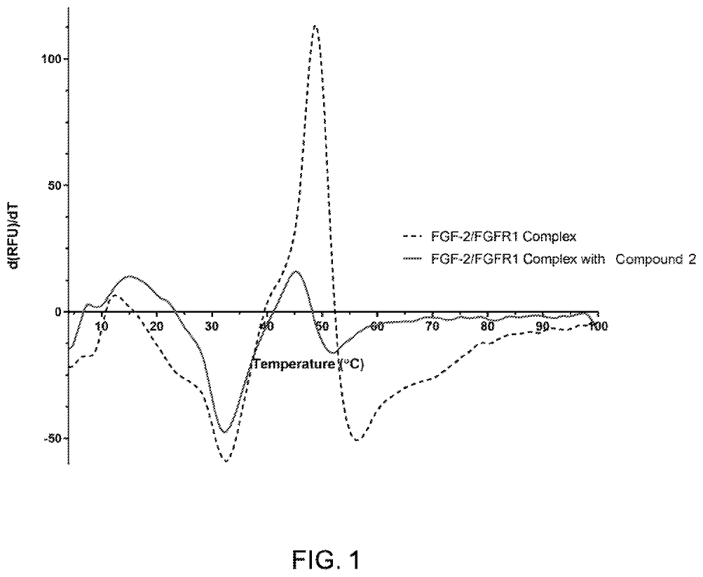

Thawed aliquots of purified FGF2 (1.0 mg/mL) and FGFR1 (1.6 mg/mL) proteins were mixed in a 1:1 molar ratio (64 μM: 64 μM) on ice for 30 min at 4° C. and plated prior to the thermal shift assay (TSA). Complex formation was verified by loading the complexed material on a size exclusion column (superdex 300GL S200) and observing the monodisperse peak corresponding to the FGF2/FGFR1 complex (˜40 kDa). Compounds of the invention were screened in dose response format (0-100 μM) with the FGF2/FGFR1 complex in triplicates. FGF2/FGFR1/compound complexes were mixed in a 1000:1 ratio with Sypro Orange dye (Sigma-Aldrich). The samples were processed using a Bio-Rad CFX C₉₆ Touch quantitative polymerase chain reaction and run using the FRET assay settings with a heating ramp of 0.3° C./s cycling from 4 to 100° C. Data analysis was performed using the Bio-Rad CFX Manager Software (version 3.1, Bio-Rad) and changes in the melting temperature (Tm) of the complex in the presence of each compound were monitored. The results are shown in Tables 2 and 3 below and also in FIG. 1 (for Compound 2).

FIG. 1 shows a thermal stability assay (TSA) of the purified FGF-2/FGFR1 complex with and without Compound 2. The curve of the complex alone (dotted line) shows two positive peaks, one corresponding to FGF-2 (left) and one to FGFR1 (right). In the presence of 10 μM Compound 2 (solid line), the TSA shows a shift of the melting curve, in effect moving the peaks closer together. This indicates binding of Compound 2 and increased stability of the complex.

TABLE 2
TSA Results for Compounds 1 and 2

| | | ΔTm (° C.)- | ΔTm (° C.)- |
|---|---|---|---|
| Compound | Structure | FGFR1 | FGF2 |
| 1 | 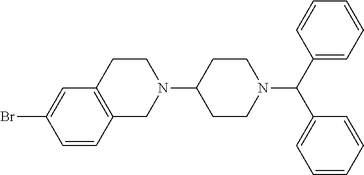 [SMILES: Brc1ccc2c(c1)CCN(C1CCN(C(c3ccccc3)c3ccccc3)CC1)C2] | +2.0 (10 μM) +3.0 (100 μM) | −1.5 (10 μM) −1.8 (100 μM) |
| 2 | 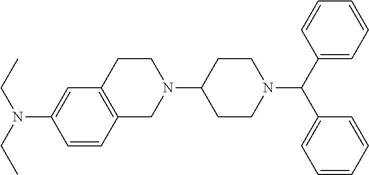 [SMILES: CCN(CC)c1ccc2c(c1)CCN(C1CCN(C(c3ccccc3)c3ccccc3)CC1)C2] | +4.0 (2 μM) +4.5 (10 μM) | −2.5 (2 μM) −3.0 (10 μM) |

TABLE 3
TSA Results for Selected Compounds of this Invention

| | | ΔTm (° C.)- | ΔTm (° C.)- |
|---|---|---|---|
| Compound | Structure | FGFR1 | FGF2 |
| 3 | 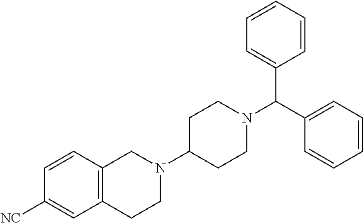 [SMILES: N#Cc1ccc2c(c1)CCN(C1CCN(C(c3ccccc3)c3ccccc3)CC1)C2] | 0 (25 μM) | +3.7 (25 μM) |
| 4 | 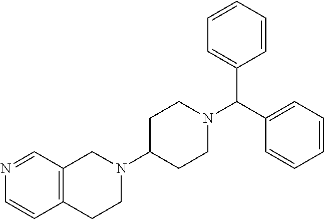 [SMILES: c1ccc(C(c2ccccc2)N2CCC(N3CCc4ccncc4C3)CC2)cc1] | +14.5 (2 μM) | −2.2 (2 μM) |
| 5 | 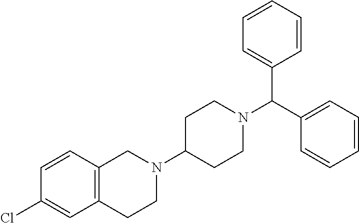 [SMILES: Clc1ccc2c(c1)CCN(C1CCN(C(c3ccccc3)c3ccccc3)CC1)C2] | +1.2 (25 μM) | −1.7 (25 μM) |
| 6 | 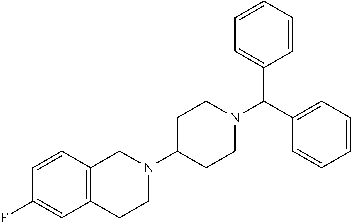 [SMILES: Fc1ccc2c(c1)CCN(C1CCN(C(c3ccccc3)c3ccccc3)CC1)C2] | +1.5 (25 μM) | 0 (25 μM) |
| 7 | 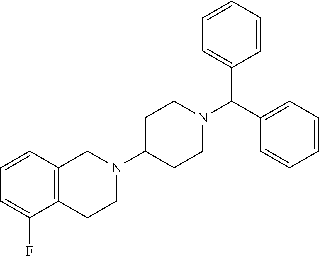 [SMILES: Fc1cccc2c1CCN(C1CCN(C(c3ccccc3)c3ccccc3)CC1)C2] | +10.7 (10 μM) | −0.2 (10 μM) |
| 8 | 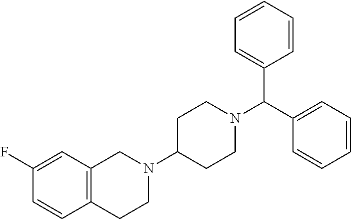 [SMILES: Fc1ccc2c(c1)CN(C1CCN(C(c3ccccc3)c3ccccc3)CC1)CC2] | +3.3 (10 μM) | −0.5 (10 μM) |
| 9 | 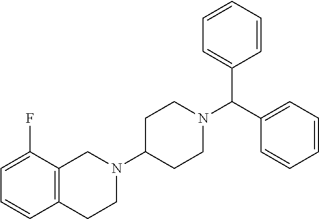 [SMILES: Fc1cccc2c1CN(C1CCN(C(c3ccccc3)c3ccccc3)CC1)CC2] | 0 (100 μM) | 0 (100 μM) |
| 14 | 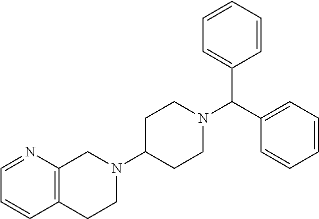 [SMILES: c1ccc(C(c2ccccc2)N2CCC(N3CCc4cccnc4C3)CC2)cc1] | +5.4 (25 μM) | −3.3 (25 μM) |
| 17 | 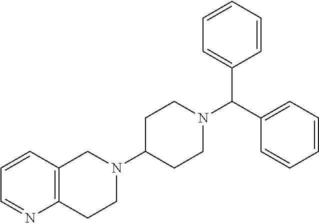 [SMILES: c1ccc(C(c2ccccc2)N2CCC(N3CCc4ncccc4C3)CC2)cc1] | +3.0 (10 μM) | −2.7 (10 μM) |
| 19 | 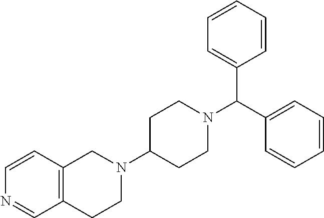 [SMILES: c1ccc(C(c2ccccc2)N2CCC(N3CCc4cnccc4C3)CC2)cc1] | +2.8 (25 μM) | −3.0 (25 μM) |
| 21 | 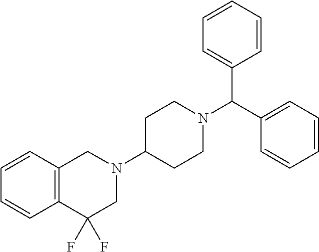 [SMILES: FC1(F)CN(C2CCN(C(c3ccccc3)c3ccccc3)CC2)Cc2ccccc21] | +11.6 (2 μM) | −3.6 (2 μM) |
| 38 | 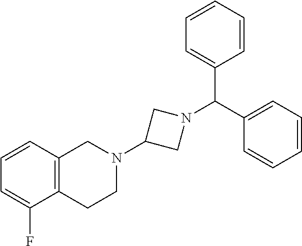 [SMILES: Fc1cccc2c1CCN(C1CN(C(c3ccccc3)c3ccccc3)C1)C2] | +9.8 (2 μM) | −2.7 (2 μM) |
| 43 | 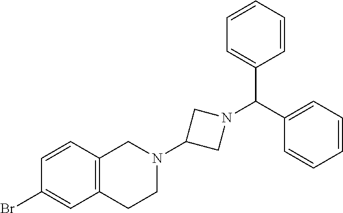 [SMILES: Brc1ccc2c(c1)CCN(C1CN(C(c3ccccc3)c3ccccc3)C1)C2] | +6.6 (100 μM) | −4.5 (100 μM) |
| 56 | 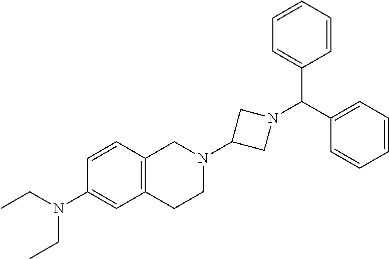 [SMILES: CCN(CC)c1ccc2c(c1)CCN(C1CN(C(c3ccccc3)c3ccccc3)C1)C2] | −0.5 (100 μM) | 0 (100 μM) |
| 81 | 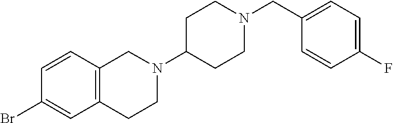 [SMILES: Fc1ccc(CN2CCC(N3CCc4cc(Br)ccc4C3)CC2)cc1] | +0.3 (100 μM) | 0 (100 μM) |
| 85 | 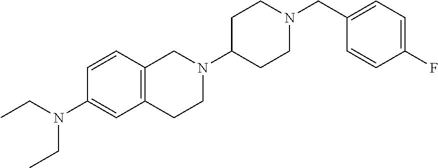 [SMILES: CCN(CC)c1ccc2c(c1)CCN(C1CCN(Cc3ccc(F)cc3)CC1)C2] | +14.0 (2 μM) | 0 (2 μM) |

### Example 3. Effects of Compound 2 on the Phosphorylation of FGFR1

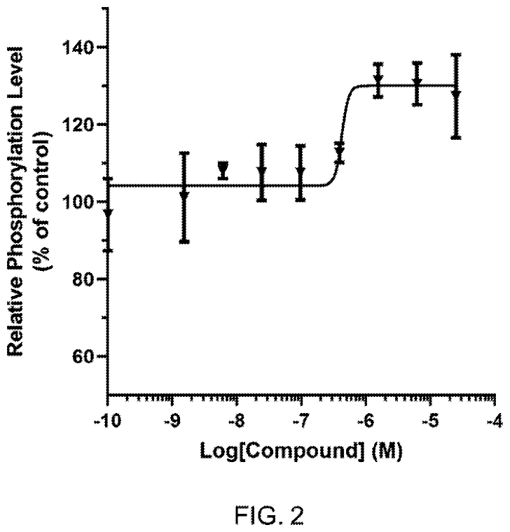

Cells expressing FGFR1 were exposed to increasing concentrations of Compound 2 in the presence of a submaximal concentration of FGF-2. Cells were then lysed, and the relative phosphorylation of FGFR1 was assessed using antibodies to non-phosphorylated and phosphorylated FGFR1. The results are shown in FIG. 2, which is a graph showing the phosphorylation of FGFR1 in the presence of increasing concentrations of Compound 2. The inflection point on the curve shows the concentration of Compound 2 at which it increases FGFR1 phosphorylation. The data indicates that Compound 2 augmented the effects of FGF-2.

### Example 4. Stroke Recovery In Vivo (Compound 2 Given on Day 1, 2, and 3 after Stroke)

Compound 2 (2-(1-benzhydrylpiperidin-4-yl)-N,N-diethyl-1,2,3,4-tetrahydroisoquinolin-6-amine) was tested for its effectiveness in a rodent model of stroke recovery. Twenty male Sprague Dawley Rats (Charles River Laboratories) each weighing 300-400 g were used in this experiment. First, anesthesia was induced in an induction chamber with 2-3% isoflurane in N₂O:O₂ (2:1) and maintained with 1-1.5% isoflurane via face mask. Adequate depth of anesthesia was assessed by lack of withdrawal to hindlimb pinch and loss of eyeblink reflex. Once anesthetized, animals received cefazolin sodium (40 mg/kg, i.p.) and buprenorphine SR (0.9-1 mg/kg, s.c.). Cefazolin was used as a prophylactic antibiotic. A veterinary ophthalmic ointment (Sodium Chloride hypertonicity ophthalmic ointment (Muro 128 Sterile Ophthalmic 5% Ointment)) was applied to the eyes.

A small focal stroke (infarct) was made on the right side of the surface of the brain (cerebral cortex) by middle cerebral artery occlusion (MCAO). The stroke becomes fixed in size and location within 24 hours after the MCAO. The stroke results in impaired sensorimotor function of the contralateral (left) limbs that recover slowly and incompletely over time.

For stroke surgery, the right side of the head was shaved with electric clippers (patch of approximately 3 cm by 5 cm between eye and ear). The region was carefully cleaned with Hibiclens and alcohol. Using aseptic technique, an incision was made midway between the eye and eardrum canal. The temporalis muscle was isolated, bisected, and reflected. A small window of bone was removed via drill and rongeurs (subtemporal craniectomy) to expose the MCA. Care was taken not to remove the zygomatic arch or to transect the facial nerve that would impair the ability of the animal to chew after surgery. Using a dissecting microscope, the dura was incised, and the MCA was electrocoagulated from just proximal to the olfactory tract to the inferior cerebral vein (taking care not to rupture this vein), using microbipolar electrocauterization. The MCA was then transected. The temporalis muscle was then repositioned, and the incision was closed subcutaneously with sutures. The skin incision was closed with surgical staples (2-3 required). Throughout the procedure, body temperature was maintained at 37.0°±1° C. using a self-regulating heating pad connected to a rectal thermometer. Following surgery, animals remained on a heating pad until they woke up from anesthesia. They were returned to clean home cages. The animals were housed 2 per cage before and after surgery, unless severe aggression was displayed, or death of cage mate(s). They were observed frequently on the day of MCAO surgery (Day 0) and at least once daily thereafter.

The rats were randomly assigned into two groups of ten each. Each group was injected intravenously (i.v.) with 2 ml/kg Compound 2 at 10 mg/kg or vehicle (18% Cremophor RH40 and 10% DMSO in 5% dextrose solution (D5WW)) on Day 1, 2, and 3 after MCAO. Day 0 is the day of the MCAO, and the days after the MCAO are numbered consecutively (Day 1, Day 2, Day 3, etc.) D-pre represents the day prior to the MCAO.

Behavioral evaluations of sensorimotor function were done by investigators blinded to treatment assignment. Limb placing tests were done on Day Pre (one day pre-MCAO operation), Day 1, Day 3, Day 4, Day 7, Day 14, and Day 21. The limb placing tests were divided into forelimb and hindlimb tests. For the forelimb-placing test, the examiner held the rat close to a tabletop and scored the rat's ability to place the forelimb on the tabletop in response to whisker, visual, tactile, or proprioceptive stimulation. Similarly, for the hindlimb placing test, the examiner assessed the rat's ability to place the hindlimb on the tabletop in response to tactile and proprioceptive stimulation. Separate sub-scores were obtained for each mode of sensory input and added to give total scores (for the forelimb placing test: 0=normal, 12=maximally impaired; for the hindlimb placing test: 0=normal; 6=maximally impaired). Scores were given in half-point increments (see below).
Forelimb placing test (0-12):
   whisker placing (0-2);
   visual placing (forward (0-2), sideways (0-2))
   tactile placing (dorsal (0-2), lateral (0-2))
   proprioceptive placing (0-2).
Hindlimb placing test (0-6):
   tactile placing (dorsal (0-2), lateral (0-2))
   proprioceptive placing (0-2).
   For each subtest, animals are scored as followed:
      0.0=immediate response
      0.5=response within 2 seconds
      1.0=response of 2-3 seconds
      1.5=response of >3 seconds
      2.0=no response

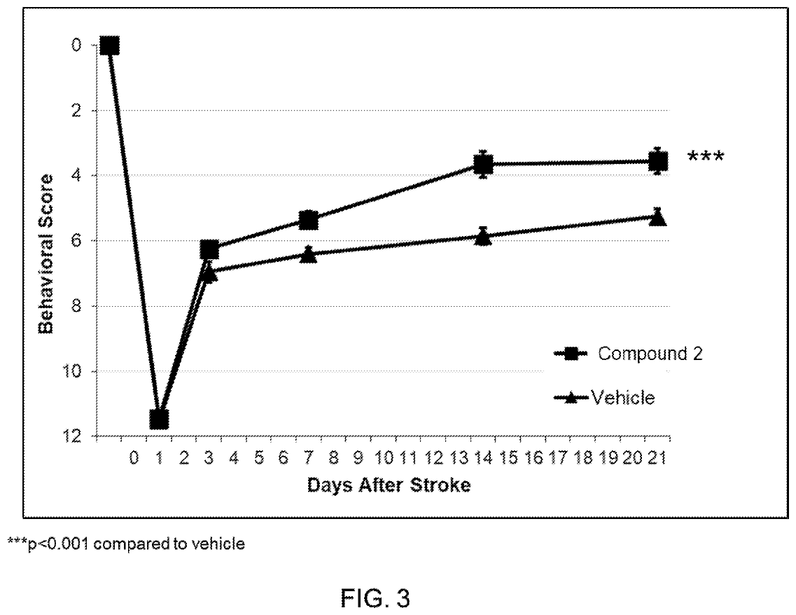

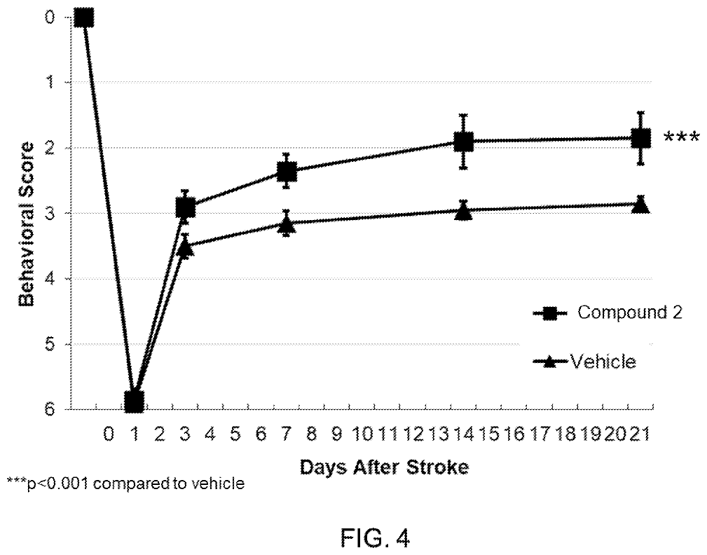

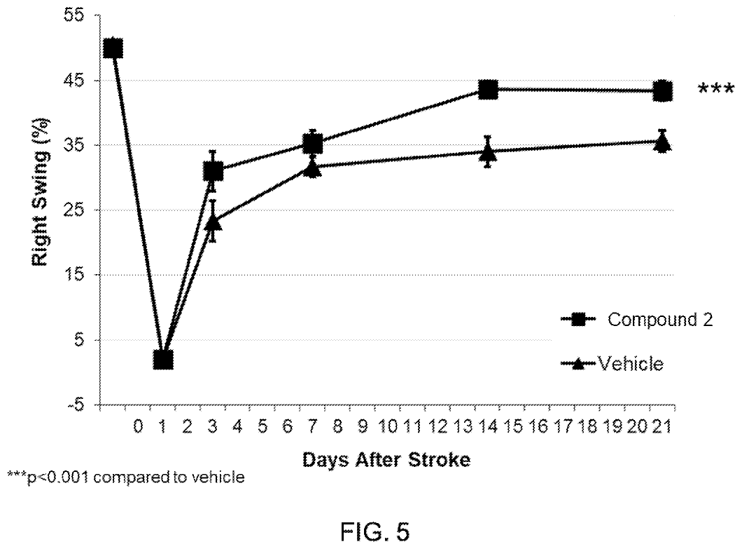

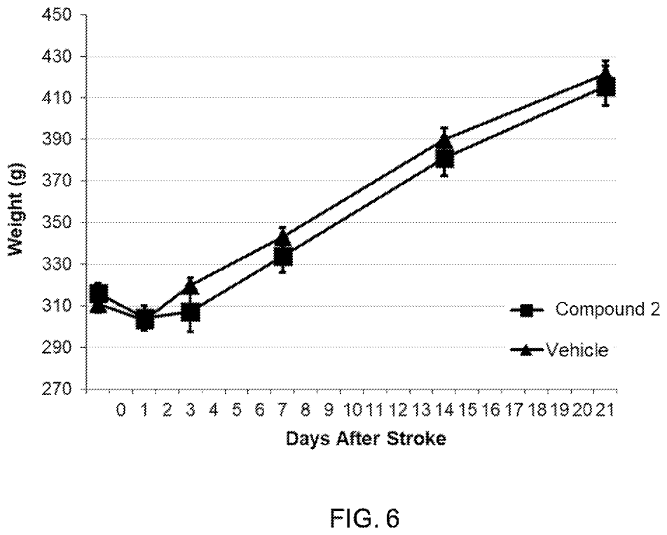

The results from limb placing tests, body swing tests, and body weight pre- and post-MCAO are shown in FIGS. 3-6.

Typically, after an initial rapid rise, there is a continued slow, steady, and partial improvement in sensorimotor function (as measured by forelimb and hindlimb placing and body swing tests) during the first three weeks after stroke. Previous studies using this model indicate that recovery plateaus at this time and does not change thereafter. Animals treated with Compound 2 showed a clear and significant augmentation of sensorimotor recovery on all three measures compared to vehicle-treated animals (p<0.001 by two-way repeated-measures ANOVA). The normal rise in body weight following surgery was not affected by treatment with Compound 2.

Treatment with Compound 2 was initiated at one day after stroke, at a time when infarct size and location is fixed. This indicates that Compound 2 does not promote enhanced recovery by reduction of infarct size, but rather through a separate recovery-promoting mechanism.

### Other Embodiments

Various modifications and variations of the described compositions, methods, and uses of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed is:

1. A compound of formula (I): 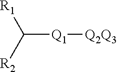
wherein
R₁ is H, optionally substituted C₃-C₂₀ cycloalkyl, optionally substituted C₄-C₂₀ cycloalkenyl, optionally substituted C₁-C₁₅ heterocyclyl, or optionally substituted C₆-C₁₆ aryl;
R₂ is optionally substituted C₃-C₂₀ cycloalkyl, optionally substituted C₄-C₂₀ cycloalkenyl, optionally substituted C₁-C₁₅ heterocyclyl, or optionally substituted C₆-C₁₆ aryl;
Q₁ is optionally substituted 4-to-6 membered heterocyclylene containing at least one nitrogen atom;
Q₂ is optionally substituted 5-to-7 membered heterocyclyl containing at least one nitrogen atom; and
Q₃ is optionally substituted C₁-C₁₅ heterocyclyl, optionally substituted C₆-C₁₆ aryl, optionally substituted C₃-C₂₀ cycloalkyl, or optionally substituted C₄-C₂₀ cycloalkenyl, wherein Q₃ is fused to Q₂,
or a pharmaceutically acceptable salt thereof.

2. The compound of claim 1, wherein Q₁ is 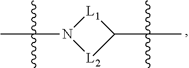
wherein each of L₁ and L₂ is, independently, optionally substituted C₁-C₂ alkylene.

3. The compound of claim 1 or 2, wherein Q₁ is

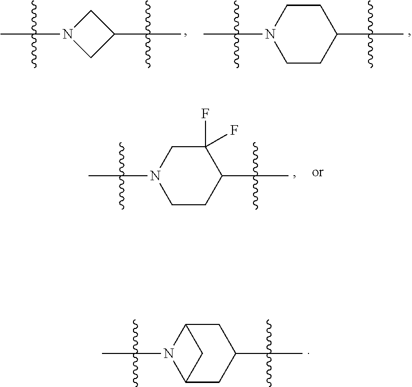 [SMILES: *C1CN(*)C1 ; *C1CCN(*)CC1 ; *C1CCN(*)CC1(F)F ; *C1CC2CC(C1)N2*]

4. The compound of any one of claims 1-3, wherein Q₂ is 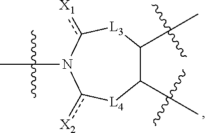
wherein each of L₃ and L₄ is independently absent or optionally substituted methylene; each [symbol] is independently a single or double bond; and each of X₁ and X₂ is independently O when [symbol] is a double bond or H when [symbol] is a single bond.

5. The compound of claim 4, wherein Q₂ is

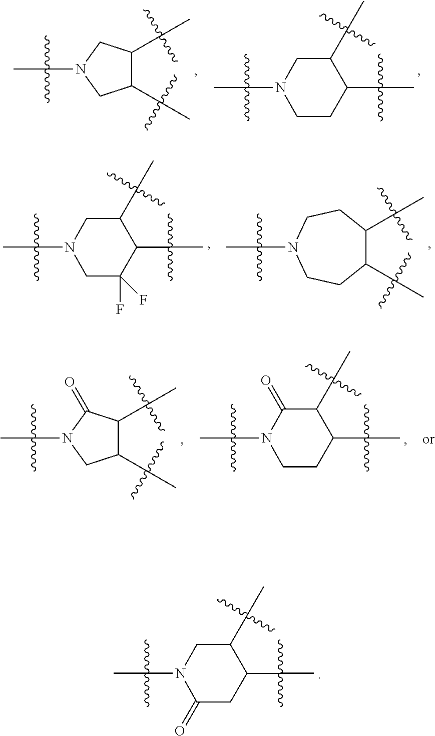

6. The compound of any one of claims 1-5, wherein Q₃ is optionally substituted phenyl or optionally substituted 6-membered aromatic heterocyclyl comprising at least one N atom.

7. The compound of claim 6, wherein Q₃ is 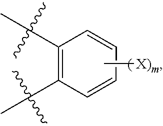
wherein m is 0-2, and each X is independently halo; CN; NO₂; optionally substituted C₁-C₆ alkyl; ORₐ, wherein Ra is H or optionally substituted C₁-C₆ alkyl; optionally substituted C₃-C₈ cycloalkyl; or NR_{b}R_{c}, wherein each of R_{b} and R_{c} is independently H or optionally substituted C₁-C₆ alkyl, or R_{b} and R_{c}, together with the N atom to which they are attached, form optionally substituted 3-7 membered heterocyclyl.

8. The compound of claim 7, wherein Q₃ is

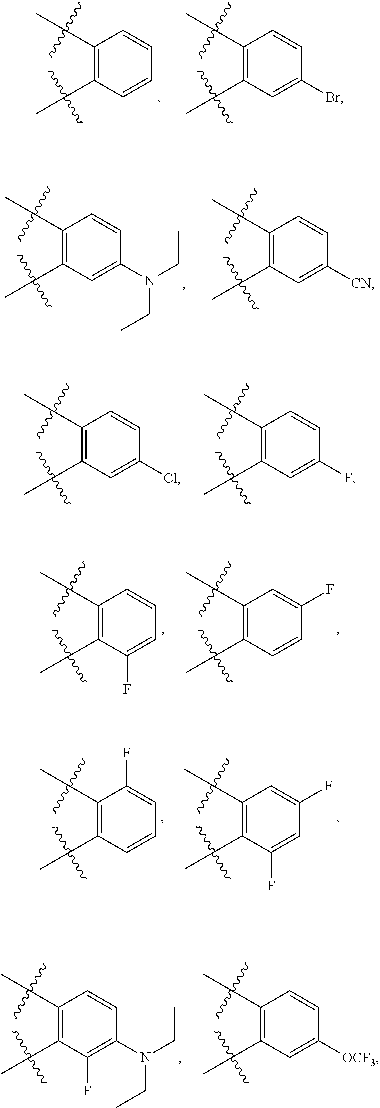

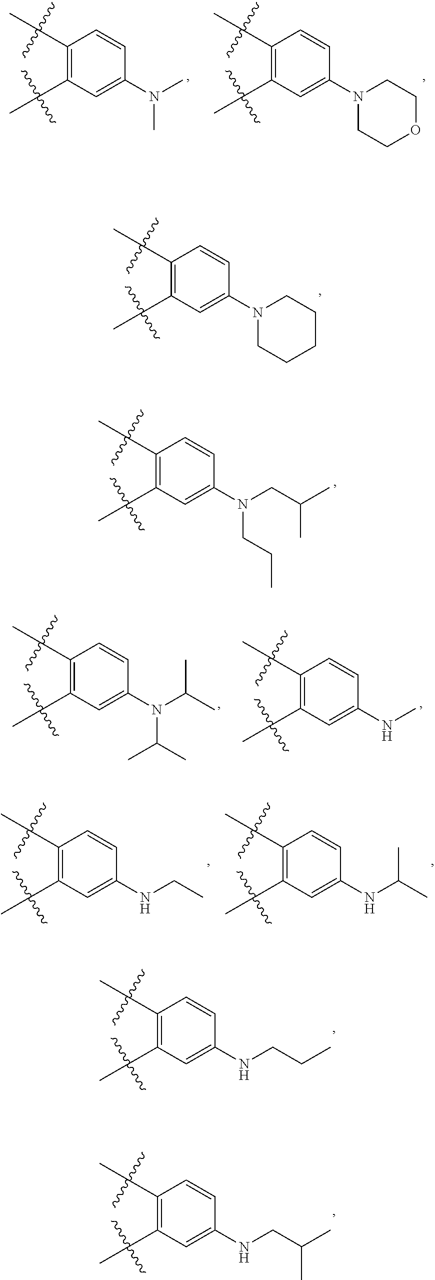

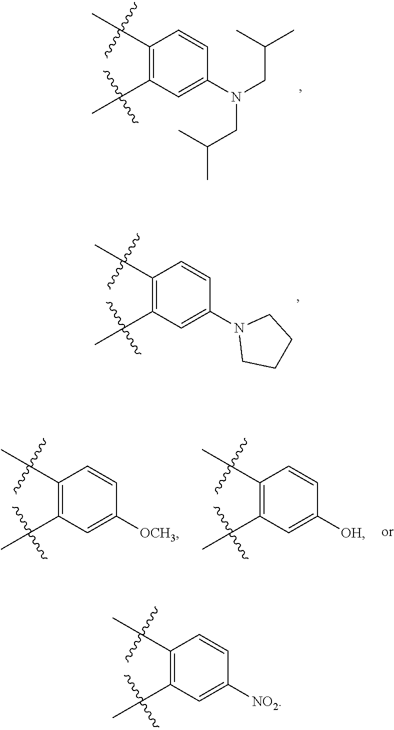

9. The compound of claim 6, wherein Q₃ is 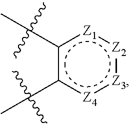
wherein each of Z₁, Z₂, Z₃, and Z₄ is N or CR_{c} and up to two of Z₁, Z₂, Z₃, and Z₄ is N, wherein each R_{c} is independently H; halo, CN; NO₂; optionally substituted C₁-C₆ alkyl; ORₐ, wherein Ra is H or optionally substituted C₁-C₆ alkyl; optionally substituted C₃-C₈ cycloalkyl; or NR_{b}R_{c}, wherein each of R_{b} and R_{c} is independently H or optionally substituted C₁-C₆ alkyl, or R_{b} and R_{c}, together with the N atom to which they are attached, form optionally substituted 3-7 membered heterocyclyl.

10. The compound of claim 9, wherein Q₃ is

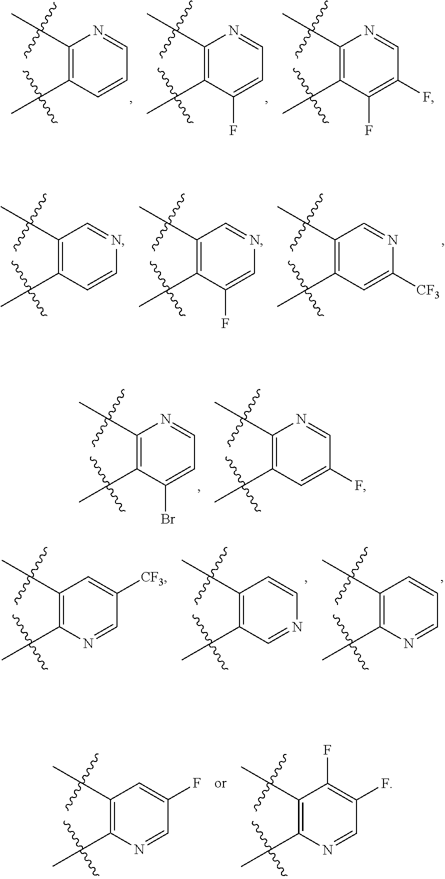

11. The compound of claim 1, wherein the compound is described by formula (II): 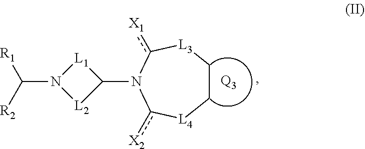
wherein
L₁ and L₂ are each independently —C(X₃)₂— or —(C(X₃)₂)₂—, wherein each X₃ is independently H, halo, CN, NO₂, or C₁-C₆ alkyl; or an X₃ in L₁ and an X₃ in L₂ combine to form C₁-C₃ alkylene;
each of L₃ and L₄ is independently absent or —C(X₄)₂—, wherein each X₄ is independently H, halo, CN, NO₂, or C₁-C₆ alkyl;
each [symbol] is independently a single or double bond; and
each of X₁ and X₂ is independently O when [symbol] is a double bond or H when [symbol] is a single bond.

12. The compound of claim 11, wherein the compound is described by formula (IIA): 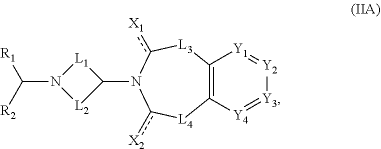
wherein each of Y₁, Y₂, Y₃, and Y₄ is independently N or CR₃, and at least one of Y₁, Y₂, Y₃, and Y₄ is CR₃, wherein each R₃ is independently H, halo, CN, NO₂, optionally substituted C₁-C₆ alkyl, optionally substituted C₂-C₆ alkenyl, optionally substituted C₂-C₆ alkynyl, optionally substituted C₃-C₈ cycloalkyl, optionally substituted C₄-C₈ cycloalkenyl, optionally substituted C₁-C₁₅ heterocyclyl, optionally substituted C₆-C₁₆ aryl, OR₄, SR₄, NR₄R₅, or C(O)NR₄R₅, wherein each of R₄ and R₅ is independently H, optionally substituted C₁-C₆ alkyl, optionally substituted C₂-C₆ alkenyl, optionally substituted C₂-C₆ alkynyl, optionally substituted C₃-C₈ cycloalkyl, optionally substituted C₄-C₈ cycloalkenyl, optionally substituted C₆-C₁₆ aryl, or optionally substituted C₁-C₁₅ heterocyclyl,
or a pharmaceutically acceptable salt.

13. The compound of claim 12, wherein each of Y₁, Y₂, Y₃, and Y₄ is CR₃.

14. The compound of claim 13, wherein each of Y₁, Y₂, and Y₃ is CH and Y₄ is CR₃.

15. The compound of claim 13, wherein each of Y₁, Y₂, and Y₄ is CH and Y₃ is CR₃.

16. The compound of claim 14 or 15, wherein R₃ is NR₄R₅.

17. The compound of claim 16, wherein each of R₄ and R₅ is independently H or optionally substituted C₁-C₆ alkyl.

18. The compound of claim 17, wherein R₃ is

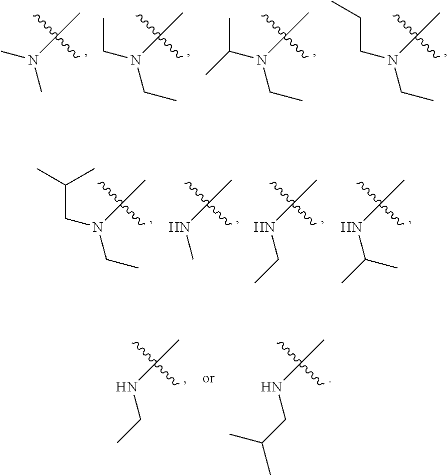

19. The compound of claim 14 or 15, wherein R₃ is optionally substituted C₁-C₁₅ heterocyclyl.

20. The compound of claim 19, wherein R₃ is

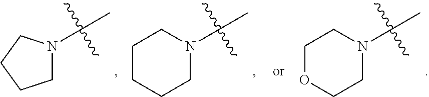 [SMILES: *N1CCCC1 ; *N1CCCCC1 ; *N1CCOCC1]

21. The compound of claim 14 or 15, wherein R₃ is halo.

22. The compound of claim 14 or 15, wherein R₃ is OCH₃, OCF₃, OH, CN, or N₀₂.

23. The compound of claim 12, wherein each of Y₁, Y₂, and Y₃ is CR₃ and Y₄ is N.

24. The compound of claim 12, wherein each of Y₁, Y₂, and Y₄ is CR₃ and Y₃ is N.

25. The compound of claim 23 or 24, wherein each R₃ is independently H or optionally substituted C₁-C₆ alkyl.

26. The compound of claim 25, wherein each R₃ is H.

27. The compound of claim 11, wherein Q₃ is optionally substituted C₁-C₄ heterocyclyl.

28. The compound of claim 27, wherein Q₃ is optionally substituted thiophene, optionally substituted pyrrole, optionally substituted furan, optionally substituted thiazole, optionally substituted oxazole, optionally substituted isothiazole, optionally substituted isooxazole, optionally substituted diazole, optionally substituted oxadiazole, optionally substituted thiadiazole, or optionally substituted triazole.

29. The compound of any one of claims 11-28, wherein each of L₁ and L₂ is —(CH₂)₂—.

30. The compound of any one of claims 11-28, wherein each of L₁ and L₂ is —CH₂—.

31. The compound of any one of claims 11-28, wherein L₁ is —CH₂CF₂— and L₂ is —(CH₂)₂—.

32. The compound of any one of claims 11-28, wherein an X₃ in L₁ and an X₃ in L₂ combine to form C₁-C₃ alkylene.

33. The compound of any one of claims 11-32, wherein X₁ is O and X₂ is H.

34. The compound of any one of claims 11-32, wherein X₁ is H and X₂ is O.

35. The compound of any one of claims 1-34, wherein each of R₁ and R₂ is optionally substituted C₆-C₁₆ aryl.

36. The compound of claim 35, wherein each of R₁ and R₂ is optionally substituted phenyl.

37. The compound of claim 36, wherein each of R₁ and R₂ is phenyl.

38. The compound of claim 36, wherein each of R₁ and R₂ is 4-fluorophenyl.

39. The compound of one of claims 1-34, wherein R₁ is H and R₂ is optionally substituted C₆-C₁₆ aryl.

40. The compound of claim 39, wherein R₂ is optionally substituted phenyl.

41. The compound of claim 40, wherein R₂ is phenyl.

42. The compound of claim 40, wherein R₂ is 4-fluorophenyl.

43. The compound of claim 40, wherein R₂ is 3-fluorophenyl.

44. The compound of claim 1, wherein the compound is a compound of Table 1 or a pharmaceutically acceptable salt thereof:
TABLE 1

| Compound | Structure |
|---|---|
| 1 | 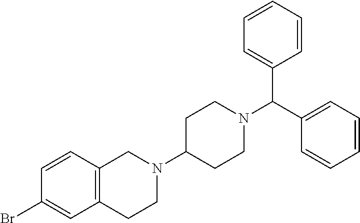 [SMILES: Brc1ccc2c(c1)CCN(C1CCN(C(c3ccccc3)c3ccccc3)CC1)C2] |
| 2 | 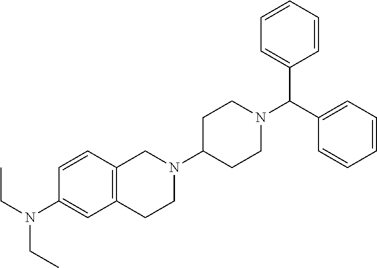 [SMILES: CCN(CC)c1ccc2c(c1)CCN(C1CCN(C(c3ccccc3)c3ccccc3)CC1)C2] |
| 3 | 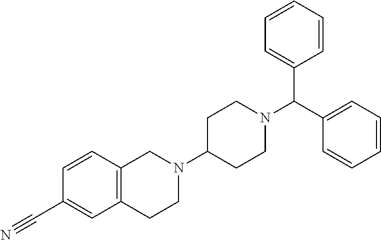 [SMILES: N#Cc1ccc2c(c1)CCN(C1CCN(C(c3ccccc3)c3ccccc3)CC1)C2] |
| 4 | 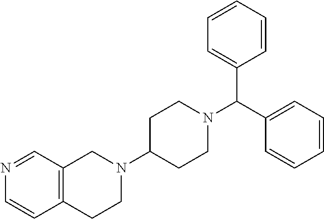 [SMILES: c1ccc(C(c2ccccc2)N2CCC(N3CCc4ccncc4C3)CC2)cc1] |
| 5 | 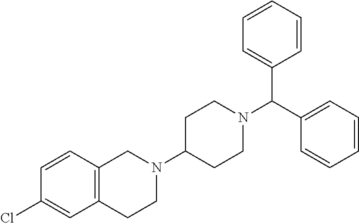 [SMILES: Clc1ccc2c(c1)CCN(C1CCN(C(c3ccccc3)c3ccccc3)CC1)C2] |
| 6 | 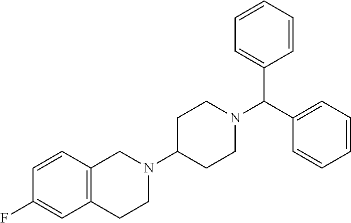 [SMILES: Fc1ccc2c(c1)CCN(C1CCN(C(c3ccccc3)c3ccccc3)CC1)C2] |
| 7 | 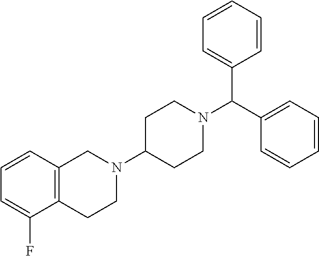 [SMILES: Fc1cccc2c1CCN(C1CCN(C(c3ccccc3)c3ccccc3)CC1)C2] |
| 8 | 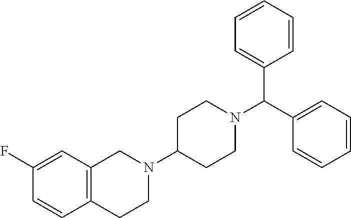 [SMILES: Fc1ccc2c(c1)CN(C1CCN(C(c3ccccc3)c3ccccc3)CC1)CC2] |
| 9 | 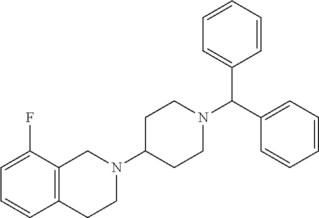 [SMILES: Fc1cccc2c1CN(C1CCN(C(c3ccccc3)c3ccccc3)CC1)CC2] |
| 10 | 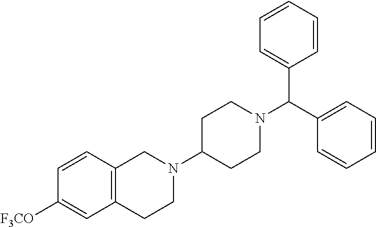 [SMILES: FC(F)(F)Oc1ccc2c(c1)CCN(C1CCN(C(c3ccccc3)c3ccccc3)CC1)C2] |
| 11 | 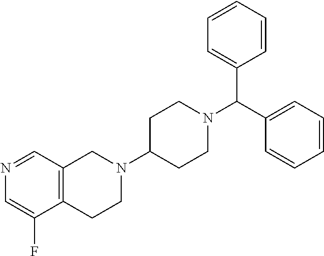 [SMILES: Fc1cncc2c1CCN(C1CCN(C(c3ccccc3)c3ccccc3)CC1)C2] |
| 12 | 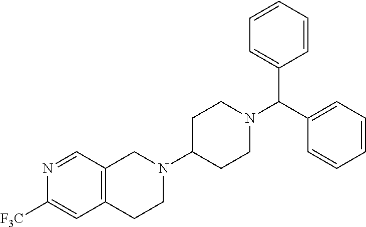 [SMILES: FC(F)(F)c1cc2c(cn1)CN(C1CCN(C(c3ccccc3)c3ccccc3)CC1)CC2] |
| 13 | 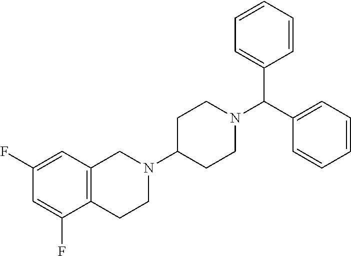 [SMILES: Fc1cc(F)c2c(c1)CN(C1CCN(C(c3ccccc3)c3ccccc3)CC1)CC2] |
| 14 | 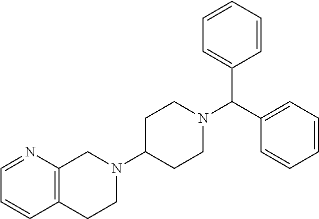 [SMILES: c1ccc(C(c2ccccc2)N2CCC(N3CCc4cccnc4C3)CC2)cc1] |
| 15 | 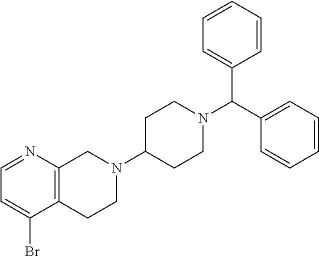 [SMILES: Brc1ccnc2c1CCN(C1CCN(C(c3ccccc3)c3ccccc3)CC1)C2] |
| 16 | 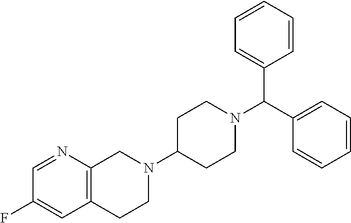 [SMILES: Fc1cnc2c(c1)CCN(C1CCN(C(c3ccccc3)c3ccccc3)CC1)C2] |
| 17 | 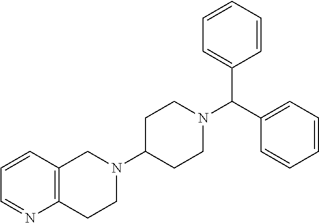 [SMILES: c1ccc(C(c2ccccc2)N2CCC(N3CCc4ncccc4C3)CC2)cc1] |
| 18 | 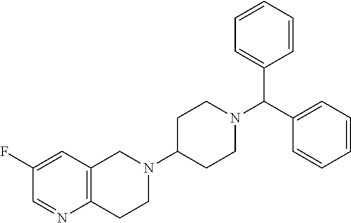 [SMILES: Fc1cnc2c(c1)CN(C1CCN(C(c3ccccc3)c3ccccc3)CC1)CC2] |
| 19 | 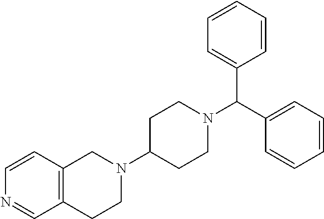 [SMILES: c1ccc(C(c2ccccc2)N2CCC(N3CCc4cnccc4C3)CC2)cc1] |
| 20 | 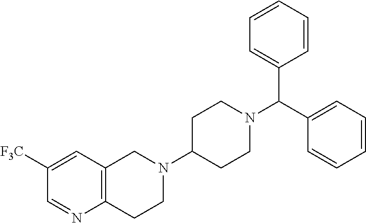 [SMILES: FC(F)(F)c1cnc2c(c1)CN(C1CCN(C(c3ccccc3)c3ccccc3)CC1)CC2] |
| 21 | 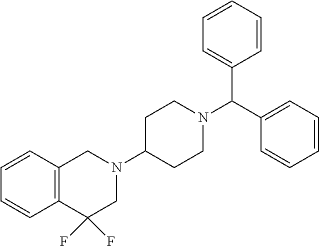 [SMILES: FC1(F)CN(C2CCN(C(c3ccccc3)c3ccccc3)CC2)Cc2ccccc21] |
| 22 | 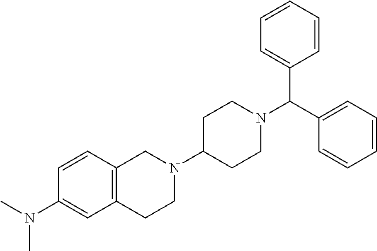 [SMILES: CN(C)c1ccc2c(c1)CCN(C1CCN(C(c3ccccc3)c3ccccc3)CC1)C2] |
| 23 | 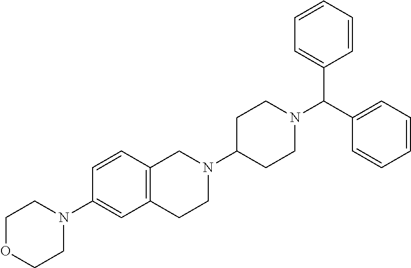 [SMILES: c1ccc(C(c2ccccc2)N2CCC(N3CCc4cc(N5CCOCC5)ccc4C3)CC2)cc1] |
| 24 | 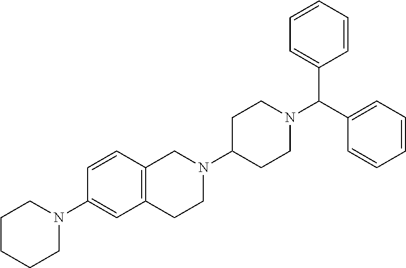 [SMILES: c1ccc(C(c2ccccc2)N2CCC(N3CCc4cc(N5CCCCC5)ccc4C3)CC2)cc1] |
| 25 | 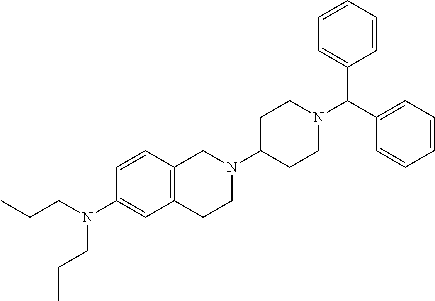 [SMILES: CCCN(CCC)c1ccc2c(c1)CCN(C1CCN(C(c3ccccc3)c3ccccc3)CC1)C2] |
| 26 | 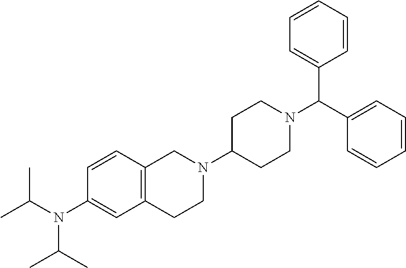 [SMILES: CC(C)N(c1ccc2c(c1)CCN(C1CCN(C(c3ccccc3)c3ccccc3)CC1)C2)C(C)C] |
| 27 | 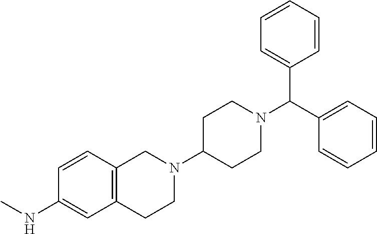 [SMILES: CNc1ccc2c(c1)CCN(C1CCN(C(c3ccccc3)c3ccccc3)CC1)C2] |
| 28 | 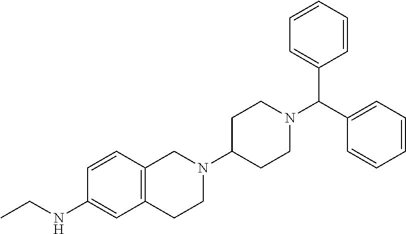 [SMILES: CCNc1ccc2c(c1)CCN(C1CCN(C(c3ccccc3)c3ccccc3)CC1)C2] |
| 29 | 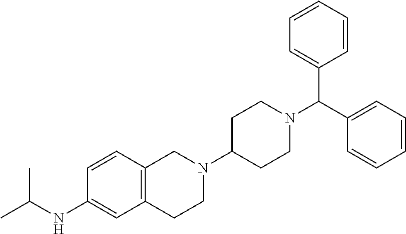 [SMILES: CC(C)Nc1ccc2c(c1)CCN(C1CCN(C(c3ccccc3)c3ccccc3)CC1)C2] |
| 30 | 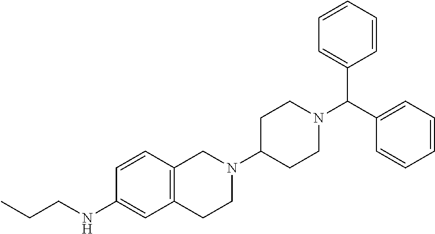 [SMILES: CCCNc1ccc2c(c1)CCN(C1CCN(C(c3ccccc3)c3ccccc3)CC1)C2] |
| 31 | 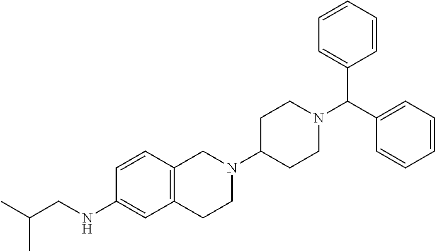 [SMILES: CC(C)CNc1ccc2c(c1)CCN(C1CCN(C(c3ccccc3)c3ccccc3)CC1)C2] |
| 32 | 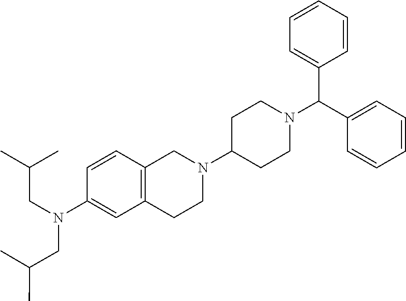 [SMILES: CC(C)CN(CC(C)C)c1ccc2c(c1)CCN(C1CCN(C(c3ccccc3)c3ccccc3)CC1)C2] |
| 33 | 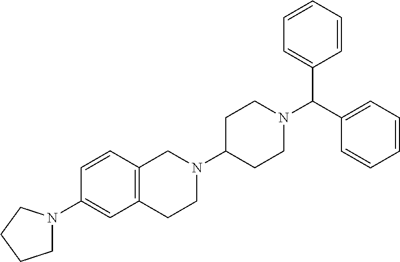 [SMILES: c1ccc(C(c2ccccc2)N2CCC(N3CCc4cc(N5CCCC5)ccc4C3)CC2)cc1] |
| 34 | 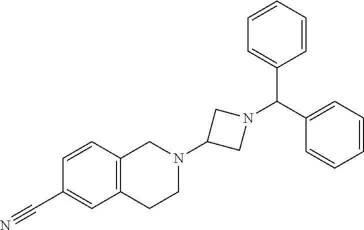 [SMILES: N#Cc1ccc2c(c1)CCN(C1CN(C(c3ccccc3)c3ccccc3)C1)C2] |
| 35 | 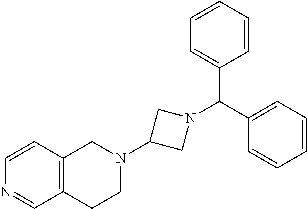 [SMILES: c1ccc(C(c2ccccc2)N2CC(N3CCc4cnccc4C3)C2)cc1] |
| 36 | 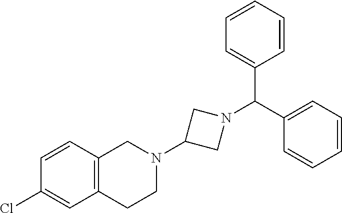 [SMILES: Clc1ccc2c(c1)CCN(C1CN(C(c3ccccc3)c3ccccc3)C1)C2] |
| 37 | 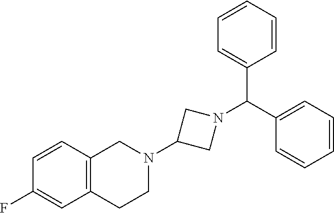 [SMILES: Fc1ccc2c(c1)CCN(C1CN(C(c3ccccc3)c3ccccc3)C1)C2] |
| 38 | 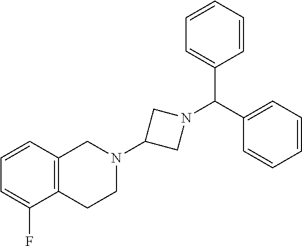 [SMILES: Fc1cccc2c1CCN(C1CN(C(c3ccccc3)c3ccccc3)C1)C2] |
| 39 | 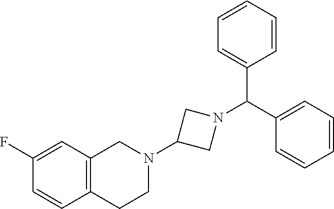 [SMILES: Fc1ccc2c(c1)CN(C1CN(C(c3ccccc3)c3ccccc3)C1)CC2] |
| 40 | 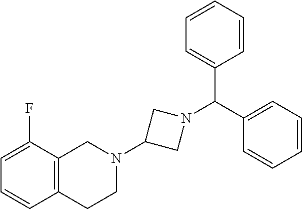 [SMILES: Fc1cccc2c1CN(C1CN(C(c3ccccc3)c3ccccc3)C1)CC2] |
| 41 | 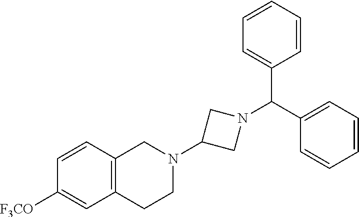 [SMILES: FC(F)(F)Oc1ccc2c(c1)CCN(C1CN(C(c3ccccc3)c3ccccc3)C1)C2] |
| 42 | 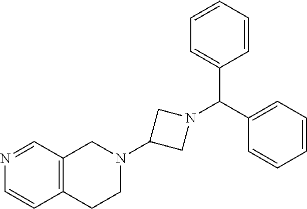 [SMILES: c1ccc(C(c2ccccc2)N2CC(N3CCc4ccncc4C3)C2)cc1] |
| 43 | 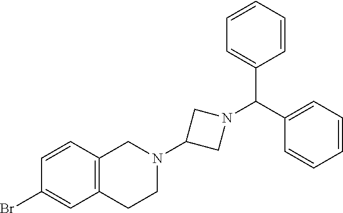 [SMILES: Brc1ccc2c(c1)CCN(C1CN(C(c3ccccc3)c3ccccc3)C1)C2] |
| 44 | 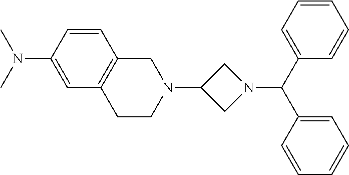 [SMILES: CN(C)c1ccc2c(c1)CCN(C1CN(C(c3ccccc3)c3ccccc3)C1)C2] |
| 45 | 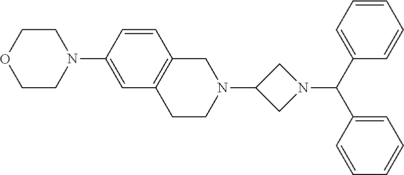 [SMILES: c1ccc(C(c2ccccc2)N2CC(N3CCc4cc(N5CCOCC5)ccc4C3)C2)cc1] |
| 46 | 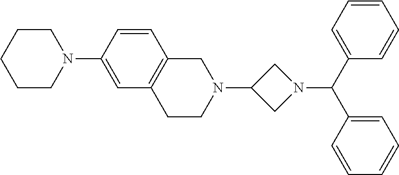 [SMILES: c1ccc(C(c2ccccc2)N2CC(N3CCc4cc(N5CCCCC5)ccc4C3)C2)cc1] |
| 47 | 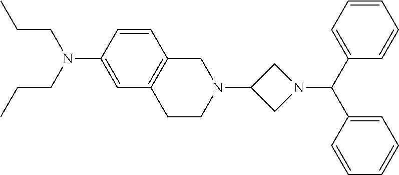 [SMILES: CCCN(CCC)c1ccc2c(c1)CCN(C1CN(C(c3ccccc3)c3ccccc3)C1)C2] |
| 48 | 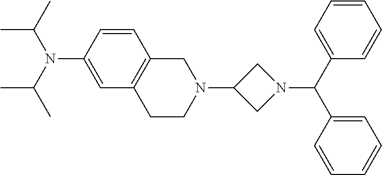 [SMILES: CC(C)N(c1ccc2c(c1)CCN(C1CN(C(c3ccccc3)c3ccccc3)C1)C2)C(C)C] |
| 49 | 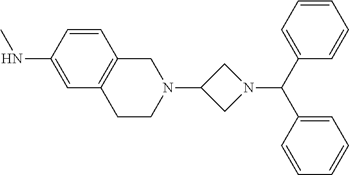 [SMILES: CNc1ccc2c(c1)CCN(C1CN(C(c3ccccc3)c3ccccc3)C1)C2] |
| 50 | 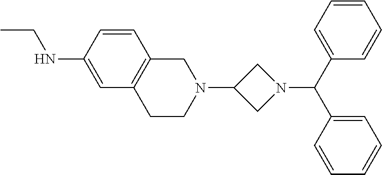 [SMILES: CCNc1ccc2c(c1)CCN(C1CN(C(c3ccccc3)c3ccccc3)C1)C2] |
| 51 | 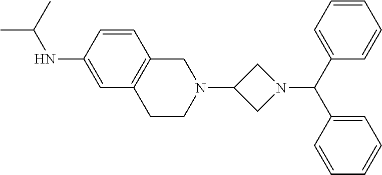 [SMILES: CC(C)Nc1ccc2c(c1)CCN(C1CN(C(c3ccccc3)c3ccccc3)C1)C2] |
| 52 | 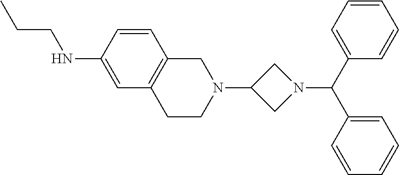 [SMILES: CCCNc1ccc2c(c1)CCN(C1CN(C(c3ccccc3)c3ccccc3)C1)C2] |
| 53 | 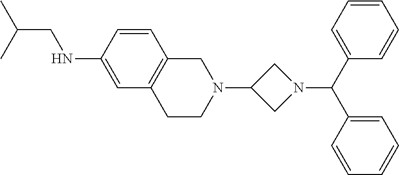 [SMILES: CC(C)CNc1ccc2c(c1)CCN(C1CN(C(c3ccccc3)c3ccccc3)C1)C2] |
| 54 | 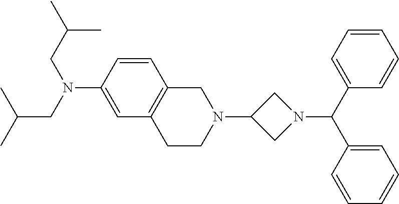 [SMILES: CC(C)CN(CC(C)C)c1ccc2c(c1)CCN(C1CN(C(c3ccccc3)c3ccccc3)C1)C2] |
| 55 | 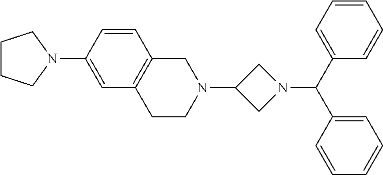 [SMILES: c1ccc(C(c2ccccc2)N2CC(N3CCc4cc(N5CCCC5)ccc4C3)C2)cc1] |
| 56 | 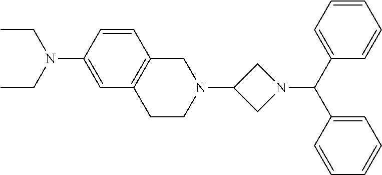 [SMILES: CCN(CC)c1ccc2c(c1)CCN(C1CN(C(c3ccccc3)c3ccccc3)C1)C2] |
| 57 | 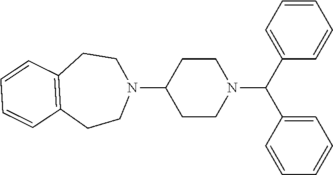 [SMILES: c1ccc(C(c2ccccc2)N2CCC(N3CCc4ccccc4CC3)CC2)cc1] |
| 58 | 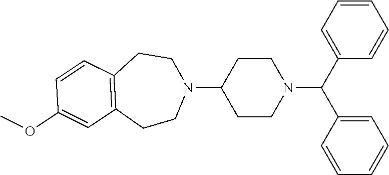 [SMILES: COc1ccc2c(c1)CCN(C1CCN(C(c3ccccc3)c3ccccc3)CC1)CC2] |
| 59 | 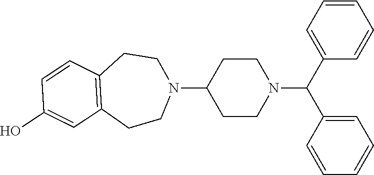 [SMILES: Oc1ccc2c(c1)CCN(C1CCN(C(c3ccccc3)c3ccccc3)CC1)CC2] |
| 60 | 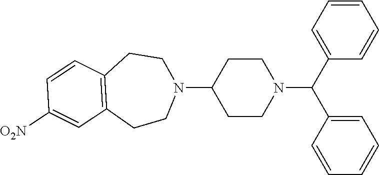 [SMILES: O=[N+]([O-])c1ccc2c(c1)CCN(C1CCN(C(c3ccccc3)c3ccccc3)CC1)CC2] |
| 61 | 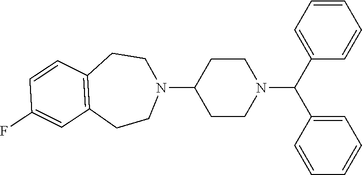 [SMILES: Fc1ccc2c(c1)CCN(C1CCN(C(c3ccccc3)c3ccccc3)CC1)CC2] |
| 62 | 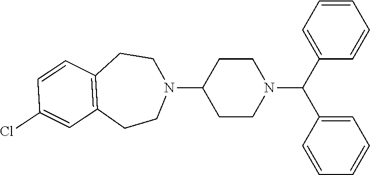 [SMILES: Clc1ccc2c(c1)CCN(C1CCN(C(c3ccccc3)c3ccccc3)CC1)CC2] |
| 63 | 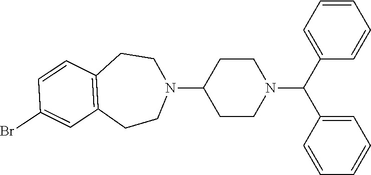 [SMILES: Brc1ccc2c(c1)CCN(C1CCN(C(c3ccccc3)c3ccccc3)CC1)CC2] |
| 64 | 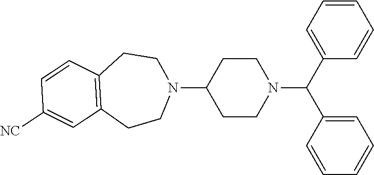 [SMILES: N#Cc1ccc2c(c1)CCN(C1CCN(C(c3ccccc3)c3ccccc3)CC1)CC2] |
| 65 | 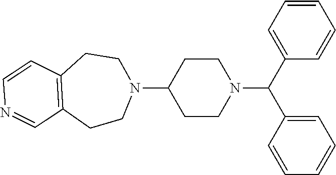 [SMILES: c1ccc(C(c2ccccc2)N2CCC(N3CCc4ccncc4CC3)CC2)cc1] |
| 66 | 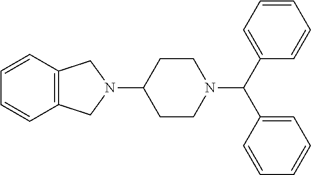 [SMILES: c1ccc(C(c2ccccc2)N2CCC(N3Cc4ccccc4C3)CC2)cc1] |
| 67 | 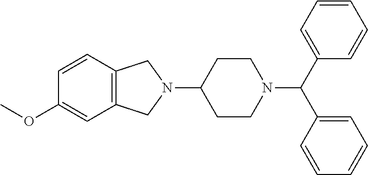 [SMILES: COc1ccc2c(c1)CN(C1CCN(C(c3ccccc3)c3ccccc3)CC1)C2] |
| 68 | 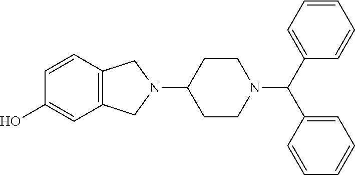 [SMILES: Oc1ccc2c(c1)CN(C1CCN(C(c3ccccc3)c3ccccc3)CC1)C2] |
| 69 | 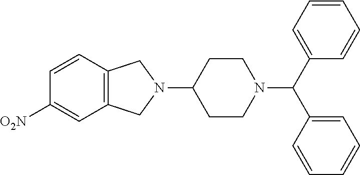 [SMILES: O=[N+]([O-])c1ccc2c(c1)CN(C1CCN(C(c3ccccc3)c3ccccc3)CC1)C2] |
| 70 | 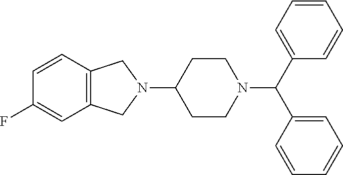 [SMILES: Fc1ccc2c(c1)CN(C1CCN(C(c3ccccc3)c3ccccc3)CC1)C2] |
| 71 | 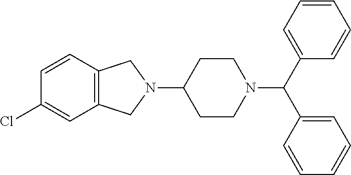 [SMILES: Clc1ccc2c(c1)CN(C1CCN(C(c3ccccc3)c3ccccc3)CC1)C2] |
| 72 | 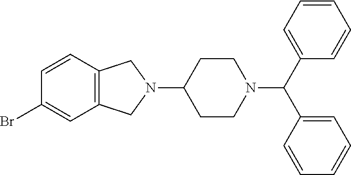 [SMILES: Brc1ccc2c(c1)CN(C1CCN(C(c3ccccc3)c3ccccc3)CC1)C2] |
| 73 | 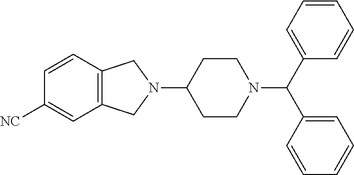 [SMILES: N#Cc1ccc2c(c1)CN(C1CCN(C(c3ccccc3)c3ccccc3)CC1)C2] |
| 74 | 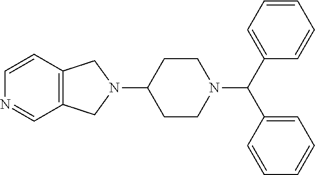 [SMILES: c1ccc(C(c2ccccc2)N2CCC(N3Cc4ccncc4C3)CC2)cc1] |
| 75 | 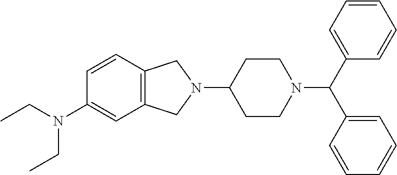 [SMILES: CCN(CC)c1ccc2c(c1)CN(C1CCN(C(c3ccccc3)c3ccccc3)CC1)C2] |
| 76 | 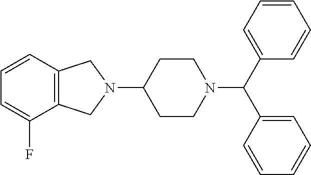 [SMILES: Fc1cccc2c1CN(C1CCN(C(c3ccccc3)c3ccccc3)CC1)C2] |
| 77 | 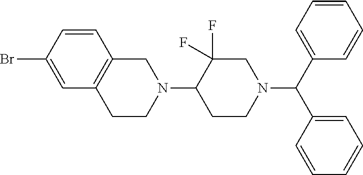 [SMILES: FC1(F)CN(C(c2ccccc2)c2ccccc2)CCC1N1CCc2cc(Br)ccc2C1] |
| 78 | 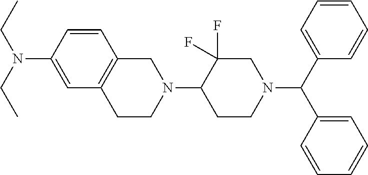 [SMILES: CCN(CC)c1ccc2c(c1)CCN(C1CCN(C(c3ccccc3)c3ccccc3)CC1(F)F)C2] |
| 79 | 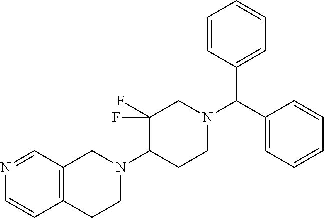 [SMILES: FC1(F)CN(C(c2ccccc2)c2ccccc2)CCC1N1CCc2ccncc2C1] |
| 80 | 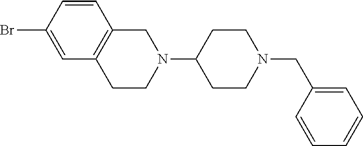 [SMILES: Brc1ccc2c(c1)CCN(C1CCN(Cc3ccccc3)CC1)C2] |
| 81 | 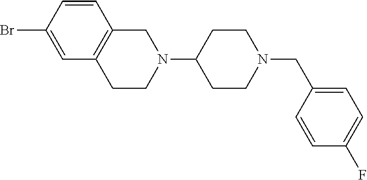 [SMILES: Fc1ccc(CN2CCC(N3CCc4cc(Br)ccc4C3)CC2)cc1] |
| 82 | 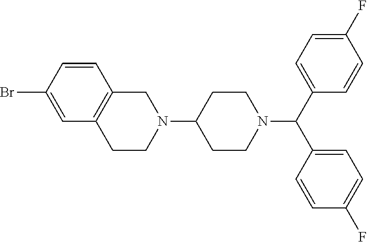 [SMILES: Fc1ccc(C(c2ccc(F)cc2)N2CCC(N3CCc4cc(Br)ccc4C3)CC2)cc1] |
| 83 | 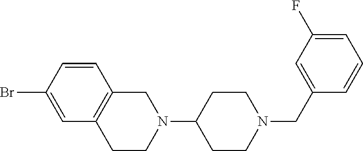 [SMILES: Fc1cccc(CN2CCC(N3CCc4cc(Br)ccc4C3)CC2)c1] |
| 84 | 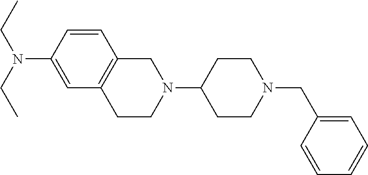 [SMILES: CCN(CC)c1ccc2c(c1)CCN(C1CCN(Cc3ccccc3)CC1)C2] |
| 85 | 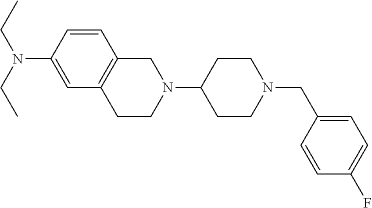 [SMILES: CCN(CC)c1ccc2c(c1)CCN(C1CCN(Cc3ccc(F)cc3)CC1)C2] |
| 86 | 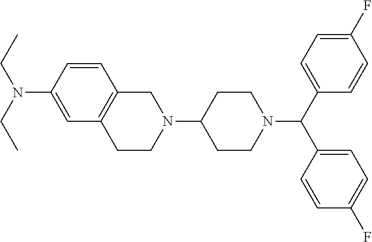 [SMILES: CCN(CC)c1ccc2c(c1)CCN(C1CCN(C(c3ccc(F)cc3)c3ccc(F)cc3)CC1)C2] |
| 87 | 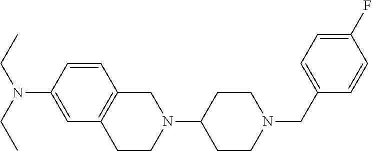 [SMILES: CCN(CC)c1ccc2c(c1)CCN(C1CCN(Cc3ccc(F)cc3)CC1)C2] |
| 88 | 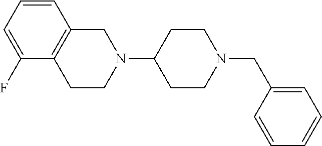 [SMILES: Fc1cccc2c1CCN(C1CCN(Cc3ccccc3)CC1)C2] |
| 89 | 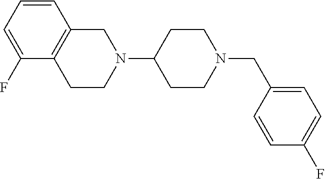 [SMILES: Fc1ccc(CN2CCC(N3CCc4c(F)cccc4C3)CC2)cc1] |
| 90 | 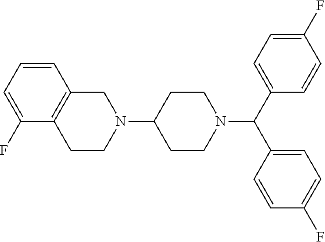 [SMILES: Fc1ccc(C(c2ccc(F)cc2)N2CCC(N3CCc4c(F)cccc4C3)CC2)cc1] |
| 91 | 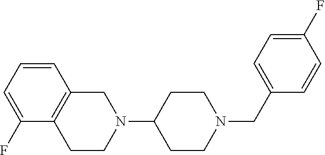 [SMILES: Fc1ccc(CN2CCC(N3CCc4c(F)cccc4C3)CC2)cc1] |
| 92 | 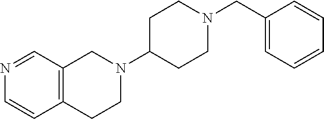 [SMILES: c1ccc(CN2CCC(N3CCc4ccncc4C3)CC2)cc1] |
| 93 | 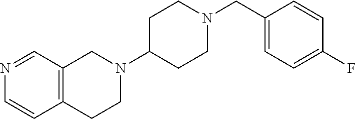 [SMILES: Fc1ccc(CN2CCC(N3CCc4ccncc4C3)CC2)cc1] |
| 94 | 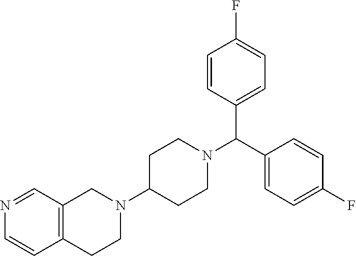 [SMILES: Fc1ccc(C(c2ccc(F)cc2)N2CCC(N3CCc4ccncc4C3)CC2)cc1] |
| 95 | 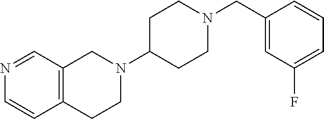 [SMILES: Fc1cccc(CN2CCC(N3CCc4ccncc4C3)CC2)c1] |
| 96 | 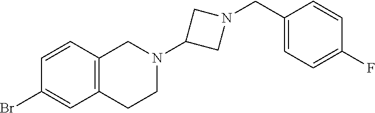 [SMILES: Fc1ccc(CN2CC(N3CCc4cc(Br)ccc4C3)C2)cc1] |
| 97 | 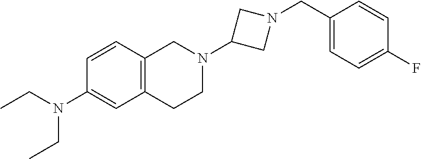 [SMILES: CCN(CC)c1ccc2c(c1)CCN(C1CN(Cc3ccc(F)cc3)C1)C2] |
| 98 | 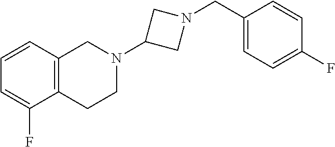 [SMILES: Fc1ccc(CN2CC(N3CCc4c(F)cccc4C3)C2)cc1] |
| 99 | 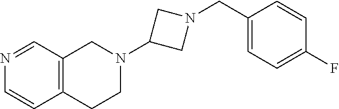 [SMILES: Fc1ccc(CN2CC(N3CCc4ccncc4C3)C2)cc1] |
| 100 | 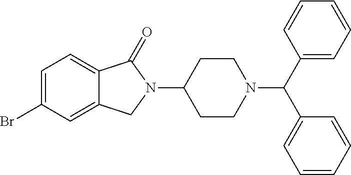 [SMILES: O=C1c2ccc(Br)cc2CN1C1CCN(C(c2ccccc2)c2ccccc2)CC1] |
| 101 | 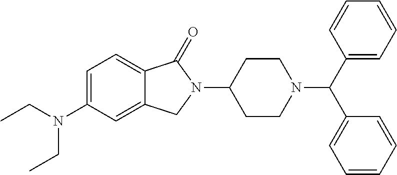 [SMILES: CCN(CC)c1ccc2c(c1)CN(C1CCN(C(c3ccccc3)c3ccccc3)CC1)C2=O] |
| 102 | 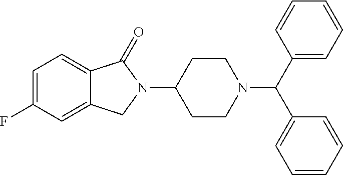 [SMILES: O=C1c2ccc(F)cc2CN1C1CCN(C(c2ccccc2)c2ccccc2)CC1] |
| 103 | 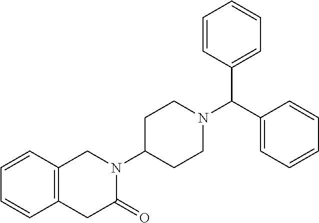 [SMILES: O=C1Cc2ccccc2CN1C1CCN(C(c2ccccc2)c2ccccc2)CC1] |
| 104 | 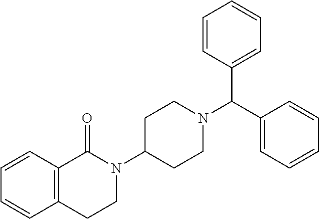 [SMILES: O=C1c2ccccc2CCN1C1CCN(C(c2ccccc2)c2ccccc2)CC1] |
| 105 | 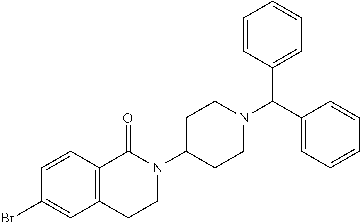 [SMILES: O=C1c2ccc(Br)cc2CCN1C1CCN(C(c2ccccc2)c2ccccc2)CC1] |
| 106 | 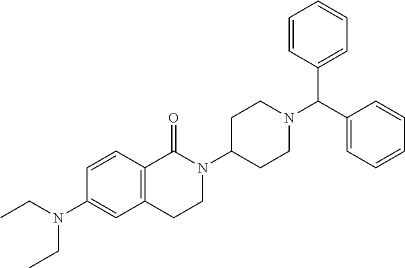 [SMILES: CCN(CC)c1ccc2c(c1)CCN(C1CCN(C(c3ccccc3)c3ccccc3)CC1)C2=O] |

45. The compound of claim 44, wherein the compound is Compound 2: 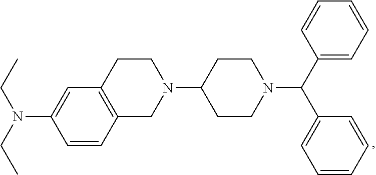 [SMILES: CCN(CC)c1ccc2c(c1)CCN(C1CCN(C(c3ccccc3)c3ccccc3)CC1)C2]
or a pharmaceutically acceptable salt thereof.

46. The compound of claim 44, wherein the compound is Compound 4: 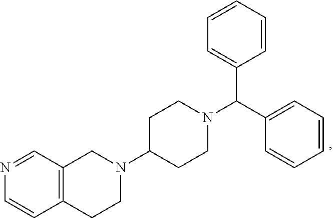 [SMILES: c1ccc(C(c2ccccc2)N2CCC(N3CCc4ccncc4C3)CC2)cc1]
or a pharmaceutically acceptable salt thereof.

47. The compound of claim 44, wherein the compound is Compound 7: 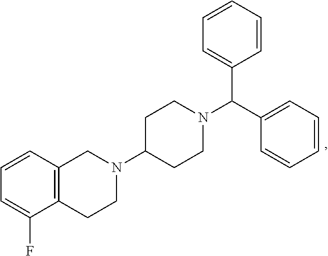 [SMILES: Fc1cccc2c1CCN(C1CCN(C(c3ccccc3)c3ccccc3)CC1)C2]
or a pharmaceutically acceptable salt thereof.

48. The compound of claim 44, wherein the compound is Compound 43: 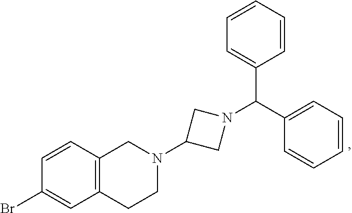 [SMILES: Brc1ccc2c(c1)CCN(C1CN(C(c3ccccc3)c3ccccc3)C1)C2]
or a pharmaceutically acceptable salt thereof.

49. The compound of claim 44, wherein the compound is Compound 85: 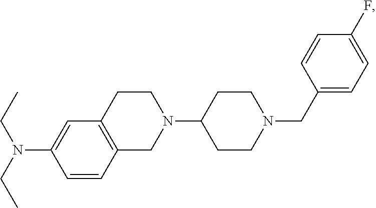 [SMILES: CCN(CC)c1ccc2c(c1)CCN(C1CCN(Cc3ccc(F)cc3)CC1)C2]
or a pharmaceutically acceptable salt thereof.

50. A pharmaceutical composition comprising the compound of any one of claims 1-49 or a pharmaceutically acceptable salt thereof and a pharmaceutically acceptable excipient.

51. A method of treating a subject having a disease or injury comprising administering to said subject a therapeutically effective amount of the pharmaceutical composition of claim 50.

52. The method of claim 51, wherein the disease or injury is stroke; congenital hypogonadotropic hypogonadism; cerebral hemorrhage; traumatic brain injury (TBI); spinal cord injury (SCI); peripheral vascular disease (PVD); wounds; bone or cartilage injury; hearing loss; depression; anxiety; post-traumatic stress disorder (PTSD); substance abuse; peripheral nerve injury; hematopoietic disorders; amyotrophic lateral sclerosis (ALS); Alzheimer's disease; Parkinson's disease; heart disease; non-arteritic ischemic optic neuropathy (NAION); retinal artery occlusion; bronchopulmonary dysplasia, muscular dystrophy, anosmia, aging, memory disturbance, or viral infection.

53. The method of claim 52, wherein the disease or injury is stroke.

54. The method of claim 53, wherein the stroke is acute stroke.

55. The method of claim 53, wherein the stroke is in a recovery phase.

56. The method of claim 52, wherein the disease or injury is congenital hypogonadotropic hypogonadism.

57. The method of claim 56, wherein the congenital hypogonadotropic hypogonadism is Kallmann Syndrome.

58. The method of claim 52, wherein the disease or injury is viral infection.

59. A method of increasing spermatogenesis in a subject comprising administering to said subject an effective amount of the pharmaceutical composition of claim 50.
